(19) United States
(12) Patent Application Publication
(10) Pub. No.: US 2024/0010606 A1
(43) Pub. Date: Jan. 11, 2024

(54) FUMARATE DERIVATIVES AND THEIR MEDICAL USE

(71) Applicant: Sitryx Therapeutics Limited, Oxford (GB)

(72) Inventors: Michael Liam COOKE, Nottingham, Nottinghamshire (GB); Matthew Colin Thor FYFE, Oxford (GB); Barry John TEOBALD, Oxford (GB)

(73) Assignee: Sitryx Therapeutics Limited, Oxford (GB)

(21) Appl. No.: 18/042,137

(22) PCT Filed: Aug. 20, 2021
(86) PCT No.: PCT/GB2021/052158

§ 371 Date: Feb. 17, 2023

(30) Foreign Application Priority Data
Aug. 21, 2020 (EP) 20192222.6
Mar. 1, 2021 (EP) 21159913.9
Jul. 1, 2021 (EP) 21183049.2

(51) Int. Cl. C07C 69/003; C07D 257/02

(52) U.S. Cl. CPC C07C 69/003; C07D 257/02; C07B 2200/09

(57) ABSTRACT

The invention relates to compounds of formula (I) and to their use in treating or preventing an inflammatory disease or a disease associated with an undesirable immune response:

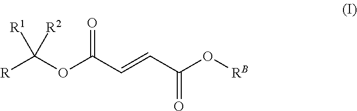

wherein R, R¹, R² and R^{B} are as defined herein.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Brück J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKCθ with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1α (HIF-1α) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

US 2020/0000758 discloses a method of treating psoriasis with sustained release compression coated tablet dosage forms comprising certain methyl hydrogen fumarate prodrugs. WO 2018/191221 discloses GHB (gamma-hydroxybutyrate) prodrug fumarates which are said to decrease or deter the potential for GHB abuse, illicit and illegal use, and overdose. WO 2018/183264 also discloses fumarates which are said to decrease or deter the potential for opioid abuse, addiction, illicit and illegal use, and overdose. WO 2016/061393 discloses monomethyl and monoethyl fumarate prodrugs which are said to have utility in the treatment of neurodegenerative, inflammatory and autoimmune disorders.

In spite of the above findings, there remains a need to identify and develop new therapeutics possessing enhanced properties compared to currently marketed anti-inflammatory agents, such as DMF. The present inventors have now discovered novel fumarate compounds which are more effective at reducing cytokine release in cells and/or in activating NRF2-driven effects than dimethyl fumarate. These properties, amongst others, including enhanced metabolic and hydrolytic stability, make them potentially more effective than dimethyl fumarate and/or diroximel fumarate (WO 2014/152494; Naismith R. T. et al., CNS Drugs 2020, 34, 185-196). Such compounds therefore possess excellent anti-inflammatory properties.

### SUMMARY OF THE INVENTION

The present invention provides a compound of formula (I):

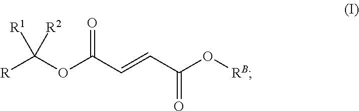

wherein:
R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₄₋₁₀ cycloalkyl ring, wherein the C₄₋₁₀ cycloalkyl ring is optionally substituted by one or more R^{c} wherein R^{c} is independently selected from the group consisting of halo, C₁₋₂ alkyl, C₁₋₂ haloalkyl, C₁₋₂ alkoxy and C₁₋₂ haloalkoxy, and/or wherein the C₄₋₁₀ cycloalkyl ring is optionally substituted by two R^{c} groups wherein the two R^{C} groups are attached to the same carbon atom and are joined to form a C₄₋₆ cycloalkyl ring; and
R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl, and/or wherein R^{B} is optionally substituted by two R^{B′} groups, attached to the same carbon atom, that are joined to form a C₃₋₆ cycloalkyl or a 4-6-membered heterocyclic ring;
wherein the total number of carbon atoms in groups R, R¹ and R² taken together, including their optional substituents, and including the carbon to which R, R¹ and R² are attached, is 6 to 14;

or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an undesirable immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

### Compounds of Formula (I)

Embodiments and preferences set out herein with respect to the compound of formula (I) apply equally to the pharmaceutical composition, compound for use, use and method aspects of the invention.

As used herein, the term “alkyl”, such as “C₄₋₁₀ alkyl”, “C₁₋₄ alkyl” or “C₁₋₂ alkyl”, refers to a straight or branched fully saturated hydrocarbon group having the specified number of carbon atoms. The term encompasses methyl, ethyl, n-propyl, iso-propyl, n-butyl, iso-butyl, sec-butyl, tert-butyl, n-pentyl, n-hexyl, n-heptyl, n-octyl, n-nonyl and n-decyl. Branched variants are also included such as (n-Bu)₂CH—, n-pentyl-CH(CH₂CH₃)—, n-pentyl-C(CH₃)₂—, n-hexyl-C(CH₃)₂— and n-heptyl-CH(CH₃)—. The term “alkyl” also encompasses “alkylene” which is a bifunctional straight or branched fully saturated hydrocarbon group having the stated number of carbon atoms. Example “alkylene” groups include methylene, ethylene, n-propylene, n-butylene, n-pentylene, n-heptylene, n-hexylene and n-octylene.

The term “cycloalkyl”, such as “C₆₋₁₀ cycloalkyl” or “C₃₋₆ cycloalkyl”, refers to a fully saturated cyclic hydrocarbon group having the specified number of carbon atoms. The term encompasses cyclopropyl, cyclobutyl, cyclopentyl, cyclohexyl, cycloheptyl, cyclooctyl, cyclononyl and cyclodecyl as well as bridged systems such as bicyclo[1.1.1]pentyl, bicyclo[2.2.1]heptyl, bicyclo[2.2.2]octyl and adamantyl.

The term “haloalkyl”, such as “C₁₋₃ haloalkyl”, “C₁₋₂ haloalkyl” or “C₁ haloalkyl”, refers to a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂, CF₂CH₃ and CH₂CF₃.

The term “haloalkoxy” refers to a haloalkyl group, such as “C₁₋₃ haloalkyl”, “C₁₋₂ haloalkyl” or “C₁ haloalkyl”, as defined above, singularly bonded via an oxygen atom. Examples of haloalkoxy groups include OCF₃, OCHF₂ and OCH₂CF₃.

The term “halo” refers to fluorine, chlorine, bromine or iodine. Particular examples of halo are fluorine, chlorine and bromine, especially fluorine.

The term “5- or 6-membered heteroaryl” refers to a cyclic group with aromatic character containing the indicated number of atoms (5 or 6) wherein at least one of the atoms in the cyclic group is a heteroatom independently selected from N, O and S. The term encompasses pyrrolyl, furanyl, thienyl, imidazolyl, pyrazolyl, thiazolyl, oxadiazolyl, thiadiazolyl, triazolyl, oxazolyl, tetrazolyl, pyridyl, pyrimidinyl, pyradizinyl and pyrazinyl.

The term “tetrazolyl” refers to a 5-(1H-tetrazolyl) substituent where the tetrazole is linked to the rest of the molecule via a carbon atom:

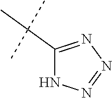

wherein the dashed line indicates the point of attachment to the rest of the molecule.

The term “4-6-membered heterocyclic ring” refers to a non-aromatic cyclic group having 4 to 6 ring atoms and wherein at least one of the ring atoms is a heteroatom selected from N, O, S and B. The term “heterocyclic ring” is interchangeable with “heterocyclyl”. The term encompasses oxetanyl, thietanyl, azetidinyl, pyrrolidinyl, tetrahydrofuranyl, tetrahydrothienyl, tetrahydropyranyl, piperidinyl, piperazinyl, morpholinyl and thiomorpholinyl. 4-6-membered heterocyclyl groups can typically be substituted by one or more (e.g. one or two) oxo groups. Suitably, thietanyl is substituted by one or two oxo groups.

Where substituents are indicated as being optionally substituted in formula (I) in the embodiments and preferences set out below, the optional substituent may be attached to an available carbon atom, which means a carbon atom which is attached to a hydrogen atom i.e. a C—H group. The optional substituent replaces the hydrogen atom attached to the carbon atom.

In one embodiment, the invention provides a compound of formula (I):

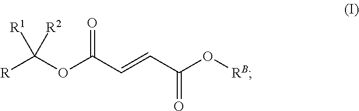

wherein:
R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₆₋₁₀ cycloalkyl and phenyl, and R¹ and R² are independently selected from the group consisting of H, C₁₋₄ alkyl and C₁₋₄ haloalkyl, or R¹ and R² join to form a C₃₋₄ cycloalkyl ring; wherein R is optionally substituted by one or more R^{b} wherein R^{b} is independently selected from the group consisting of halo, C₁₋₄ alkyl, C₁₋₄ haloalkyl, C₁₋₄ alkoxy and C₁₋₄ haloalkoxy; or
R is H, methyl or CF₃ and R¹ and R² are joined to form a C₄₋₁₀ cycloalkyl ring, wherein the C₄₋₁₀ cycloalkyl ring is optionally substituted by one or more R^{c} wherein R^{c} is independently selected from the group consisting of halo, C₁₋₂ alkyl, C₁₋₂ haloalkyl, C₁₋₂ alkoxy and C₁₋₂ haloalkoxy, and/or wherein the C₄₋₁₀ cycloalkyl ring is optionally substituted by two R^{c} groups wherein the two R^{C} groups are attached to the same carbon atom and are joined to form a C₄₋₆ cycloalkyl ring; and
R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl, and/or wherein R^{B} is optionally substituted by two R^{B′} groups, attached to the same carbon atom, that are joined to form a C₃₋₆ cycloalkyl or a 4-6-membered heterocyclic ring;
wherein the total number of carbon atoms in groups R, R¹ and R² taken together, including their optional substituents, and including the carbon to which R, R¹ and R² are attached, is 6 to 14;

or a pharmaceutically acceptable salt and/or solvate thereof.

In one embodiment, R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring.

In one embodiment, R is C₄ alkyl. In another embodiment, R is C₅ alkyl. In another embodiment, R is C₆ alkyl. In another embodiment, R is C₇ alkyl. In another embodiment, R is C₈ alkyl. In another embodiment, R is C₉ alkyl. In another embodiment, R is C₁₀ alkyl. Most suitably, R is C₇ alkyl.

Suitably, the C₇ alkyl group is linear such that the following group forms:

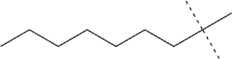

wherein the dashed bond indicates the point of attachment to the C atom attached to R¹ and R².

In one embodiment, R¹ is H. In another embodiment, R¹ is C₁₋₄ alkyl such as methyl. In another embodiment, R¹ is C₁₋₄ haloalkyl such as CF₃.

In one embodiment, R² is H. In another embodiment, R² is C₁₋₄ alkyl such as methyl. In another embodiment, R² is C₁₋₄ haloalkyl such as CF₃.

In one embodiment, R¹ and R² join to form a C₃₋₄ cycloalkyl ring. Suitably, R¹ and R² join to form a C₃ cycloalkyl ring. Alternatively, R¹ and R² join to form a C₄ cycloalkyl ring.

Suitably, R¹ is CF₃ and R² is H. Alternatively, R¹ is methyl and R² is methyl. Most suitably, R¹ is methyl and R² is H.

When R¹ and R² are different, suitably the groups have the following configuration:

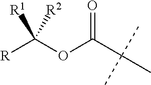

wherein the dashed line indicates the point of attachment to the rest of the molecule.

In one embodiment, R is not substituted. In another embodiment, R is substituted by one or more R^{a}. In one embodiment, R is substituted by one R^{a} group. In another embodiment, R is substituted by two R^{a} groups. In another embodiment, R is substituted by three R^{a} groups. In another embodiment, R is substituted by four R^{a} groups.

In one embodiment, R^{a} is halo such as fluoro. In another embodiment, R^{a} is C₁₋₂ haloalkyl such as CF₃. In another embodiment, R^{a} is C₁₋₂ haloalkoxy such as OCF₃.

In another embodiment, R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano.

Suitably, R is selected from the group consisting of C₆₋₁₀ cycloalkyl and phenyl, and R¹ and R² are independently selected from the group consisting of H, C₁₋₄ alkyl and C₁₋₄ haloalkyl, or R¹ and R² join to form a C₃₋₄ cycloalkyl ring.

In one embodiment, R is C₆₋₁₀ cycloalkyl such as C₆₋₈ cycloalkyl. Suitably, R is C₆ cycloalkyl. Alternatively, R is C₇ cycloalkyl. Alternatively, R is C₈ cycloalkyl. Alternatively, R is C₉ cycloalkyl. Alternatively, R is C₁₀ cycloalkyl.

In another embodiment, R is phenyl.

In another embodiment, R is 5- or 6-membered heteroaryl.

In one embodiment, R¹ is H. In another embodiment, R¹ is C₁₋₄ alkyl such as methyl. In another embodiment, R¹ is C₁₋₄ haloalkyl such as CF₃.

In one embodiment, R² is H. In another embodiment, R² is C₁₋₄ alkyl such as methyl. In another embodiment, R² is C₁₋₄ haloalkyl such as CF₃.

In one embodiment, R¹ and R² join to form a C₃₋₄ cycloalkyl ring. Suitably, R¹ and R² join to form a C₃ cycloalkyl ring. Alternatively, R¹ and R² join to form a C₄ cycloalkyl ring.

In an embodiment, the C₃₋₄ cycloalkyl ring is not substituted. In another embodiment, the C₃₋₄ cycloalkyl ring is substituted by methyl, halo or cyano.

In another embodiment, R¹ and R² join to form a 4-6-membered heterocyclic ring. In one embodiment, R¹ and R² join to form a 4-membered heterocyclic ring such as oxetanyl or thietanyl. In another embodiment, R¹ and R² join to form a 5-membered heterocyclic ring. In another embodiment, R¹ and R² join to form a 6-membered heterocyclic ring.

Suitably, R¹ is CF₃ and R² is H. Alternatively, R¹ is methyl and R² is methyl. Most suitably, R¹ is methyl and R² is H.

When R¹ and R² are different, suitably the groups have the following configuration:

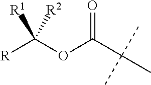

wherein the dashed line indicates the point of attachment to the rest of the molecule.

In one embodiment, R is not substituted. In another embodiment, R is substituted by one or more R^{b}. In one embodiment, R is substituted by one R^{b} group. In another embodiment, R is substituted by two R^{b} groups. In another embodiment, R is substituted by three R^{b} groups. In another embodiment, R is substituted by four R^{b} groups.

In one embodiment, R^{b} is halo such as chloro or bromo. In another embodiment, R^{b} is C₁₋₄ alkyl such as methyl. In another embodiment, R^{b} is C₁₋₄ haloalkyl such as CF₃. In another embodiment, R^{b} is C₁₋₄ alkoxy such as OCH₃. In another embodiment, R^{b} is C₁₋₄ haloalkoxy, such as OCF₃. In another embodiment, R^{b} is cyano.

Suitably, when R¹ and R² join to form a C₃ cycloalkyl ring, R is phenyl and is substituted by one R^{b} wherein R^{b} is halo, e.g., bromo.

Suitably, when R¹ and R² join to form a C₄ cycloalkyl ring, R is phenyl and is substituted by two R^{b} wherein R^{b} is halo, e.g., chloro.

In one embodiment, R is H, methyl or CF₃ and R¹ and R² are joined to form a C₄₋₁₀ cycloalkyl ring. Suitably, R is H. Alternatively, R is methyl. Alternatively, R is CF₃. Most suitably, R is H.

In this embodiment, R¹ and R² are joined to form a C₄₋₁₀ cycloalkyl ring such as a C₆₋₈ cycloalkyl ring. In one embodiment, R¹ and R² are joined to form a C₄ cycloalkyl ring. In another embodiment, R¹ and R² are joined to form a C₅ cycloalkyl ring. In another embodiment, R¹ and R² are joined to form a C₆ cycloalkyl ring. In another embodiment, R¹ and R² are joined to form a C₇ cycloalkyl ring. In another embodiment, R¹ and R² are joined to form a C₈ cycloalkyl ring. In another embodiment, R¹ and R² are joined to form a C₉ cycloalkyl ring. In another embodiment, R¹ and R² are joined to form a C₁₀ cycloalkyl ring. Most suitably, R¹ and R² are joined to form a C cycloalkyl ring.

In one embodiment, the C₄₋₁₀ cycloalkyl ring is not substituted. In another embodiment, the C₄₋₁₀ cycloalkyl ring is substituted by one or more R^{c}. In one embodiment, the C₄₋₁₀ cycloalkyl ring is substituted by one R^{c} group. In another embodiment, the C₄₋₁₀ cycloalkyl ring is substituted by two R^{c} groups. In another embodiment, the C₄₋₁₀ cycloalkyl ring is substituted by three R^{c} groups. In another embodiment, the C₄₋₁₀ cycloalkyl ring is substituted by four R^{c} groups.

In one embodiment, R^{c} is halo such as fluoro. In another embodiment, R^{c} is C₁₋₂ alkyl such as methyl. In another embodiment, R^{c} is C₁₋₂ haloalkyl such as CF₃. In another embodiment, R^{c} is C₁₋₂ alkoxy such as methoxy. In another embodiment, R^{c} is C₁₋₂ haloalkoxy such as OCF₃.

In one embodiment, C₄₋₁₀ cycloalkyl ring is substituted by two R^{c} groups wherein the two R^{C} groups are attached to the same carbon atom and are joined to form a C₄₋₆ cycloalkyl ring. Suitably, the two R^{C} groups join to form a C₄ cycloalkyl ring. Alternatively, the two R^{C} groups join to form a C₅ cycloalkyl ring. Alternatively, the two R^{C} groups join to form a C₆ cycloalkyl ring.

Most suitably, R¹ and R² are joined to form a C₄ cycloalkyl ring substituted by two R^{C} groups which are attached to the same carbon atom and are joined to form a C₄ cycloalkyl ring. In this embodiment, suitably R is H.

Suitably, the two R^{c} groups are attached to the 3-position of the C₄ cycloalkyl ring so that the following moiety forms:

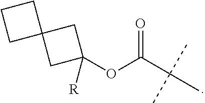

In any of the above embodiments, and unless otherwise stated, the substituent groups R^{a}, R^{b} and R^{c} may be attached to the same carbon atom, or may be attached to different carbon atoms.

The total number of carbon atoms in groups R, R¹ and R² taken together, including their optional substituents, and including the carbon to which R, R¹ and R² are attached, is 6 to 14. In one embodiment, the total number of carbon atoms is 6 carbon atoms. In another embodiment, the total number of carbon atoms is 7 carbon atoms. In another embodiment, the total number of carbon atoms is 8 carbon atoms. In another embodiment, the total number of carbon atoms is 9 carbon atoms. In another embodiment, the total number of carbon atoms is 10 carbon atoms. In another embodiment, the total number of carbon atoms is 11 carbon atoms. In another embodiment, the total number of carbon atoms is 12 carbon atoms. In another embodiment, the total number of carbon atoms is 13 carbon atoms. In another embodiment, the total number of carbon atoms is 14 carbon atoms.

In one embodiment, R^{B} is CH₂COOH. In another embodiment, R^{B} is CH₂CH₂COOH. In another embodiment, R^{B} is CH₂tetrazolyl. In another embodiment, R^{B} is CH₂CH₂tetrazolyl. Suitably, R^{B} is CH₂COOH or CH₂CH₂COOH.

In one embodiment, R^{B} is not substituted.

In another embodiment, R^{B} is substituted on an available carbon atom by one or more such as one, two, three or four, e.g., one R^{B′} wherein R^{B′} is selected from the group consisting of difluoromethyl, trifluoromethyl and methyl, and/or wherein R^{B} is optionally substituted by two R^{B′} groups, attached to the same carbon atom, that are joined to form a C₃₋₆ cycloalkyl or a 4-6-membered heterocyclic ring.

In one embodiment, R^{B} is substituted by one R^{B′}. In another embodiment, R^{B} is substituted by two R^{B′}. In another embodiment, R^{B} is substituted by three R^{B′}. In another embodiment, R^{B} is substituted by four R^{B′}.

In one embodiment, R^{B′} is difluoromethyl. In another embodiment, R^{B′} is trifluoromethyl. In another embodiment, R^{B′} is methyl. Suitably, R^{B} is substituted by one methyl group. Alternatively, R^{B} is substituted by two R^{B′} groups, attached to the same carbon atom, that are joined to form a C₃₋₆ cycloalkyl or a 4-6-membered heterocyclic ring. Suitably, the two R^{B′} groups join to form a C₃₋₆ cycloalkyl ring such as a C₃ cycloalkyl ring. Alternatively, the two R^{B′} groups join to form a 4-6-membered heterocyclic ring.

Suitably, R^{B′} is attached to the same or different carbon to the carbon attached to the COOH or tetrazolyl group. When R^{B} is CH₂CH₂COOH or CH₂CH₂tetrazolyl, suitably R^{B′} is attached to the carbon atom linked to the oxygen atom of the carboxylate group attached to R^{B}.

Suitably, the two R^{B′} groups, attached to the same carbon atom, that are joined to form a C₃₋₆ cycloalkyl or a 4-6-membered heterocyclic ring are attached to the same or different carbon to the carbon attached to the COOH or tetrazolyl group. When R^{B} is CH₂CH₂COOH or CH₂CH₂tetrazolyl, suitably the two R^{B′} groups are attached to the carbon atom linked to the oxygen atom of the carboxylate group attached to R^{B}.

In one embodiment, the molecular weight of the compound of formula (I) is 150 Da-450 Da, suitably 200 Da-400 Da.

In one embodiment there is provided a compound of formula (I), selected from the group consisting of:
(E)-2-((4-(cyclooctyloxy)-4-oxobut-2-enoyl)oxy)acetic acid;
(E)-2-((4-(cyclohexyloxy)-4-oxobut-2-enoyl)oxy)acetic acid;
(E)-3-((4-(Cyclooctyloxy)-4-oxobut-2-enoyl)oxy)propanoic acid;
(E)-3-((4-(cyclohexyloxy)-4-oxobut-2-enoyl)oxy)propanoic acid;
2-(1H-tetrazol-5-yl)ethyl cyclooctyl fumarate;
(S,E)-2-((4-(cyclooctyloxy)-4-oxobut-2-enoyl)oxy)propanoic acid;
(E)-3-((4-(cyclooctyloxy)-4-oxobut-2-enoyl)oxy)-2,2-dimethylpropanoic acid;
(E)-1-((4-(cyclooctyloxy)-4-oxobut-2-enoyl)oxy)cyclopropane-1-carboxylic acid;
(E)-2-((4-oxo-4-(spiro[3.3]heptan-2-yloxy)but-2-enoyl)oxy)acetic acid;
(E)-2-((4-(cycloheptyloxy)-4-oxobut-2-enoyl)oxy)acetic acid;
(E)-3-((4-(cyclooctyloxy)-4-oxobut-2-enoyl)oxy)butanoic acid;
(R,E)-2-((4-(cyclooctyloxy)-4-oxobut-2-enoyl)oxy)propanoic acid;
2-[(E)-4-[(1R)-1-methylheptoxy]-4-oxo-but-2-enoyl]oxyacetic acid; and
(R,E)-3-((4-(octan-2-yloxy)-4-oxobut-2-enoyl)oxy)propanoic acid;

or a pharmaceutically acceptable salt and/or solvate of any one thereof.

In another embodiment there is provided a compound of formula (I) which is:
(E)-2-((4-oxo-4-(1-(4-(trifluoromethyl)phenyl)cyclobutoxy)but-2-enoyl)oxy) acetic acid;

or a pharmaceutically acceptable salt and/or solvate of any one thereof.

In another embodiment, there is provided a compound of formula (I) which is:
(E)-3-(4-oxo-4-(1-(4-(trifluoromethyl)phenyl)cyclobutoxy)but-2-enoyloxy)propanoic acid;

or a pharmaceutically acceptable salt and/or solvate of any one thereof.

Compounds of formula (I) may be prepared as set out in the Examples and as set out in the following schemes. As used herein, R^{A} is equivalent to the following group:

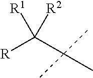

wherein R, R¹ and R² are defined elsewhere herein and the dashed line indicates the connection to the remainder of the compound of formula (I).

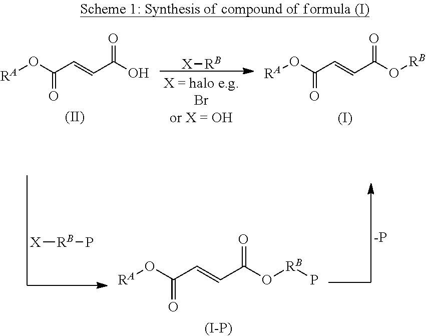

Compounds of formula (I) may be prepared from compounds of formula (II) under standard ester forming conditions which are well known to the person skilled in the art. For example, when X=halo, such as Br, compounds of formula (I) can be prepared from compounds of formula (II) using X—R^{B} in the presence of base e.g. K₂CO₃ in solvent such as acetone. When X═OH, compounds of formula (I) may be accessed via condensation reactions employing a coupling agent e.g. EDCI/DMAP in presence of a base e.g. DIPEA in a solvent such as DCM. Alternatively, when X ═OH, the carboxyl group may be activated with an activating agent such as (COCl)₂ in a solvent, e.g., a dimethylformamide/DCM mixture, following by addition of a base e.g. Et₃N in a solvent, e.g., DCM, to provide compounds of formula (I).

Compounds of formula (II) may be reacted with a protected derivative of X—R^{B} such as X—R^{B}—P, wherein P is a carboxylic acid protecting group such as C₁₋₆ alkyl e.g. tert-butyl, or para-methoxybenzyl (Scheme 1). In such instances, the protecting group may be removed as the final step using conditions known to the person skilled in the art. For example, a carboxylic acid protecting group such as C₁₋₆ alkyl e.g. tert-butyl, or para-methoxybenzyl may be removed under acidic conditions such as TFA in DCM.

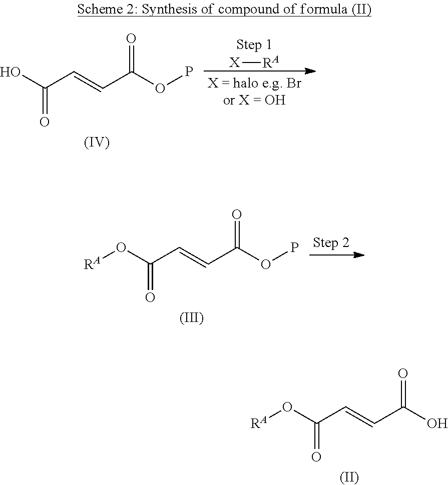

Compounds of formula (II) may be prepared from compounds of formula (IV), wherein P is a carboxylic acid protecting group such as C₁₋₆ alkyl e.g. tert-butyl, or para-methoxybenzyl. P may also be Fmoc.

Step 1: When X=halo such as Br, compounds of formula (III) can be prepared from compounds of formula (IV) using X—R^{A} in the presence of base e.g. K₂CO₃ in solvent such as acetone. When X═OH, compounds of formula (III) may be accessed via condensation reactions employing a coupling agent e.g. EDCI/DMAP in presence of a base e.g. DIPEA in a solvent such as DCM. Alternatively, when X═OH, the carboxyl group may be activated with an activating agent such as (COCl)₂ in a solvent e.g. a dimethylformamide/DCM mixture, following by addition of a base e.g. Et₃N in a solvent e.g. DCM to give compounds of formula (III).

Step 2: Compounds of formula (II) may be obtained by removal of protecting group P using conditions known to the person skilled in the art. For example, when P is C₁₋₆ alkyl e.g. tert-butyl, or para-methoxybenzyl P may be removed under acidic conditions such as TFA in DCM. When P is Fmoc, the protecting group may be removed using basic conditions such as TEA in dimethylformamide.

The skilled person will appreciate that protecting groups may be used throughout the synthetic schemes described herein to give protected derivatives of any of the above compounds or generic formulae. Protective groups and the means for their removal are described in “Protective Groups in Organic Synthesis”, by Theodora W. Greene and Peter G. M. Wuts, published by John Wiley & Sons Inc; 4th Rev Ed., 2006, ISBN-10: 0471697540. Examples of nitrogen protecting groups include trityl (Tr), tert-butyloxycarbonyl (BOC), 9-fluorenylmethyloxycarbonyl (Fmoc), acetyl (Ac), benzyl (Bn) and para-methoxybenzyl (PMB). Examples of oxygen protecting groups include acetyl (Ac), methoxymethyl (MOM), para-methoxybenzyl (PMB), benzyl, tert-butyl, methyl, ethyl, tetrahydropyranyl (THP), and silyl ethers and esters (such as trimethylsilyl (TMS), tert-butyldimethylsilyl (TBDMS), tri-iso-propylsilyloxymethyl (TOM), and triisopropylsilyl (TIPS) ethers and esters). Specific examples of carboxylic acid protecting groups include alkyl esters (such as C₁₋₆ alkyl e.g. C₁₋₄ alkyl esters), benzyl esters and silyl esters. Specific examples of carboxylic acid protecting groups include alkyl esters (such as C₁₋₆ alkyl e.g. C₁₋₄ alkyl esters), benzyl esters (e.g. para-methoxybenzyl) and silyl esters.

In one embodiment, there is provided a process for the preparation of compounds of formula (I) or a salt, such as a pharmaceutically acceptable salt thereof, which comprises reacting a compound of formula (II):

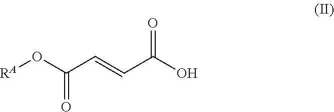

or a salt such as a pharmaceutically acceptable salt thereof, wherein R^{A} is defined elsewhere herein;

with X—R^{B} or a salt, such as a pharmaceutically acceptable salt thereof, wherein X is halo e.g. Br, or OH, and R^{B} is defined elsewhere herein.

In another embodiment, there is provided a process for the preparation of compounds of formula (I) or a salt, such as a pharmaceutically acceptable salt thereof, which comprises reacting a compound of formula (II):

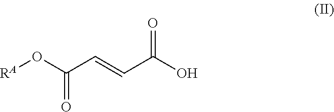

or a salt such as a pharmaceutically acceptable salt thereof, wherein R^{A} is defined elsewhere herein;

with X—R^{B}—P or a salt, such as a pharmaceutically acceptable salt thereof, followed by removal of protecting group P, wherein P is a carboxylic acid protecting group such as C₁₋₆ alkyl e.g. tert-butyl, or para-methoxybenzyl, X is halo e.g. Br, or OH, and R^{B} is defined elsewhere herein.

Protecting group P may be removed under conditions known to the skilled person. When P is C₁₋₆ alkyl, e.g., tert-butyl, P may be removed using acidic conditions such as TFA in DCM. When P is para-methoxybenzyl, P may also be removed using acidic conditions, such as hydrogen chloride in dioxane.

In one embodiment, there is provided a compound of formula (I-P):

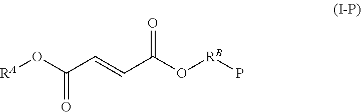

or a salt such as a pharmaceutically acceptable salt thereof, wherein R^{A}, R^{B} and P are defined elsewhere herein.

In one embodiment, there is provided a compound of formula (II):

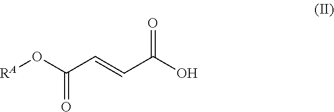

or a salt such as a pharmaceutically acceptable salt thereof, wherein R^{A} is defined elsewhere herein.

Suitably, the compound of formula (II) is other than 1-octyl fumarate and (E)-4-(cycloheptyloxy)-4-oxobut-2-enoic acid.

In one embodiment, there is provided a compound of formula (III):

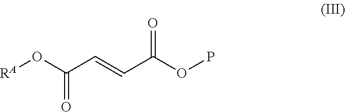

or a salt such as a pharmaceutically acceptable salt thereof, wherein R^{A} is defined elsewhere herein and P is a carboxylic acid protecting group such as C₁₋₆ alkyl e.g. tert-butyl, or para-methoxybenzyl.

The moiety “—R^{B}—P” as used herein means that R^{B} is protected with protecting group P. The location and specific protecting group will depend on the identity of R^{B} which will be understood by the skilled person.

For example, when R^{B} comprises CH₂COOH or CH₂CH₂COOH, suitably P is a carboxylic acid protecting group and suitably replaces the hydrogen atom attached to an oxygen atom, i.e., CH₂COO—P or CH₂CH₂COO—P.

When R^{B} comprises CH₂tetrazolyl or CH₂CH₂tetrazolyl, suitably P is a tetrazolyl protecting group which replaces the hydrogen atom attached to a nitrogen atom:

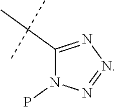

Certain intermediates are novel and are claimed as an aspect of the invention:
2-(tert-butoxy)-2-oxoethyl cyclooctyl fumarate;
2-(tert-butoxy)-2-oxoethyl cyclohexyl fumarate;
3-(tert-butoxy)-3-oxopropyl cyclooctyl fumarate;
3-(tert-butoxy)-3-oxopropyl cyclohexyl fumarate;
(S)-1-(tert-butoxy)-1-oxopropan-2-yl cyclooctyl fumarate;
Cyclooctyl (3-((4-methoxybenzyl)oxy)-2,2-dimethyl-3-oxopropyl) fumarate;
2-(tert-butoxy)-2-oxoethyl spiro[3.3]heptan-2-yl fumarate;
2-(tert-butoxy)-2-oxoethyl cycloheptyl fumarate;
Cyclooctyl (4-((4-methoxybenzyl)oxy)-4-oxobutan-2-yl) fumarate;
(R)-1-(tert-butoxy)-1-oxopropan-2-yl cyclooctyl fumarate;
(R)-2-(tert-butoxy)-2-oxoethyl octan-2-yl fumarate;
(R)-3-(tert-butoxy)-3-oxopropyl octan-2-yl fumarate;

or a salt thereof.

Such intermediates may be considered prodrugs of compounds of formula (I).

Also provided is a compound selected from the group consisting of:
(E)-4-oxo-4-(1-(4-(trifluoromethyl)phenyl)cyclobutoxy)but-2-enoic acid;
(E)-4-(1-methylcyclobutoxy)-4-oxobut-2-enoic acid;
octyl fumarate;
(E)-4-(cyclooctyloxy)-4-oxobut-2-enoic acid;
(E)-4-oxo-4-(spiro[3.3]heptan-2-yloxy)but-2-enoic acid; and
(E)-4-(cycloheptyloxy)-4-oxobut-2-enoic acid;

or a salt, such as a pharmaceutically acceptable salt, thereof.

Suitably, there is provided a compound selected from the group consisting of:
(E)-4-oxo-4-(1-(4-(trifluoromethyl)phenyl)cyclobutoxy)but-2-enoic acid;
(E)-4-(1-methylcyclobutoxy)-4-oxobut-2-enoic acid;
(E)-4-(cyclooctyloxy)-4-oxobut-2-enoic acid; and
(E)-4-oxo-4-(spiro[3.3]heptan-2-yloxy)but-2-enoic acid;

or a salt, such as a pharmaceutically acceptable salt, thereof.

Also provided is a compound selected from the group consisting of:
(E)-4-oxo-4-(1-(5-(trifluoromethyl)pyridin-2-yl)cyclobutoxy)but-2-enoic acid;
(E)-4-oxo-4-(1-(3-(trifluoromethyl)phenyl)cyclobutoxy)but-2-enoic acid;
(E)-4-oxo-4-(1-(2-(trifluoromethyl)phenyl)cyclobutoxy)but-2-enoic acid;
(E)-4-(1-(4-bromophenyl)cyclobutoxy)-4-oxobut-2-enoic acid;
(E)-4-(1-(4-chlorophenyl)cyclobutoxy)-4-oxobut-2-enoic acid;
(E)-4-(1-(3,5-dichlorophenyl)cyclobutoxy)-4-oxobut-2-enoic acid;
(E)-4-oxo-4-(1-(6-(trifluoromethyl)pyridin-3-yl)cyclobutoxy)but-2-enoic acid;
(E)-4-(1-(3-fluoro-4-(trifluoromethyl)phenyl)cyclobutoxy)-4-oxobut-2-enoic acid;
(E)-4-oxo-4-((3-(4-(trifluoromethyl)phenyl)thietan-3-yl)oxy)but-2-enoic acid;
(E)-4-oxo-4-((3-(4-(trifluoromethyl)phenyl)oxetan-3-yl)oxy)but-2-enoic acid;
(S,E)-4-oxo-4-(1-(4-(trifluoromethyl)phenyl)ethoxy)but-2-enoic acid;
(R,E)-4-oxo-4-(1-(4-(trifluoromethyl)phenyl)ethoxy)but-2-enoic acid:
(E)-4-oxo-4-((2-(4-(trifluoromethyl)phenyl)propan-2-yl)oxy)but-2-enoic acid;
(E)-4-(1-(5-bromopyridin-2-yl)cyclobutoxy)-4-oxobut-2-enoic acid;
(E)-4-(1-(5-chloropyridin-2-yl)cyclobutoxy)-4-oxobut-2-enoic acid;
(E)-4-(1-(3,5-dichloro-4-fluorophenyl)cyclobutoxy)-4-oxobut-2-enoic acid;
(E)-4-(1-(3-chloro-4-(trifluoromethyl)phenyl)cyclobutoxy)-4-oxobut-2-enoic acid;
(E)-4-(1-(4-cyanophenyl)cyclobutoxy)-4-oxobut-2-enoic acid;
(E)-4-oxo-4-(1-(3,4,5-trifluorophenyl)cyclobutoxy)but-2-enoic acid;
(E)-4-(3-methyl-1-(4-(trifluoromethyl) phenyl)cyclobutoxy)-4-oxobut-2-enoic acid;
(E)-4-oxo-4-((4-(4-(trifluoromethyl) phenyl)tetrahydro-2H-pyran-4-yl)oxy)but-2-enoic acid;
(E)-4-(3-cyano-1-(4-(trifluoromethyl) phenyl)cyclobutoxy)-4-oxobut-2-enoic acid;
(E)-4-oxo-4-(1-(5-(trifluoromethyl)thiophen-2-yl)cyclobutoxy)but-2-enoic acid;
(E)-4-(1-(3,5-difluoro-4-(trifluoromethyl) phenyl)cyclobutoxy)-4-oxobut-2-enoic acid;
(E)-4-oxo-4-(1-(4-(trifluoromethoxy)phenyl)cyclobutoxy)but-2-enoic acid;
(E)-4-(3,3-difluoro-1-(4-(trifluoromethyl) phenyl)cyclobutoxy)-4-oxobut-2-enoic acid; and
(E)-4-(1-(4-(difluoromethyl)phenyl)cyclobutoxy)-4-oxobut-2-enoic acid;

or a salt, such as a pharmaceutically acceptable salt, thereof.

There is also provided a compound selected from the group consisting of:
(E)-4-oxo-4-(1-(4-(trifluoromethyl)phenyl)cyclopropoxy)but-2-enoic acid;
(E)-4-oxo-4-(1-(5-(trifluoromethyl)pyrimidin-2-yl)cyclobutoxy)but-2-enoic acid;
(E)-4-(1-(3,5-dimethoxyphenyl)cyclobutoxy)-4-oxobut-2-enoic acid;
(E)-4-(1-(3-chloro-5-(trifluoromethoxy)phenyl)cyclobutoxy)-4-oxobut-2-enoic acid;
(E)-4-oxo-4-(2,2,2-trifluoro-1-(4-(trifluoromethyl)phenyl)ethoxy)but-2-enoic acid; and
(E)-4-oxo-4-(2,2,2-trifluoro-1-(4-(trifluoromethyl)phenyl)ethoxy)but-2-enoic acid;

or a salt, such as a pharmaceutically acceptable salt, thereof.

Suitably, the compound is:
(E)-4-oxo-4-(1-(4-(trifluoromethyl)phenyl)cyclobutoxy)but-2-enoic acid;

or a salt, such as a pharmaceutically acceptable salt, thereof.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids, e.g., hydrochloric acid, hydrobromic acid, sulfuric acid, nitric acid or phosphoric acid. Also included are salts formed with organic acids e.g. succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts, e.g., oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines e.g. with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt e.g. is not a pharmaceutically acceptable salt.

Compounds of formula (II) may be in the form of a salt, such as a pharmaceutically acceptable salt, such as those defined above. Suitably, the compound of formula (II) is not a salt, e.g., is not a pharmaceutically acceptable salt.

Suitably, where the compound of formula (I) or the compound of formula (II) is in the form of a salt, the pharmaceutically acceptable salt is a basic addition salt such as a carboxylate salt formed with a group 1 metal (e.g. a sodium or potassium salt), a group 2 metal (e.g. a magnesium or calcium salt) or an ammonium salt of a basic amine (e.g. an NH₄⁺ salt), such as a sodium salt.

The compounds of formula (I) may be prepared in crystalline or non-crystalline form and, if crystalline, may optionally be solvated, e.g., as the hydrate. This invention includes within its scope stoichiometric solvates (e.g., hydrates) as well as compounds containing variable amounts of solvent (e.g., water). Suitably, the compound of formula (I) is not a solvate.

The compounds of formula (II) may be prepared in crystalline or non-crystalline form and, if crystalline, may optionally be solvated, e.g., as the hydrate. This invention includes within its scope stoichiometric solvates (e.g., hydrates) as well as compounds containing variable amounts of solvent (e.g., water). Suitably, the compound of formula (II) is not a solvate.

The invention extends to a pharmaceutically acceptable derivative thereof, such as a pharmaceutically acceptable prodrug of compounds of formula (I). The invention also extends to a pharmaceutically acceptable derivative of compounds of formula (II), such as a pharmaceutically acceptable prodrug of compounds of formula (II). Typical prodrugs of compounds of formula (I) which comprise a carboxylic acid, and compounds of formula (II), include ester (e.g. C₁₋₆ alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug. In one embodiment, the compound of formula (II) is provided as a pharmaceutically acceptable prodrug. In another embodiment, the compound of formula (II) is not provided as a pharmaceutically acceptable prodrug.

Certain compounds of formula (I) may metabolise under certain conditions such as by hydrolysis of the R^{B} ester group. Without wishing to be bound by theory, formation of an active metabolite (such as in vivo) of a compound of formula (I) may be beneficial by contributing to the biological activity observed of the compound of formula (I). Thus, in one embodiment, there is provided an active metabolite of the compound of formula (I) and its use as a pharmaceutical e.g. for the treatment or prevention of the diseases mentioned herein.

It is to be understood that the present invention encompasses all isomers of compounds of formula (I) including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also encompasses all isomers of compounds of formula (II) including all geometric, tautomeric and optical forms, and mixtures thereof (e.g., racemic mixtures). Where additional chiral centres are present in compounds of formula (II), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (II) are provided in a natural variant isotopic form. In one embodiment, the compounds of formula (I) are provided in an unnatural variant isotopic form. In one embodiment, the compounds of formula (II) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I) or (II). In one embodiment, the atoms of the compounds of formula (I) or (II) are in an isotopic form which is not radioactive. In one embodiment, one or more atoms of the compounds of formula (I) or (II) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In one embodiment, a compound of formula (II) is provided whereby a single atom of the compound exists in an unnatural variant isotopic form. In another embodiment, a compound of formula (I) is provided whereby two or more atoms exist in an unnatural variant isotopic form. In another embodiment, a compound of formula (II) is provided whereby two or more atoms exist in an unnatural variant isotopic form.

Unnatural isotopic variant forms can generally be prepared by conventional techniques known to those skilled in the art or by processes described herein e.g. processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

Therapeutic Indications

Compounds of formula (I) are of use in therapy, particularly for treating or preventing an inflammatory disease or a disease associated with an undesirable immune response. Compounds of formula (II) are also of use in therapy, particularly for treating or preventing an inflammatory disease or a disease associated with an undesirable immune response. As shown in Biological Example 1 below, example compounds of formula (I) reduced cytokine release more effectively than dimethyl fumarate and in some cases, 2-(2,5-dioxopyrrolidin-1-yl)ethyl methyl fumarate, as demonstrated by lower IC₅₀ values. Compounds of formula (II) reduced cytokine release more effectively than monomethyl fumarate and preferred compounds of formula (II) reduced cytokine release more effectively than dimethyl fumarate and 2-(2,5-dioxopyrrolidin-1-yl)ethyl methyl fumarate, as demonstrated by lower IC₅₀ values. Cytokines are important mediators of inflammation and immune-mediated disease as evidenced by the therapeutic benefit delivered by antibodies targeting them.

Thus, in a first aspect, the present invention provides a compound of formula (I) or a pharmaceutically acceptable salt and/or solvate thereof, as defined herein, for use as a medicament. In a second aspect, the present invention provides a compound of formula (II) or a pharmaceutically acceptable salt and/or solvate thereof, as defined herein, for use as a medicament. In a third aspect the present invention provides a pharmaceutical composition comprising a compound of formula (I) or a pharmaceutically acceptable salt and/or solvate thereof, as defined herein. Such a pharmaceutical composition contains the compound of formula (I) and a pharmaceutically acceptable carrier or excipient. In a fourth aspect, the present invention provides a pharmaceutical composition comprising a compound of formula (II) or a pharmaceutically acceptable salt and/or solvate thereof, as defined herein. Such a pharmaceutical composition contains the compound of formula (II) and a pharmaceutically acceptable carrier or excipient.

In a further aspect, the present invention provides a compound of formula (I) or a pharmaceutically acceptable salt and/or solvate thereof, as defined herein, for use in treating or preventing an inflammatory disease or a disease associated with an undesirable immune response. In a further aspect, the present invention provides the use of a compound of formula (I) or a pharmaceutically acceptable salt and/or solvate thereof, as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof, as defined herein.

In a further aspect, the present invention provides a compound of formula (II) or a pharmaceutically acceptable salt and/or solvate thereof, as defined herein, for use in treating or preventing an inflammatory disease or a disease associated with an undesirable immune response. In a further aspect, the present invention provides the use of a compound of formula (II) or a pharmaceutically acceptable salt and/or solvate thereof, as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (II) or a pharmaceutically acceptable salt and/or solvate thereof, as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof, as defined herein, for use in treating an inflammatory disease or disease associated with an undesirable immune response. In one embodiment of the invention is provided the use of a compound of formula (I) or a pharmaceutically acceptable salt and/or solvate thereof, as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof, as defined herein.

In one embodiment is provided a compound of formula (II) or a pharmaceutically acceptable salt and/or solvate thereof, as defined herein, for use in treating an inflammatory disease or disease associated with an undesirable immune response. In one embodiment of the invention is provided the use of a compound of formula (II) or a pharmaceutically acceptable salt and/or solvate thereof, as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (II) or a pharmaceutically acceptable salt and/or solvate thereof, as defined herein.

In one embodiment is provided a compound of formula (I) or a pharmaceutically acceptable salt and/or solvate thereof, as defined herein, for use in preventing an inflammatory disease or a disease associated with an undesirable immune response. In one embodiment of the invention is provided the use of a compound of formula (I) or a pharmaceutically acceptable salt and/or solvate thereof, as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof, as defined herein.

In one embodiment is provided a compound of formula (II) or a pharmaceutically acceptable salt and/or solvate thereof, as defined herein, for use in preventing an inflammatory disease or a disease associated with an undesirable immune response. In one embodiment of the invention is provided the use of a compound of formula (II) or a pharmaceutically acceptable salt and/or solvate thereof, as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (II) or a pharmaceutically acceptable salt and/or solvate thereof, as defined herein.

In one embodiment is provided a compound of formula (I) or a pharmaceutically acceptable salt and/or solvate thereof, as defined herein, for use in treating or preventing an inflammatory disease. In one embodiment of the invention is provided the use of a compound of formula (I) or a pharmaceutically acceptable salt and/or solvate thereof, as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof, as defined herein.

In one embodiment is provided a compound of formula (II) or a pharmaceutically acceptable salt and/or solvate thereof, as defined herein, for use in treating or preventing an inflammatory disease. In one embodiment of the invention is provided the use of a compound of formula (II) or a pharmaceutically acceptable salt and/or solvate thereof, as defined herein, in the manufacture of a medicament for treating or preventing an inflammatory disease. In one embodiment of the invention is provided a method of treating or preventing an inflammatory disease, which comprises administering a compound of formula (II) or a pharmaceutically acceptable salt and/or solvate thereof, as defined herein.

In one embodiment is provided a compound of formula (I) or a pharmaceutically acceptable salt and/or solvate thereof, as defined herein, for use in treating or preventing a disease associated with an undesirable immune response. In one embodiment of the invention is provided the use of a compound of formula (I) or a pharmaceutically acceptable salt and/or solvate thereof, as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof, as defined herein.

In one embodiment is provided a compound of formula (II) or a pharmaceutically acceptable salt and/or solvate thereof, as defined herein, for use in treating or preventing a disease associated with an undesirable immune response. In one embodiment of the invention is provided the use of a compound of formula (II) or a pharmaceutically acceptable salt and/or solvate thereof, as defined herein, in the manufacture of a medicament for treating or preventing a disease associated with an undesirable immune response. In one embodiment of the invention is provided a method of treating or preventing a disease associated with an undesirable immune response, which comprises administering a compound of formula (II) or a pharmaceutically acceptable salt and/or solvate thereof, as defined herein.

An undesirable immune response will typically be an immune response which gives rise to a pathology, i.e., is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis and chronic obstructive pulmonary disease.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

In one embodiment, the compound of formula (I) exhibits a lower IC₅₀ compared with dimethyl fumarate when tested in a cytokine assay e.g. as described in Biological Example 1. In one embodiment, the compound of formula (I) exhibits a lower IC₅₀ compared with dimethyl fumarate when tested in a cytokine assay e.g. as described in Biological Example 1. In one embodiment, the compound of formula (I) exhibits a lower IC₅₀ compared with 2-(2,5-dioxopyrrolidin-1-yl)ethyl methyl fumarate when tested in a cytokine assay e.g. as described in Biological Example 1. In one embodiment, the compound of formula (I) exhibits a lower IC₅₀ compared with 2-(2,5-dioxopyrrolidin-1-yl)ethyl methyl fumarate when tested in a cytokine assay e.g. as described in Biological Example 1.

In one embodiment, the compound of formula (I) exhibits a lower EC₅₀ compared with dimethyl fumarate when tested in an NRF2 assay e.g. as described in Biological Example 2. In one embodiment, the compound of formula (I) exhibits a higher Eₘₐₓ compared with dimethyl fumarate when tested in an NRF2 assay e.g. as described in Biological Example 2. In one embodiment, the compound of formula (I) exhibits a lower EC₅₀ and/or higher Eₘₐₓ compared with dimethyl fumarate when tested in an NRF2 assay e.g. as described in Biological Example 2. In one embodiment, the compound of formula (I) exhibits a lower EC₅₀ and higher Eₘₐₓ compared with dimethyl fumarate when tested in an NRF2 assay e.g. as described in Biological Example 2.

In one embodiment, the compound of formula (I) exhibits lower intrinsic clearance (CIᵢₙₜ) compared with 2-(2,5-dioxopyrrolidin-1-yl)ethyl methyl fumarate when tested in a hepatocyte stability assay (such as in human hepatocytes), e.g., as described in Biological Example 3. In one embodiment, the compound of formula (I) exhibits a longer half-life (T½) compared with 2-(2,5-dioxopyrrolidin-1-yl)ethyl methyl fumarate when tested in a hepatocyte stability assay (such as in human hepatocytes), e.g. as described in Biological Example 3.

Administration

The compound of formula (I) is usually administered as a pharmaceutical composition. Thus, in one embodiment, is provided a pharmaceutical composition comprising a compound of formula (I) and one or more pharmaceutically acceptable diluents or carriers.

Furthermore, the compound of formula (II) is usually administered as a pharmaceutical composition. Thus, in one embodiment, is provided a pharmaceutical composition comprising a compound of formula (II) and one or more pharmaceutically acceptable diluents or carriers.

Details below regarding pharmaceutical compositions and administration thereof in respect of compounds of formula (I) apply equally to compounds of formula (II).

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen®, specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10 to 60% by weight, of the compound of formula (I), depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 to 1000 mg, more suitably 1.0 to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) and (II) may display one or more of the following desirable properties:
low IC₅₀ values for inhibiting release of cytokines e.g. IL-1β and/or IL-6, from cells;
low EC₅₀ and/or high Eₘₐₓ values for activating the NRF2 pathway;
enhanced efficacy through improved hydrolytic stability of carboxylic acid esters and/or augmented maximum response;
reduced dose and dosing frequency through improved pharmacokinetics;
improved oral systemic bioavailability;
reduced plasma clearance following intravenous dosing;
improved metabolic stability e.g. as demonstrated by improved stability in plasma and/or hepatocytes;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation).

In addition, compounds of formula (II) may be advantageous because their biological activities are not glutathione sensitive.

### Abbreviations

Ac acetyl
Ac₂O acetic anhydride
ADEM acute disseminated encephalomyelitis
AIDP acute inflammatory demyelinating polyneuropathy
ALPS autoimmune lymphoproliferative syndrome
AMAN acute motor axonal neuropathy
AMD age related macular degeneration
AMSAN acute motor and sensory axonal neuropathy
APEX autoimmune polyendocrinopathy-candidiasis-ectodermal dystrophy syndrome
APRIL A PRoliferation-Inducing Ligand
aq. aqueous
ATF3 activating transcription factor 3
ATG anti-thymocyte
BAFF B cell activating factor
BBFO broadband fluorine observe
Bn benzyl
BEH ethylene bridged hybrid
BOC tertiary-butoxycarbonyl
BSA bovine serum albumin
BTK Bruton's tyrosine kinase
CAC citric acid cycle
CANDLE chronic atypical neutrophilic dermatosis with lipodystrophy and elevated temperature
CAPS cryopyrin-associated periodic syndromes
CFC chlorofluorocarbon
CFTR cystic fibrosis transmembrane conductance regulator
CIOP chronic inflammatory demyelinating polyneuropathy
CLE cutaneous lupus erythematosus
CLIPPERS chronic lymphocytic inflammation with pontine perivascular enhancement responsive to steroids
CLL chronic lymphocytic leukaemia
COPD chronic obstructive pulmonary disease
CRMO chronic recurrent multifocal osteomyelitis
CRVO central retinal vein occlusion
CSH charged surface hybrid
DABCO 1,4-diazabicyclo[2.2.2]octane
DAD diode array detector
DBU 1,8-diazabicyclo(5.4.0)undec-7-ene
DCC N,N′-dicyclohexylcarbodiimide
DCM dichloromethane
DIPEA N,N-diisopropylethylamine
DIRA deficiency of interleukin-1 receptor antagonist
DITRA deficiency of the interleukin-36-receptor antagonist
DLBCL diffuse large B cell lymphoma
DMAP 4-dimethylaminopyridine
DMF dimethyl fumarate
DMP Dess-Martin periodinane
DMSO dimethyl sulfoxide
EDC 1-ethyl-3-(3-dimethylaminopropyl)carbodiimide
EDTA ethylenediaminetetraacetic acid
EGPA eosinophilic granulomatosis with polyangiitis
EtOAc ethyl acetate
FBS fetal bovine serum
FCAS familial cold autoinflammatory syndrome
Fmoc 9-fluorenylmethyloxycarbonyl
FMF familial Mediterranean fever
GAPDH glyceraldehyde 3-phosphate dehydrogenase
GPA granulomatosis with polyangiitis
GSH glutathione
GVHD graft versus host disease
HATU 1-[bis(dimethylamino)methylene]-1H-1,2,3-triazolo[4,5-b]pyridinium 3-oxide hexafluorophosphate
HCA2 hydroxycarboxylic acid receptor 2
HCV hepatitis C
HFC hydrofluorocarbon
HIF-1a hypoxia-inducible factor-1α
HIV human immunodeficiency virus
HMDMs human monocyte derived macrophages
HOBt 1-hydroxybenzotriazole
IL interleukin
IPEX immune dysregulation polyendocrinopathy enteropathy X-linked
IRG1 immune-responsive gene 1
IVIG intravenous immune globulin
JAK Janus kinase
KEAP1 kelch-like ECH-associated protein 1
LCMS liquid chromatography-mass spectrometry
LDA lithium diisopropylamide
LPS lipopolysaccharide
MALT mucosa-associated lymphoid tissue
mCPBA meta-chloroperoxybenzoic acid
M-CSF macrophage-colony stimulating factor
MMF monomethyl fumarate
MMN multifocal motor neuropathy
MOG myelin oligodendrocyte glycoprotein
MOM methoxymethyl
MS mass spectrometry
MSD mass selective detector
MTBE methyl tertiary-butyl ether
NLRP12AD NLRP12-associated autoinflammatory disorders
NMM N-methylmorpholine
NMR nuclear magnetic resonance
NOMID neonatal onset multisystem inflammatory disease
NRF2 nuclear factor (erythroid-derived 2)-like 2
NSAIDs non-steroidal anti-inflammatory drugs
O/N overnight
PAH pulmonary arterial hypertension
PANDAS paediatric autoimmune neuropsychiatric disorders associated with streptococcal infections
PANS paediatric acute-onset neuropsychiatric syndrome
PAPA pyogenic arthritis, pyoderma gangrenosum, severe cystic acne
PMB 4-methoxybenzyl
PBMCs primary peripheral blood mononuclear cells
PBS phosphate buffered saline
PDA photodiode array
PDE4 phosphodiesterase-4
PET positron emission topography
PFAPA periodic fever aphthous stomatitis
PLEVA pityriasis lichenoides et varioliformis acuta
PMA phorbol 12-myristate 13-acetate
PSC primary sclerosing cholangitis
PUVA psoralen ultraviolet irradiation
p-TsOH p-toluenesulfonic acid
4OI 4-octyl itaconic acid
RT room temperature
sat. saturated
SDH succinate dehydrogenase
SFC supercritical fluid chromatography
SLE systemic lupus erythematosus
STING stimulator of interferon genes
TBDMS tert-butyldimethylsilyl
TEA triethylamine
TFA trifluoroacetic acid
THP tetrahydropyranyl
TIPS triisopropylsilyl
TLR Toll-like receptor
TMS trimethylsilyl
TNF tumour necrosis factor
TOM tri-iso-propylsilyloxymethyl
Tr trityl
TRAPS tumour necrosis factor receptor-associated periodic fever
Trt trityl, triphenylmethyl
TTP thrombotic thrombocytopenic purpura
UPLC ultra performance liquid chromatography
VEGF vascular endothelial growth factor
VWD variable wavelength detector
wt. weight

### EXAMPLES

Analytical Equipment

NMR spectra were recorded using a Bruker 400 MHz Avance Ill spectrometer fitted with a BBFO 5 mm probe, or a Bruker 500 MHz Avance Ill HD spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software.

UPLC/MS analysis was carried out on a Waters Acquity UPLC system using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH C18 column) or 10 mM Ammonium Bicarbonate (BEH C18 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

LCMS analysis was carried out on an Agilent LCMS system using either a Waters Acquity CSH C18 or BEH C18 column (4.6×30 mm) maintained at a temperature of 40° C. and eluted with a linear acetonitrile gradient appropriate for the lipophilicity of the compound over 4 or 15 minutes at a constant flow rate of 2.5 mL/min. The aqueous portion of the mobile phase was either 0.1% Formic Acid (CSH C18 column) or 10 mM Ammonium Bicarbonate (BEH C18 column). LC-UV chromatograms were recorded using an Agilent VWD or DAD detector at 254 nm. Mass spectra were recorded using an Agilent MSD detector with electrospray ionisation switching between positive and negative ion mode. Sample concentration was adjusted to give adequate UV response.

Commercial Materials

All starting materials disclosed herein are commercially available. Dimethyl fumarate is commercially available, for example from Sigma Aldrich. 2-(2,5-Dioxopyrrolidin-1-yl)ethyl methyl fumarate (diroximel fumarate) is commercially available, for example from Angene. Monomethyl fumarate is commercially available, for example from Sigma Aldrich.

Unless otherwise stated all reactions were stirred. Organic solutions were routinely dried over anhydrous magnesium sulfate. Hydrogenations were performed on a Thales H-cube flow reactor under the conditions stated or under pressure in a gas autoclave (bomb).

### Intermediate 1: 4-methoxybenzyl 3-hydroxy-2,2-dimethylpropanoate

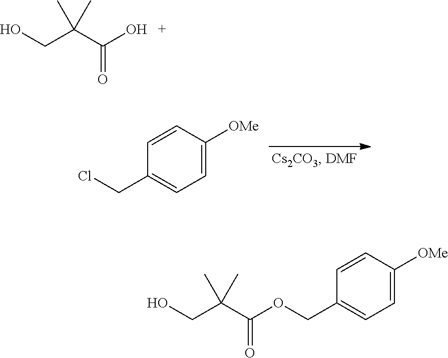

1-(chloromethyl)-4-methoxybenzene (1.0 mL, 7.4 mmol) was added to a mixture of 3-hydroxy-2,2-dimethylpropanoic acid (1.0 g, 8.5 mmol) and cesium carbonate (2.76 g, 8.5 mmol) in dimethylformamide (40 mL). The mixture was stirred at RT for 3 h, then heated to 70° C. for 2 h, then cooled to RT and stirred for 18 h. The mixture was poured onto water (50 mL) and extracted with EtOAc (3×50 mL). The combined organic phases were washed with brine (100 mL), dried (MgSO₄) and concentrated. The crude product was purified by chromatography on silica gel (0-50% EtOAc/isohexane) to afford 4-methoxybenzyl 3-hydroxy-2,2-dimethylpropanoate (1.45 g, 5.78 mmol) as a colourless oil. ¹H NMR (400 MHz, DMSO) δ 7.32-7.27 (m, 2H), 6.97-6.90 (m, 2H), 5.01 (s, 2H), 4.85 (t, J=5.5 Hz, 1H), 3.76 (s, 3H), 3.42 (d, J=5.5 Hz, 2H), 1.08 (s, 6H).

The following compounds were synthesised using the same procedure.

| Int. | | |
|---|---|---|
| Number | Structure | Name |
| 2 | 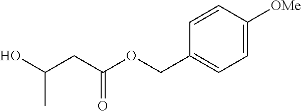 [SMILES: COc1ccc(COC(=O)CC(C)O)cc1] | LCMS m/z 247.1 (M + Na)⁺ (ES⁺). ¹H NMR (400 MHz, DMSO) δ 7.34-7.25 (m, 2H), 6.97-6.86 (m, 2H), 5.00 (s, 2H), 4.72 (d, J = 5.1 Hz, 1H), 4.05-3.95 (m, 1H), 3.75 (s, 3H), 2.37 (d, J = 6.6 Hz, 2H), 1.08 (d, J = 6.2 Hz, 3H) |
| | 4-methoxybenzyl 3- | |
| | hydroxybutanoate | |

### Intermediate 3: (E)-4-oxo-4-(1-(4-(trifluoromethyl)phenyl)cyclobutoxy)but-2-enoic acid

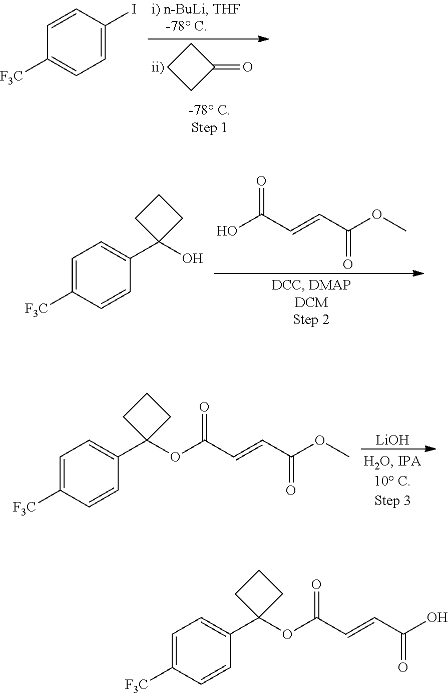

### Step 1

To a solution of 1-bromo-4-(trifluoromethyl)benzene (22.3 g, 99.5 mmol) in THF (180 mL) at −78° C. was added n-BuLi solution in hexane (2.5 M, 43.7 mL, 109.2 mmol) and the mixture was stirred at −78° C. for 1 h. Cyclobutanone (7.6 g, 109.2 mmol) was added, and the mixture was stirred at −78° C. for 5 h, then quenched with saturated aqueous NH₄Cl solution (200 mL). The phases were separated and the aqueous layer was extracted with MTBE (2×80 mL). The combined organic layers were washed with brine, dried over Na₂SO₄, filtered and concentrated under reduced pressure at 30° C., and the residue was purified by flash column chromatography (120 g silica, 0-14% MTBE/petroleum ether) to give 1-(4-(trifluoromethyl)phenyl)cyclobutan-1-ol (16.5 g, 76.3 mmol, 77%) as a yellow oil. ¹H NMR (400 MHz, CDCl₃) δ: 7.61 (s, 4H), 2.59-2.48 (m, 2H), 2.43-2.32 (m, 2H), 2.12-1.98 (m, 1H), 1.81-1.66 (m, 1H). One exchangeable proton not observed.

### Step 2

A mixture of 1-(4-(trifluoromethyl)phenyl)cyclobutan-1-ol (300 mg, 1.39 mmol), (E)-4-methoxy-4-oxobut-2-enoic acid (181 mg, 1.39 mmol), DCC (430 mg, 2.09 mmol) and DMAP (17 mg, 0.14 mmol) in DCM (3 mL) was stirred at room temperature for 30 min. The mixture was filtered, and the filtrate was concentrated under reduced pressure at 35° C. The residue was purified by flash column chromatography (12 g silica, 0-10% MTBE/petroleum ether) to give methyl (1-(4-(trifluoromethyl)phenyl)cyclobutyl) fumarate (360 mg, 1.10 mmol, 79%) as a colorless oil. ¹H NMR (400 MHz, CDCl₃) δ: 7.65-7.55 (m, 4H), 6.87-6.76 (m, 2H), 3.80 (s, 3H), 2.79-2.59 (m, 4H), 2.13-1.97 (m, 1H), 1.87-1.71 (m, 1H).

### Step 3

To a solution of methyl (1-(4-(trifluoromethyl)phenyl)cyclobutyl) fumarate (360 mg, 1.10 mmol) in IPA (3 mL) was added aqueous LiOH solution (2 M, 0.6 mL, 1.20 mmol), and the reaction mixture was stirred at 10° C. for 20 min. The reaction mixture was acidified with dilute aqueous HCl (0.5 M) to pH=3, and extracted with EtOAc (2×5 mL). The combined organic layers were washed with brine, dried over Na₂SO₄, filtered and concentrated under reduced pressure at 30° C. The residue was purified by preparative HPLC (Column: Waters X-Bridge C18 OBD 10 μm 19×250 mm; Flow Rate: 20 mL/min; solvent system: MeCN/(0.2% formic acid/water); gradient: 65-95% MeCN; collection wavelength: 214 nm). The collected fractions were concentrated under reduced pressure at 30° C. to remove MeCN, and the residue was lyophilized to give (E)-4-oxo-4-(1-(4-(trifluoromethyl)phenyl)cyclobutoxy)but-2-enoic acid (110 mg, 0.35 mmol, 32%) as an off-white solid. LCMS m/z 336.8 (M+Na)⁺ (ES⁺). ¹H NMR (400 MHz, DMSO-d6) δ: 13.26 (br, 1H), 7.75 (d, J=8.5 Hz, 2H), 7.70 (d, J=8.4 Hz, 2H), 6.74-6.62 (m, 2H), 2.68-2.59 (m, 4H), 2.05-1.92 (m, 1H), 1.85-1.69 (m, 1H).

### Intermediate 4: (E)-4-(1-methylcyclobutoxy)-4-oxobut-2-enoic acid

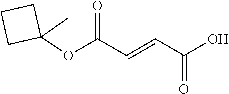

Fumaroyl dichloride (0.071 mL, 0.654 mmol) was dissolved in DCM (2 mL) and treated with 1-methylcyclobutanol (0.113 g, 1.308 mmol) and TEA (0.310 ml, 2.223 mmol). The reaction mixture was stirred for 3 hours at room temperature, then it was diluted with water. The organic layer was collected and dried (phase separator), then the solvent was removed under reduced pressure. The crude product was purified by chromatography on silica gel (12 g cartridge, 0-10% MeOH/DCM), yielding only (E)-4-(1-methylcyclobutoxy)-4-oxobut-2-enoic acid (60 mg, 0.319 mmol, 48.8% yield) as a yellow oil. ¹H NMR (500 MHz, DMSO-d₆) δ 13.51 (s, 1H), 6.64 (d, J=7.3 Hz, 2H), 2.36-2.24 (m, 2H), 2.11 (ddq, J=12.1, 8.2, 2.4 Hz, 2H), 1.83-1.73 (m, 1H), 1.72-1.62 (m, 1H), 1.53 (s, 3H).

### Intermediate 5: Octyl Fumarate

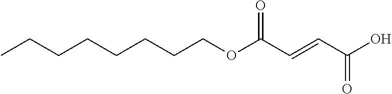 [SMILES: CCCCCCCCOC(=O)/C=C/C(=O)O]

This compound is commercially available and may be purchased, for example, from Aurora Fine Chemicals Ltd.

### Intermediate 6: (E)-4-(cyclooctyloxy)-4-oxobut-2-enoic acid

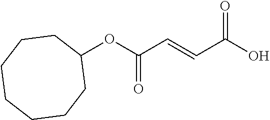 [SMILES: O=C(O)/C=C/C(=O)OC1CCCCCCC1]

The synthesis of Intermediate 6 is described in Example 3.

### Intermediate 7: (E)-4-oxo-4-(spiro[3.3]heptan-2-yloxy)but-2-enoic acid

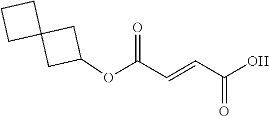 [SMILES: O=C(O)/C=C/C(=O)OC1CC2(CCC2)C1]

The synthesis of Intermediate 7 is described in Example 9.

### Intermediate 8: (E)-4-(cycloheptyloxy)-4-oxobut-2-enoic acid

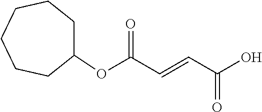 [SMILES: O=C(O)/C=C/C(=O)OC1CCCCCC1]

The synthesis of Intermediate 8 is described in Example 10.

### Intermediate 9: (E)-4-oxo-4-(1-(5-(trifluoromethyl)pyridin-2-yl)cyclobutoxy)but-2-enoic acid

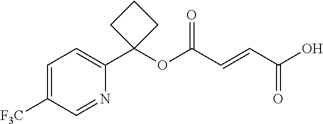 [SMILES: O=C(O)/C=C/C(=O)OC1(c2ccc(C(F)(F)F)cn2)CCC1]

Prepared from of 2-bromo-5-(trifluoromethyl)pyridine and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 8.81 (s, 1H), 7.99 (dd, J=8.3, 2.3 Hz, 1H), 7.72 (d, J=8.3 Hz, 1H), 4.73 (s, 1H), 2.61-2.49 (m, 4H), 2.17-2.07 (m, 1H), 1.98-1.86 (m, 1H).

### Step 2

LCMS m/z 330.0 (M+H)⁺ (ES⁺).

### Step 3

LCMS m/z 316.0 (M+H)⁺ (ES⁺). ¹H NMR (400 MHz, DMSO-d6) δ: 13.27 (br s, 1H), 9.00 (s, 1H), 8.20 (dd, J=8.4, 1.6 Hz, 1H), 7.66 (d, J=8.4 Hz, 1H), 6.75 (s, 2H), 2.77-2.70 (m, 2H), 2.64-2.54 (m, 2H), 2.03-1.89 (m, 2H).

### Intermediate 10: (E)-4-oxo-4-(1-(3-(trifluoromethyl)phenyl)cyclobutoxy)but-2-enoic acid

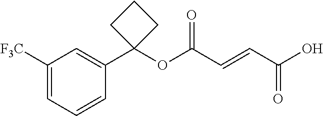 [SMILES: O=C(O)/C=C/C(=O)OC1(c2cccc(C(F)(F)F)c2)CCC1]

Prepared from 1-bromo-2-(trifluoromethyl)benzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 7.77 (s, 1H), 7.70 (d, J=8.0 Hz, 1H), 7.56-7.48 (m, 2H), 3.21-2.54 (m, 2H), 2.44-2.37 (m, 2H), 2.13-2.04 (m, 1H), 1.79-1.72 (m, 1H).

### Step 2

LCMS m/z 351.0 (M+Na)⁺ (ES⁺).

### Step 3

LCMS m/z 336.9 (M+Na)+(ES+). ¹H NMR (400 MHz, DMSO-d6) δ: 13.17 (br s, 1H), 7.81 (d, J=7.2 Hz, 1H), 7.74 (s, 1H), 7.71-7.60 (m, 2H), 6.73-6.62 (m, 2H,), 2.69-2.59 (m, 4H), 2.04-1.92 (m, 1H), 1.80-1.66 (m, 1H).

### Intermediate 11: (E)-4-oxo-4-(1-(2-(trifluoromethyl)phenyl)cyclobutoxy)but-2-enoic acid

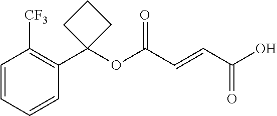 [SMILES: O=C(O)/C=C/C(=O)OC1(c2ccccc2C(F)(F)F)CCC1]

Prepared from 1-bromo-2-(trifluoromethyl)benzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl3) δ: 7.68-7.52 (m, 2H), 7.45-7.37 (m, 2H), 2.68-2.61 (m, 2H), 2.46-2.42 (m, 2H), 2.33-2.28 (m, 2H).

### Step 2

LCMS m/z 351.0 (M+Na)⁺ (ES+).

### Step 3

LCMS m/z 337.0 (M+Na)+(ES+). ¹H NMR (400 MHz, DMSO-d6) δ 7.91 (d, J=8.0 Hz, 1H), 7.77 (d, J=8.0 Hz, 1H), 7.72 (t, J=7.2 Hz, 1H), 7.55 (t, J=7.6 Hz, 1H), 6.63-6.55 (m, 2H), 2.79-2.75 (m, 2H), 2.68-2.61 (m, 2H), 1.97-1.91 (m, 1H), 1.74-1.67 (m, 1H).

### Intermediate 12: (E)-4-(1-(4-bromophenyl)cyclobutoxy)-4-oxobut-2-enoic acid

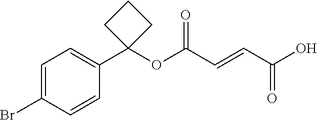 [SMILES: O=C(O)/C=C/C(=O)OC1(c2ccc(Br)cc2)CCC1]

Prepared from 1,4-dibromobenzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl3) δ: 7.48 (d, J=7.8 Hz, 2H), 7.36 (d, J=8.2 Hz, 2H), 2.53-2.46 (m, 2H), 2.37-2.30 (m, 2H), 2.06-1.97 (m, 1H), 1.71-1.64 (m, 1H).

### Step 2

LCMS m/z 361.0 (M+Na)⁺ (ES+).

### Step 3

LCMS m/z 346.9 (M+Na)+. (ES+). ¹H NMR (400 MHz, DMSO-d6) δ: 13.21 (br s, 1H), 7.59-7.54 (m, 2H), 7.45-7.42 (m, 2H), 6.71-6.61 (m, 2H), 2.65-2.55 (m, 4H), 2.00-1.89 (m, 1H), 1.77-1.63 (m, 1H).

### Intermediate 13: (E)-4-(1-(4-chlorophenyl)cyclobutoxy)-4-oxobut-2-enoic acid

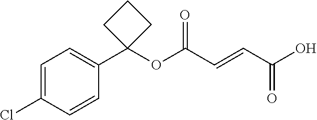 [SMILES: O=C(O)/C=C/C(=O)OC1(c2ccc(Cl)cc2)CCC1]

Prepared from of 1-bromo-4-chlorobenzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl3) δ: 7.46-7.42 (m, 2H), 7.35-7.32 (m, 2H), 2.56-2.45 (m, 2H), 2.40-2.33 (m, 2H), 2.06-1.99 (m, 1H), 1.73-1.66 (m, 1H).

### Step 2

LCMS m/z 316.8 (M+Na)⁺ (ES⁺).

### Step 3

LCMS m/z 302.9 (M+Na)+. (ES+). ¹H NMR (400 MHz, DMSO-d6) δ: 13.21 (br s, 1H), 7.51 (dd, J=6.4 Hz, 2.0 Hz, 2H), 7.43 (dd, J=6.8 Hz, 2.0 Hz, 2H), 6.70-6.62 (m, 2H), 2.62-2.59 (m, 4H), 2.00-1.92 (m, 1H), 1.74-1.69 (m, 1H).

### Intermediate 14: (E)-4-(1-(3,5-dichlorophenyl)cyclobutoxy)-4-oxobut-2-enoic acid

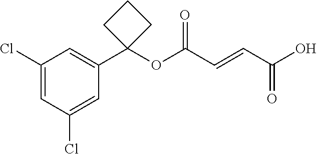 [SMILES: O=C(O)/C=C/C(=O)OC1(c2cc(Cl)cc(Cl)c2)CCC1]

Prepared from 1,3-dichloro-5-iodobenzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl3) δ 7.38 (d, J=2.0 Hz, 2H), 7.27 (t, J=2.0 Hz, 1H), 2.54-2.48 (m, 2H), 2.40-2.32 (m, 2H), 2.12-2.06 (m, 1H), 1.81-1.70 (m, 1H).

### Step 2

LCMS m/z 351.0 (M+Na)+(ES+).

### Step 3

¹H NMR (400 MHz, DMSO-d6) δ: 13.26 (br s, 1H), 7.56 (t, J=2.0 Hz, 1H), 7.50 (d, J=1.2 Hz, 2H), 6.74-6.64 (m, 2H), 2.68-2.54 (m, 4H), 1.98-1.92 (m, 1H), 1.77-1.70 (m, 1H).

### Intermediate 15: (E)-4-oxo-4-(1-(6-(trifluoromethyl)pyridin-3-yl)cyclobutoxy)but-2-enoic acid

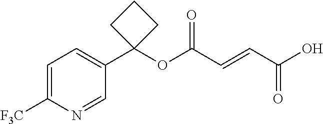 [SMILES: O=C(O)/C=C/C(=O)OC1(c2ccc(C(F)(F)F)nc2)CCC1]

Prepared from 5-bromo-2-(trifluoromethyl)pyridine and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 8.90 (d, J=2.0 Hz, 1H), 8.01 (dd, J=8.0 Hz, 1.6 Hz, 1H), 7.69 (d, J=8.0 Hz, 1H), 2.62-2.55 (m, 2H), 2.49-2.41 (m, 2H), 2.18-2.09 (m, 1H), 1.88-1.76 (m, 1H).

### Step 2

LCMS m/z 330.2 (M+H)⁺ (ES+).

### Step 3

LCMS m/z 316.0 (M+H)⁺. ¹H NMR (400 MHz, DMSO-d6) δ: 13.25 (br s, 1H), 8.92 (d, J=2.0 Hz, 1H), 7.43 (dd, J=8.4 Hz, 1.6 Hz, 1H), 7.92 (d, J=8.4 Hz, 1H), 6.74-6.65 (m, 2H), 2.72-2.65 (m, 4H), 2.03-1.98 (m, 1H), 1.84-1.76 (m, 1H).

### Intermediate 16: (E)-4-(1-(3-fluoro-4-(trifluoromethyl)phenyl)cyclobutoxy)-4-oxobut-2-enoic acid

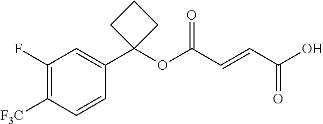 [SMILES: O=C(O)/C=C/C(=O)OC1(c2ccc(C(F)(F)F)c(F)c2)CCC1]

Prepared from 4-bromo-2-fluoro-1-(trifluoromethyl)benzene and cyclobutanone using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 7.60 (t, J=8.0 Hz, 1H), 7.40-7.35 (m, 2H), 2.57-2.50 (m, 2H), 2.44-2.37 (m, 2H), 2.11-2.09 (m, 1H), 1.80-1.77 (m, 1H).

### Step 3

¹H NMR (400 MHz, DMSO-d6) δ: 13.24 (br s, 1H), 7.79 (t, J=8.0 Hz, 1H), 7.63 (d, J=12.0 Hz, 1H), 7.50 (d, J=8.0 Hz, 1H), 6.75-6.66 (m, 2H), 2.67-2.61 (m, 4H), 2.01-1.95 (m, 1H), 1.83-1.75 (m, 1H).

### Intermediate 17: (E)-4-oxo-4-((3-(4-(trifluoromethyl)phenyl)thietan-3-yl)oxy)but-2-enoic acid

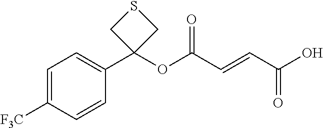 [SMILES: O=C(O)/C=C/C(=O)OC1(c2ccc(C(F)(F)F)cc2)CSC1]

Prepared from 1-iodo-4-(trifluoromethyl)benzene and thietan-3-one using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 7.84 (d, J=8.0 Hz, 2H), 7.67 (d, J=8.4 Hz, 2H), 3.60 (s, 4H), 2.84 (s, 1H).

### Step 2

LCMS m/z 368.9 (M+Na)⁺ (ES⁺).

### Step 3

¹H NMR (400 MHz, DMSO-d6) δ: 13.30 (br s, 1H), 7.89 (d, J=8.4 Hz, 2H), 7.81 (d, J=8.4 Hz, 2H), 6.76 (d, J=15.8 Hz, 1H), 6.66 (d, J=15.8 Hz, 1H), 4.05-3.99 (m, 2H), 3.59-3.53 (m, 2H).

### Intermediate 18: (E)-4-oxo-4-((3-(4-(trifluoromethyl)phenyl)oxetan-3-yl)oxy)but-2-enoic acid

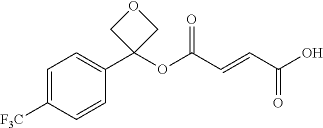 [SMILES: O=C(O)/C=C/C(=O)OC1(c2ccc(C(F)(F)F)cc2)COC1]

Prepared from 1-bromo-4-(trifluoromethyl)benzene and oxetan-3-one using a similar procedure to (E)-4-oxo-4-(1-(4-(trifluoromethyl)phenyl)cyclobutoxy)but-2-enoic acid.

### Step 1

¹H NMR (400 MHz, CDCl₃) δ: 7.78 (d, J=8.4 Hz, 2H), 7.69 (d, J=8.4 Hz, 2H), 4.95 (d, J=7.6 Hz, 2H), 4.89 (d, J=7.6 Hz, 2H).

### Step 2

LCMS m/z 331.0 (M+H)⁺ (ES⁺).

### Step 3

LCMS m/z 316.9 (M+H)⁺. ¹H NMR (400 MHz, DMSO-d6) δ: 13.35 (br s, 1H), 7.81 (d, J=8.4 Hz, 2H), 7.76 (d, J=8.4 Hz, 2H), 6.87-6.74 (m, 2H), 5.04 (d, J=8.0 Hz, 2H), 4.89 (d, J=8.0 Hz, 2H).

### Intermediate 19: (S,E)-4-oxo-4-(1-(4-(trifluoromethyl)phenyl)ethoxy)but-2-enoic acid

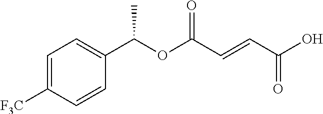 [SMILES: C[C@H](OC(=O)/C=C/C(=O)O)c1ccc(C(F)(F)F)cc1]

Prepared from (S)-1-(4-(trifluoromethyl)phenyl)ethanol using a similar procedure to (E)-4-oxo-4-(1-(4-(trifluoromethyl)phenyl)cyclobutoxy)but-2-enoic acid (Step 2 and Step 3 only).

### Step 3

¹H NMR (400 MHz, DMSO-d6) δ: 12.27 (br, 1H), 7.75 (d, J=8.4 Hz, 2H), 7.65 (d, J=8.4 Hz, 2H), 6.80-6.72 (m, 2H), 5.99 (q, J=6.4 Hz, 1H), 1.55 (d, J=6.4 Hz, 3H).

### Intermediate 20: (R,E)-4-oxo-4-(1-(4-(trifluoromethyl)phenyl)ethoxy)but-2-enoic acid

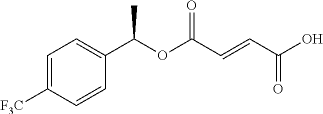 [SMILES: C[C@@H](OC(=O)/C=C/C(=O)O)c1ccc(C(F)(F)F)cc1]

Prepared from (R)-1-(4-(trifluoromethyl)phenyl)ethanol using a similar procedure to (E)-4-oxo-4-(1-(4-(trifluoromethyl)phenyl)cyclobutoxy)but-2-enoic acid (Step 2 and Step 3 only).

### Step 3

¹H NMR (400 MHz, DMSO-d6) δ: 13.27 (br s, 1H), 7.75 (d, J=8.0 Hz, 2H), 7.65 (d, J=8.4 Hz, 2H), 6.80-6.71 (m, 2H), 5.99 (q, J=6.4 Hz, 1H), 1.55 (d, J=6.8 Hz, 3H).

### Intermediate 21: (E)-4-oxo-4-((2-(4-(trifluoromethyl)phenyl)propan-2-yl)oxy)but-2-enoic acid

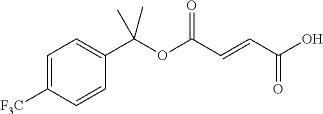 [SMILES: CC(C)(OC(=O)/C=C/C(=O)O)c1ccc(C(F)(F)F)cc1]

Prepared from 2-(4-(trifluoromethyl)phenyl)propan-2-ol using a similar procedure to (E)-4-oxo-4-(1-(4-(trifluoromethyl)phenyl)cyclobutoxy)but-2-enoic acid (Step 2 and Step 3 only).

### Step 2

LCMS m/z 339.0 (M+Na)⁺ (ES+).

### Step 3

¹H NMR (400 MHz, DMSO-d6) δ: 13.26 (br s, 1H), 7.71 (d, J=8.0 Hz, 2H), 7.60 (d, J=8.4 Hz, 2H), 6.69 (s, 2H), 1.77 (s, 6H).

### Intermediate 22: (E)-4-((9H-fluoren-9-yl)methoxy)-4-oxobut-2-enoic acid

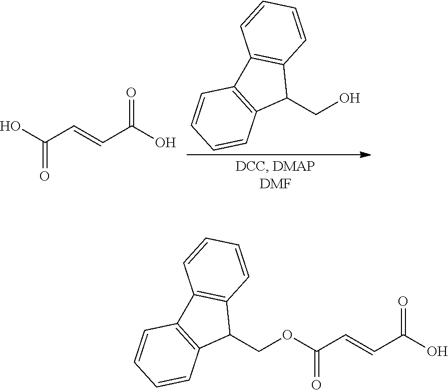

To the solution of fumaric acid (10.0 g, 86.1 mmol), (9H-fluoren-9-yl)methanol (5.6 g, 28.7 mmol) and DMAP (350 mg, 2.9 mmol) in DCM (150 mL) was added DCC (8.9 g, 43.1 mmol) at 0° C., and the mixture was stirred at room temperature for 2 h. The reaction mixture was filtered and the filtrate was concentrated under reduced pressure. The residue was purified by flash column chromatography on silica (0-30% tert-butyl methyl ether/petroleum ether) to give a mixture of (9H-fluoren-9-yl)methanol and (E)-4-((9H-fluoren-9-yl)methoxy)-4-oxobut-2-enoic acid. The mixture was dissolved with EtOAc (50 mL) and the solution extracted with saturated potassium carbonate (100 mL). The aqueous layer was separated and washed with EtOAc (2×20 mL), acidified with 2N HCl until pH 4-5, and extracted with EtOAc (3×30 mL). The EtOAc layer was washed with brine, dried over Na₂SO₄, filtered and concentrated under reduced pressure to give (E)-4-((9H-fluoren-9-yl)methoxy)-4-oxobut-2-enoic acid (7.50 g, 89%) as a white solid. LCMS m/z 317.0 (M+Na)⁺ (ES+). ¹H NMR (400 MHz, CDCl₃) δ: 13.29 (br s, 1H), 7.91 (d, J=7.6 Hz, 2H), 7.68 (d, J=7.2 Hz, 2H), 7.43 (t, J=7.6 Hz, 2H), 7.35 (t, J=7.6 Hz, 2H), 6.68 (q, J=15.6 Hz, 2H), 4.54 (d, J=6.8 Hz, 2H), 4.35 (t, J=6.4 Hz, 1H).

### Intermediate 23: (E)-4-(1-(5-bromopyridin-2-yl)cyclobutoxy)-4-oxobut-2-enoic acid

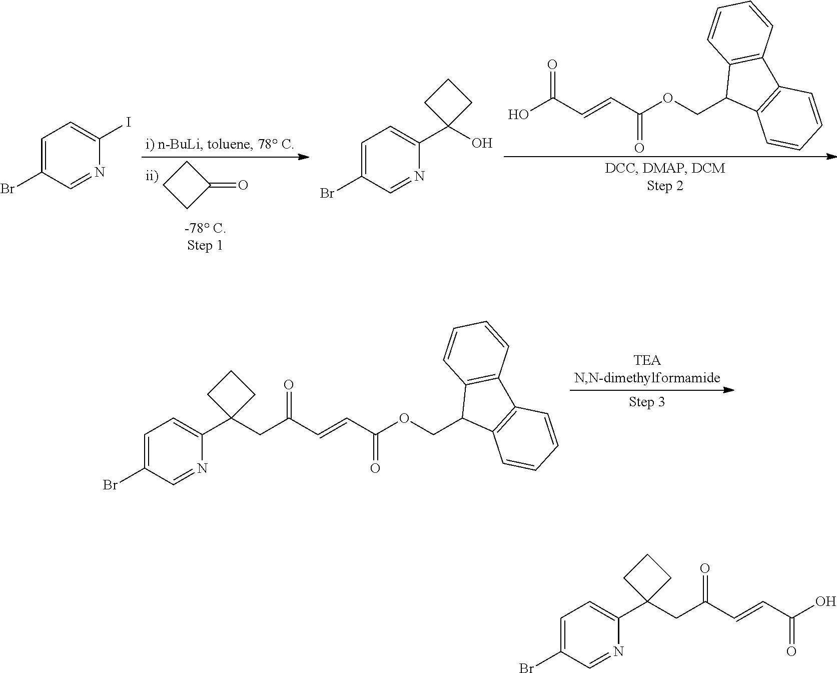 [SMILES: Brc1ccc(I)nc1 ; O=C1CCC1 ; OC1(c2ccc(Br)cn2)CCC1 ; O=C(O)/C=C/C(=O)OCC1c2ccccc2-c2ccccc21 ; O=C(/C=C/C(=O)OCC1c2ccccc2-c2ccccc21)CC1(c2ccc(Br)cn2)CCC1 ; O=C(O)/C=C/C(=O)CC1(c2ccc(Br)cn2)CCC1]

### Step 1

To the solution of 5-bromo-2-iodopyridine (5.0 g, 17.67 mmol) in toluene (50 mL) was added n-BuLi (7.07 mL, 17.67 mmol, 2.5 M in n-hexane) at −78° C.; and the mixture was stirred for 1 h at this temperature. Cyclobutanone (1.24 g, 17.67 mmol) and added and the mixture was stirred at −78° C. for further 2 h. The reaction mixture was quenched with saturated aqueous ammonium chloride (50 mL), the organic layer was separated and the aqueous layer was extracted with MTBE (2×50 mL). The combined organic layers were washed by brine, dried over Na₂SO₄, filtered and concentrated under reduced pressure. The residue was purified by flash column chromatography on silica (0-15% tert-butyl methyl ether/petroleum ether) to give 1-(5-bromopyridin-2-yl)cyclobutan-1-ol (3.0 g, 75% yield) as a light yellow oil. ¹H NMR (400 MHz, CDCl₃) δ: 8.58 (d, J=2.2 Hz, 1H), 7.86 (dd, J=8.4, 2.4 Hz, 1H), 7.49 (d, J=8.4 Hz, 1H), 4.67 (s, 1H), 2.57-2.45 (m, 4H), 2.12-2.04 (m, 1H), 1.92-1.82 (m, 1H).

### Step 2

A mixture of 1-(5-bromopyridin-2-yl)cyclobutan-1-ol (300 mg, 1.32 mmol), (E)-4-((9H-fluoren-9-yl)methoxy)-4-oxobut-2-enoic acid (Intermediate 22, 388 mg, 1.32 mmol), DCC (407 mg, 1.98 mmol) and DMAP (16 mg, 0.13 mmol) in DCM (4 mL) was stirred at room temperature for 3 h. The reaction mixture was filtered and the filtrate was concentrated under reduced pressure. The residue was purified by flash column chromatography on silica (0-18% tert-butyl methyl ether/petroleum ether) to give (9H-fluoren-9-yl)methyl (1-(5-bromopyridin-2-yl)cyclobutyl) fumarate (400 mg, 60% yield) as a colorless oil. LCMS m/z 504.0 (M+H)⁺ (ES+).

### Step 3

A solution of (9H-fluoren-9-yl)methyl (1-(5-bromopyridin-2-yl)cyclobutyl) fumarate (400 mg, 0.79 mmol) in dimethylformamide (2 mL) and triethylamine (0.4 mL) was stirred at room temperature for 2 h. The reaction mixture was acidified with 0.5 N HCl until pH=6 and extracted with EtOAc (2×3 mL). The EtOAc layer was washed by brine, dried over Na₂SO₄, filtered and concentrated under reduced pressure. The residue was purified by prep-HPLC (Column: Waters X-Bridge C18 OBD 10 μm 19×250 mm; Flow Rate: 20 mL/min; solvent system: MeCN/[0.2% formic acid/water] gradient: 55-95% MeCN; collection wavelength: 214 nm). The fractions were concentrated under reduced pressure to remove MeCN, and lyophilized to give (E)-4-(1-(5-bromopyridin-2-yl)cyclobutoxy)-4-oxobut-2-enoic acid (135.8 mg, 52% yield) as white solid. LCMS m/z 326.0 (M+H)⁺ (ES+). ¹H NMR (400 MHz, DMSO-d6) δ: 13.32 (br s, 1H), 8.71 (d, J=1.6 Hz, 1H), 8.03 (dd, J=8.4, 2.4 Hz, 1H), 7.41 (dd, J=8.4, 0.4 Hz, 1H), 6.76-6.67 (m, 2H), 2.73-2.66 (m, 2H), 2.59-2.51 (m, 2H), 2.00-1.92 (m, 1H), 1.90-1.83 (m, 1H).

### Intermediate 24: (E)-4-(1-(5-chloropyridin-2-yl)cyclobutoxy)-4-oxobut-2-enoic acid

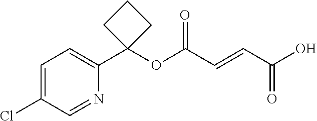 [SMILES: O=C(O)/C=C/C(=O)OC1(c2ccc(Cl)cn2)CCC1]

Prepared from 2-bromo-5-chloropyridine and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 8.48 (d, J=2.4 Hz, 1H), 7.72 (dd, J=8.4, 2.4 Hz, 1H), 7.54 (d, J=8.4 Hz, 1H), 4.67 (s, 1H), 2.57-2.44 (m, 4H), 2.13-2.02 (m, 1H), 2.10.2.05 (m, 1H), 1.91-1.80 (m, 1H).

### Step 2

LCMS m/z 460.0 (M+H)⁺ (ES+).

### Step 3

LCMS m/z 282.1 (M+H)⁺. ¹H NMR (400 MHz, DMSO-d6) δ: 13.31 (br, 1H), 8.63 (d, J=2.0 Hz, 1H), 7.91 (dd, J=8.8, 2.8 Hz, 1H), 7.47 (d, J=8.8, Hz, 1H), 6.76-6.67 (m, 2H), 2.73-2.67 (m, 2H), 2.60-2.51 (m, 2H), 2.01-1.92 (m, 1H), 1.90-1.83 (m, 1H).

### Intermediate 25: (E)-4-(1-(3,5-dichloro-4-fluorophenyl)cyclobutoxy)-4-oxobut-2-enoic acid

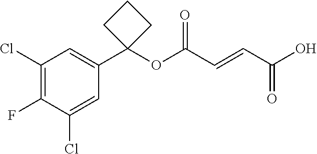 [SMILES: O=C(O)/C=C/C(=O)OC1(c2cc(Cl)c(F)c(Cl)c2)CCC1]

Prepared from 5-bromo-1,3-dichloro-2-fluorobenzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.44 (d, J=6.4 Hz, 2H), 2.55-2.44 (m, 2H), 2.41-2.30 (m, 2H), 2.11-2.04 (m, 1H), 1.80-1.66 (m, 1H).

### Step 2

LCMS m/z 532.8 (M+Na)⁺ (ES+).

### Step 3

¹H NMR (400 MHz, DMSO-d6) δ: 13.25 (br s, 1H), 7.69 (d, J=6.4 Hz, 2H), 6.75-6.62 (m, 2H), 2.70-2.50 (m, 4H), 2.00-1.91 (m, 1H), 1.77-1.68 (m, 1H).

### Intermediate 26: (E)-4-(1-(3-chloro-4-(trifluoromethyl)phenyl)cyclobutoxy)-4-oxobut-2-enoic acid

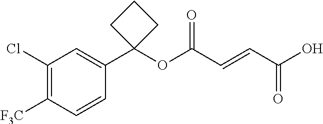 [SMILES: O=C(O)/C=C/C(=O)OC1(c2ccc(C(F)(F)F)c(Cl)c2)CCC1]

Prepared from 4-bromo-2-chloro-1-(trifluoromethyl)benzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.67 (t, J=7.6 Hz, 2H), 7.49 (d, J=8.4 Hz, 1H), 2.57-2.50 (m, 2H), 2.43-2.35 (m, 2H), 2.14-2.04 (m, 1H), 1.83-1.74 (m, 1H).

### Step 2

LCMS m/z 559.0 (M+Na)⁺ (ES+).

### Step 3

¹H NMR (400 MHz, DMSO-d6) δ: 13.29 (br s, 1H), 7.87 (d, J=8.0 Hz, 1H), 7.78 (s, 1H), 7.65 (d, J=8.0 Hz, 1H), 6.78-6.64 (m, 2H), 2.70-2.58 (m, 4H), 2.08-1.94 (m, 1H), 1.84-1.728 (m, 1H).

### Intermediate 27: (E)-4-(1-(4-cyanophenyl)cyclobutoxy)-4-oxobut-2-enoic acid

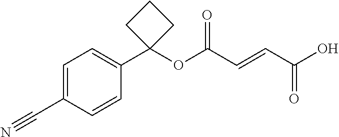 [SMILES: N#Cc1ccc(C2(OC(=O)/C=C/C(=O)O)CCC2)cc1]

Prepared from 4-iodobenzonitrile and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.66-7.61 (m, 4H), 2.56-2.50 (m, 2H), 2.44-2.36 (m, 2H), 2.13-2.05 (m, 1H), 1.85-1.71 (m, 1H).

### Step 2

LCMS m/z 472.0 (M+Na)⁺ (ES+).

### Step 3

LCMS m/z 294.1 (M+Na)⁺ (ES+). ¹H NMR (400 MHz, DMSO-d6) δ: 13.26 (br s, 1H), 7.85 (d, J=8.8 Hz, 2H), 7.71-7.63 (m, 2H), 2.64-2.60 (m, 4H), 2.01-1.95 (m, 1H), 1.80-1.73 (m, 1H)

### Intermediate 28: (E)-4-oxo-4-(1-(3,4,5-trifluorophenyl)cyclobutoxy)but-2-enoic acid

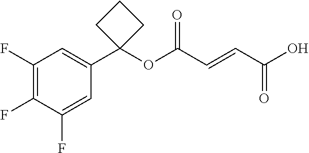 [SMILES: O=C(O)/C=C/C(=O)OC1(c2cc(F)c(F)c(F)c2)CCC1]

Prepared from 5-bromo-1,2,3-trifluorobenzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.14-7.04 (m, 1H), 6.97-6.90 (m, 1H), 2.66-2.59 (m, 2H), 2.46-2.31 (m, 2H), 2.21-2.10 (m, 1H), 1.82-1.68 (m, 1H).

### Step 2

LCMS m/z 501.0 (M+Na)⁺ (ES+).

### Step 3

NMR (400 MHz, DMSO-d6) δ: 13.24 (br s, 1H), 7.52-7.46 (m, 1H), 7.36-7.29 (m, 2H), 6.69-6.55 (m, 2H), 2.79-2.73 (m, 2H), 2.67-2.59 (m, 2H), 2.02-1.95 (m, 1H), 1.71-1.64 (m, 1H)

### Intermediate 29: (E)-4-(1-(3,5-difluoro-4-(trifluoromethyl)phenyl)cyclobutoxy)-4-oxobut-2-enoic acid

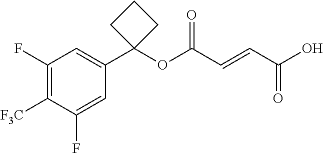 [SMILES: O=C(O)/C=C/C(=O)OC1(c2cc(F)c(C(F)(F)F)c(F)c2)CCC1]

Prepared from 5-bromo-1,3-difluoro-2-(trifluoromethyl)benzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.17 (d, J=11.2 Hz, 2H), 2.53-2.46 (m, 2H), 2.43-2.36 (m, 2H), 2.15-2.07 (m, 1H), 1.84-1.77 (m, 1H)

### Step 2

LCMS m/z 550.8 (M+Na)⁺ (ES+).

### Step 3

NMR (400 MHz, DMSO-d6) δ: 13.26 (brs, 1H), 7.50 (d, J=11.2 Hz, 2H), 6.78-6.64 (m, 2H), 2.68-2.56 (m, 4H), 2.02-1.93 (m, 1H), 1.87-1.76 (m, 1H).

### Intermediate 30: (E)-4-oxo-4-(1-(4-(trifluoromethoxy)phenyl)cyclobutoxy)but-2-enoic acid

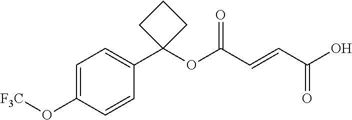 [SMILES: O=C(O)/C=C/C(=O)OC1(c2ccc(OC(F)(F)F)cc2)CCC1]

Prepared from 1-bromo-4-(trifluoromethoxy)benzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.52 (d, J=7 Hz, 2H), 7.20 (d, J=4 Hz, 2H), 2.57-2.50 (m, 2H), 2.40-2.31 (m, 2H), 2.07-1.99 (m, 1H), 1.74-1.66 (m, 1H).

### Step 2

LCMS m/z 530.9 (M+Na)⁺ (ES+).

### Step 3

¹H NMR (400 MHz, DMSO-d6) δ: 13.21 (br s, 1H), 7.63-7.59 (m, 2H), 7.37-7.35 (m, 2H), 6.73-6.63 (m, 2H), 2.62 (m, 4H), 1.99-1.90 (m, 1H), 1.76-1.65 (m, 1H).

### Intermediate 31: (E)-4-(1-(4-(difluoromethyl)phenyl)cyclobutoxy)-4-oxobut-2-enoic acid

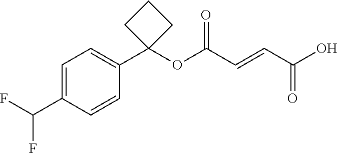 [SMILES: O=C(O)/C=C/C(=O)OC1(c2ccc(C(F)F)cc2)CCC1]

Prepared from 1-bromo-4-(difluoromethyl)benzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.56 (m, 4H), 6.65 (t, J=56.4 Hz, 1H), 2.60-2.53 (m, 2H), 2.43-2.35 (m, 2H), 2.09-2.02 (m, 1H), 1.76-1.71 (m, 1H).

### Step 2

LCMS m/z 497.0 (M+Na)⁺ (ES+).

### Step 3

LCMS m/z 319.3 (M+Na)⁺ (ES+). ¹H NMR (400 MHz, DMSO-d6) δ: 13.22 (br s, 1H), 7.63-7.56 (m, 4H), 7.03 (t, J=55.6 Hz, 1H), 6.73-6.63 (m, 2H), 2.63 (m, 4H), 2.00-1.94 (m, 1H), 1.78-1.68 (m, 1H).

### Intermediate 32: (E)-4-(3,3-difluoro-1-(4-(trifluoromethyl)phenyl)cyclobutoxy)-4-oxobut-2-enoic acid

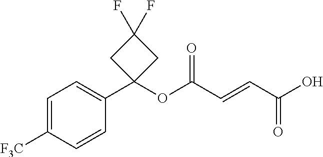 [SMILES: O=C(O)/C=C/C(=O)OC1(c2ccc(C(F)(F)F)cc2)CC(F)(F)C1]

Prepared from 1-iodo-4-(trifluoromethyl)benzene and 3,3-difluorocyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.67-7.63 (m, 2H), 7.62-7.25 (m, 2H), 3.20-3.01 (m, 4H).

### Step 2

LCMS m/z 550.8 (M+Na)⁺ (ES+).

### Step 3

¹H NMR (400 MHz, DMSO-d6) δ: 13.42 (br s, 1H), 7.78-7.71 (m, 4H), 6.77 (d, J=15.8 Hz, 1H), 6.66 (d, J=15.8 Hz, 1H), 3.50-3.33 (m, 4H).

### Intermediate 33: (E)-4-((cis)-3-methyl-1-(4-(trifluoromethyl)phenyl)cyclobutoxy)-4-oxobut-2-enoic acid

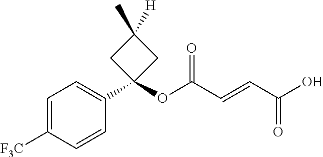 [SMILES: C[C@H]1C[C@](OC(=O)/C=C/C(=O)O)(c2ccc(C(F)(F)F)cc2)C1]

Prepared from 1-iodo-4-(trifluoromethyl)benzene and 3-methylcyclobutan-1-one using a similar procedure to (E)-4-(1-(5-bromopyridin-2-yl)cyclobutoxy)-4-oxobut-2-enoic acid.

### Step 1

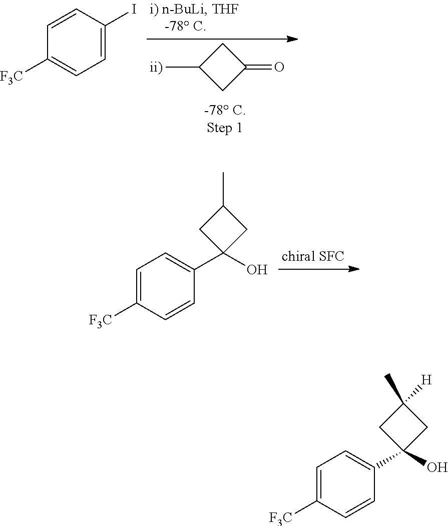

3-methyl-1-(4-(trifluoromethyl)phenyl)cyclobutan-1-ol (700 mg, 3.0 mmol) was separated by SFC (Column: CHIRALPAK AD-5 (30×250 mm 5 μm) (Daicel). Column temperature: 35° C. CO₂ flow rate: 36 mL/min; cosolvent flow rate: 9 mL/min; total flow rate: 45 mL/min. Cosolvent: methanol. Gradient: methanol 20%. Collection wavelength: 215 nm). Trans isomer (minor): Rt=0.898 min; cis isomer (major): Rt=1.039 min. The SFC fractions containing the cis isomer were concentrated under reduced pressure to to give (cis)-3-methyl-1-(4-(trifluoromethyl)phenyl)cyclobutan-1-ol (500 mg, 71% yield, stereochemistry arbitrarily assigned) which was used in Step 2. ¹H NMR (400 MHz, CDCl₃) δ: 7.65-7.60 (m, 4H), 2.80-2.69 (m, 2H), 2.14-1.97 (m, 3H), 1.20 (d, J=6.0 Hz, 3H).

### Step 2

LCMS m/z 528.9 (M+Na)⁺ (ES+).

### Step 3

¹H NMR (400 MHz, DMSO-d6) δ: 13.22 (br s, 1H), 7.75-7.69 (m, 4H), 6.73-6.63 (m, 2H), 2.85-2.77 (m, 2H), 2.27-2.06 (m, 3H), 1.15 (d, J=6.0 Hz, 3H).

### Intermediate 34: (E)-4-((cis)-3-cyano-1-(4-(trifluoromethyl)phenyl)cyclobutoxy)-4-oxobut-2-enoic acid

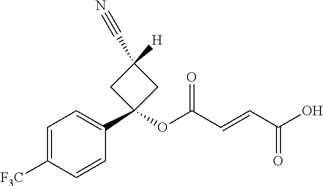 [SMILES: N#C[C@H]1C[C@](OC(=O)/C=C/C(=O)O)(c2ccc(C(F)(F)F)cc2)C1]

Prepared from 1-iodo-4-(trifluoromethyl)benzene and 3-oxocyclobutane-1-carbonitrile using a similar procedure to (E)-4-(1-(5-bromopyridin-2-yl)cyclobutoxy)-4-oxobut-2-enoic acid.

### Step 1

3-hydroxy-3-(4-(trifluoromethyl)phenyl)cyclobutane-1-carbonitrile was obtained as a single isomer (cis). ¹H NMR (400 MHz, CDCl₃) δ: 7.66 (d, J=8.4 Hz, 2H), 7.57 (d, J=8.0 Hz, 2H), 3.05-2.96 (m, 2H), 2.86-2.77 (m, 3H).

### Step 2

LCMS m/z 539.8 (M+Na)⁺ (ES+).

### Step 3

LCMS m/z 362.1 (M+Na)⁺ (ES+). ¹H NMR (400 MHz, DMSO-d6) δ: 13.31 (br s, 1H), 7.76 (d, J=8.4 Hz, 2H), 7.71 (d, J=8.3 Hz, 2H), 6.76 (d, J=15.8 Hz, 1H), 6.66 (d, J=15.8 Hz, 1H), 3.36-3.23 (m, 1H), 3.18-3.08 (m, 2H), 3.05-2.95 (m, 2H).

### Intermediate 35: (E)-4-oxo-4-((4-(4-(trifluoromethyl)phenyl)tetrahydro-2H-pyran-4-yl)oxy)but-2-enoic acid

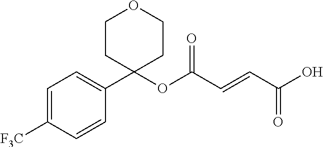 [SMILES: O=C(O)/C=C/C(=O)OC1(c2ccc(C(F)(F)F)cc2)CCOCC1]

Prepared from 1-iodo-4-(trifluoromethyl)benzene and dihydro-2H-pyran-4(3H)-one using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.66-7.60 (m, 4H), 3.97-3.89 (m, 4H), 2.19 (ddd, J=13.7, 12.0, 6.3 Hz, 2H), 1.70-1.63 (m, 2H).

### Step 2

LCMS m/z 523.0 (M+H)⁺ (ES+).

### Step 3

LCMS m/z 323.2 (M+Na)⁺ (ES+). ¹H NMR (400 MHz, DMSO-d6) δ: 13.28 (br s, 1H), 7.73 (d, J=8.3 Hz, 1H), 7.79-7.68 (m, 2H), 3.86-3.78 (m, 2H), 3.70 (td, J=11.7, 1.9 Hz, 2H), 2.38-2.30 (m, 2H), 2.19-2.07 (m, 2H).

### Intermediate 36: (E)-4-oxo-4-(1-(5-(trifluoromethyl)thiophen-2-yl)cyclobutoxy)but-2-enoic acid

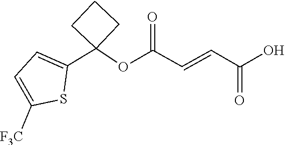 [SMILES: O=C(O)/C=C/C(=O)OC1(c2ccc(C(F)(F)F)s2)CCC1]

Prepared from 2-bromo-5-(trifluoromethyl)thiophene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.30-7.29 (m, 1H), 7.00-6.99 (m, 1H), 2.56-2.41 (m, 4H), 2.03-1.95 (m, 1H), 1.82-1.75 (m, 1H).

### Step 2

LCMS m/z 520.8 (M+Na)⁺ (ES+).

### Step 3

¹H NMR (400 MHz, DMSO-d6) δ: 13.10 (br s, 1H), 7.63-7.62 (m, 1H), 7.38-7.37 (m, 1H), 6.72 (d, J=15.8 Hz, 1H), 6.65 (d, J=15.8 Hz, 1H), 2.75-2.63 (m, 4H), 1.97-1.92 (m, 1H), 1.84-1.76 (m, 1H).

### Intermediate 37: 1-(4-(trifluoromethyl)phenyl)cyclopropan-1-ol

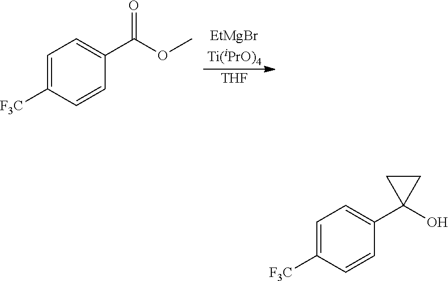

To the solution of methyl 4-(trifluoromethyl)benzoate (3 g, 14.7 mmol) and titanium tetraisopropoxide (5.8 g, 20.1 mmol) in THF (30 mL) was added ethyl magnesium bromide (15 mL, 45 mmol, 3M in ether) slowly at 0° C.; and the mixture was stirred at room temperature for 16 h. The reaction mixture was quenched with water (30 mL), and stirred for 1 h until a gray precipitate was formed. The solid was filtered off and filtrate extracted with tert-butyl methyl ether (3×40 mL). The combined organic layer was washed by brine, dried over Na₂SO₄, and concentrated under reduced pressure. The residue was purified column chromatography (40 g silica, 0-20% ethyl acetate/petroleum ether) to give a mixture (1:1) of 1-(4-(trifluoromethyl)phenyl)cyclopropan-1-ol and 1-(4-(trifluoromethyl)phenyl)propan-1-ol (1 g, 32% yield) as colorless oil. which was used in the next step directly. ¹H NMR (400 MHz, CDCl3) δ: 7.62-7.57 (m, 4H), 2.42 (s, 1H), 1.38-1.35 (m, 2H), 1.13-1.10 (m, 2H).

### Intermediate 38: (E)-4-oxo-4-(1-(4-(trifluoromethyl)phenyl)cyclopropoxy)but-2-enoic acid

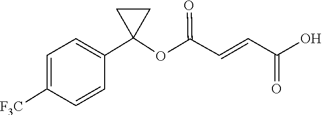 [SMILES: O=C(O)/C=C/C(=O)OC1(c2ccc(C(F)(F)F)cc2)CC1]

Prepared from 1-(4-(trifluoromethyl)phenyl)cyclopropan-1-ol using a similar procedure to (E)-4-(1-(5-bromopyridin-2-yl)cyclobutoxy)-4-oxobut-2-enoic acid (Step 2 and 3).

### Step 2

LCMS m/z 501.1 (M+Na)⁺ (ES+).

### Step 3

LCMS m/z 301.1 (M+H)⁺ (ES+). ¹H NMR (400 MHz, DMSO-d6) δ: 13.30 (br s, 1H), 7.67 (d, J=8.4 Hz, 2H), 7.41 (d, J=8.0 Hz, 2H), 6.77 (m, 2H), 1.49-1.39 (m, 4H).

### Intermediate 39: (E)-4-oxo-4-(1-(5-(trifluoromethyl)pyrimidin-2-yl)cyclobutoxy)but-2-enoic acid

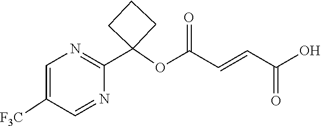 [SMILES: O=C(O)/C=C/C(=O)OC1(c2ncc(C(F)(F)F)cn2)CCC1]

Prepared from 2-iodo-5-(trifluoromethyl)pyrimidine and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 9.00 (s, 2H), 4.76 (s, 1H), 2.67-2.63 (m, 2H), 2.62-2.50 (m, 2H), 2.16-2.00 (m, 2H).

### Step 2

LCMS m/z 495.0 (M+H)⁺ (ES+).

### Step 3

LCMS m/z 317.2 (M+H)⁺ (ES+). ¹H NMR (400 MHz, DMSO-d6) δ: 9.02 (d, J=0.4 Hz, 2H), 7.05 (d, J=15.6 Hz, 1H), 6.87 (d, J=15.6 Hz, 1H), 2.94-2.87 (m, 2H), 2.72-2.64 (m, 2H), 2.20-2.12 (m, 2H).

### Intermediate 40: (E)-4-(1-(3,5-dimethoxyphenyl)cyclobutoxy)-4-oxobut-2-enoic acid

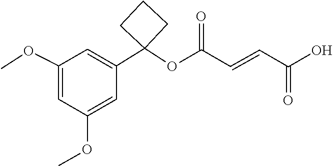 [SMILES: COc1cc(OC)cc(C2(OC(=O)/C=C/C(=O)O)CCC2)c1]

Prepared from 1-bromo-3,5-dimethoxybenzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 6.65 (d, J=2.4 Hz, 2H), 6.38 (t, J=2.0 Hz, 1H), 3.81 (s, 6H), 2.50-2.57 (m, 2H), 2.38-2.31 (m, 2H), 2.03-2.00 (m, 2H), 1.73-1.68 (m, 1H).

### Step 2

LCMS m/z 506.9 (M+Na)⁺ (ES+).

### Step 3

LCMS m/z 329.2 (M+Na)⁺ (ES+). ¹H NMR (400 MHz, DMSO-d6) δ: 13.20 (br s, 1H), 6.67 (s, 2H), 6.55 (d, J=2.0 Hz, 2H), 6.43 (d, J=2.0 Hz, 1H), 3.74 (s, 6H), 2.59-2.55 (m, 4H), 1.95-1.92 (m, 1H), 1.76-1.71 (m, 1H).

### Intermediate 41: (E)-4-(1-(3-chloro-5-(trifluoromethoxy)phenyl)cyclobutoxy)-4-oxobut-2-enoic acid

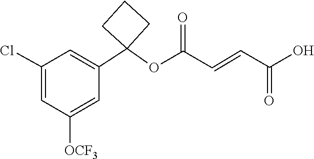 [SMILES: O=C(O)/C=C/C(=O)OC1(c2cc(Cl)cc(OC(F)(F)F)c2)CCC1]

Prepared from 1-bromo-3-chloro-5-(trifluoromethyl)benzene and cyclobutanone using a similar procedure to (E)-4-(1-(5-bromopyridin-2-yl)cyclobutoxy)-4-oxobut-2-enoic acid.

### Step 1

¹H NMR (400 MHz, CDCl₃) δ: 7.67 (s, 1H), 7.57 (s, 1H), 7.50 (s, 1H), 2.62-2.56 (m, 2H), 2.50-2.41 (m, 2H), 2.21-2.02 (m, 1H), 1.82-1.68 (m, 1H).

### Step 2

LCMS m/z 548.8 (M+Na)⁺ (ES+).

### Step 3

¹H NMR (400 MHz, DMSO-d6) δ: 13.26 (br s, 1H), 7.86 (s, 1H), 7.82 (s, 1H), 7.72 (s, 1H), 6.77-6.62 (m, 2H), 2.73-2.58 (m, 4H), 2.02-1.95 (m, 1H), 1.78-1.71 (m, 1H).

### Intermediate 42 and Intermediate 43: (S)-2,2,2-trifluoro-1-(4-(trifluoromethyl)phenyl)ethan-1-ol and (R)-2,2,2-trifluoro-1-(4-(trifluoromethyl)phenyl)ethan-1-ol

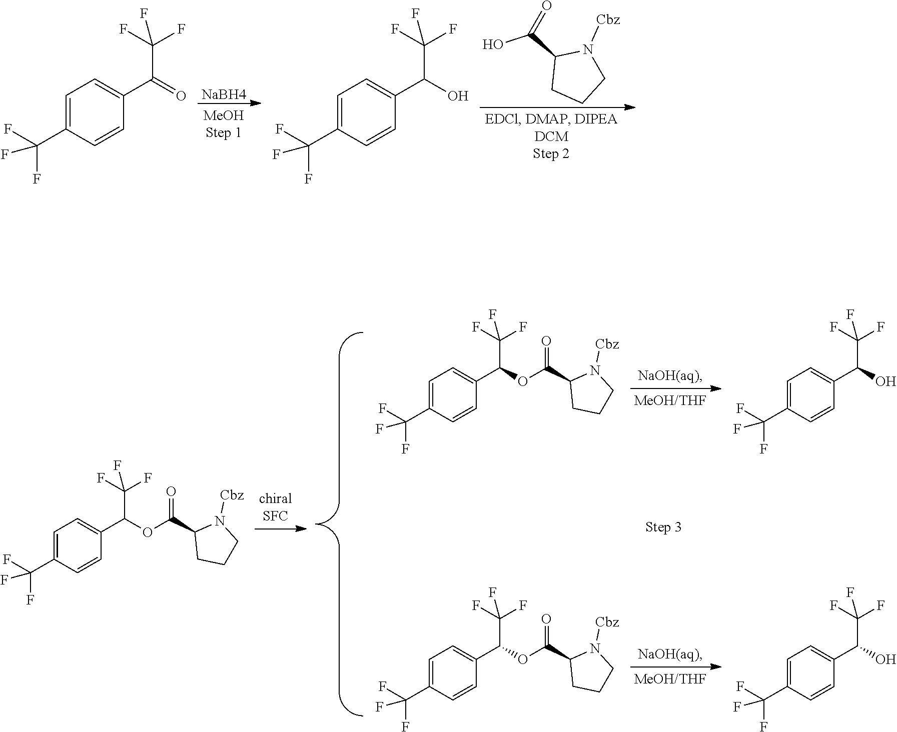

### Step 1

To a solution of 2,2-dimethylcyclohexanone (2.0 g) in MeOH (40 mL) was added NaBH₄ (628 mg, 16.52 mmol) at 0° C., and the resulting mixture was stirred at 0° C. for 2 h. The reaction mixture was quenched with 2M HCl and concentrated under reduced pressure. The residue was extracted with MTBE (3×40 mL). The combined organic layers were washed with brine and dried over Na₂SO₄. The filtrate was concentrated under reduced pressure and the residue was purified by flash column chromatography (0-20% tert-Butyl methyl ether/petroleum ether) to give 2,2,2-trifluoro-1-(4-(trifluoromethyl)phenyl) ethanol (1.20 g, 60% yield) as a colorless oil. ¹H NMR (400 MHz, CDCl₃) δ: 7.70-7.62 (m, 4H), 5.11 (q, J=6.8 Hz, 1H).

### Step 2

To a solution of 2,2,2-trifluoro-1-(4-(trifluoromethyl)phenyl)ethanol (1.20 g, 4.92 mmol), (S)-1-(benzyloxycarbonyl)pyrrolidine-2-carboxylic acid (1.84 g, 7.38 mmol), DMAP (720 mg, 5.9 mmol) and DIPEA (1.90 g, 14.76 mmol) in DCM (18 mL) was added EDCI (1.41 g, 7.38 mmol) at 0° C., and the resulting light yellow mixture was stirred at room temperature for 2 h. The mixture was quenched with 0.5 N HCl (10 mL), the organic phase separated and the aquoes phase extracted with DCM (2×20 mL). The combined organic layers were washed with brine and dried over Na₂SO₄. The filtrate was concentrated under reduced pressure and the residue was purified by flash column chromatography (0-20% tert-Butyl methyl ether/petroleum ether) to give (2S)-1-benzyl 2-(2,2,2-trifluoro-1-(4-(trifluoromethyl)phenyl)ethyl) pyrrolidine-1,2-dicarboxylate (1.60 g, 68% yield) as colorless oil. LCMS m/z 476.2 (M+H)⁺ (ES+). (2S)-1-benzyl 2-(2,2,2-trifluoro-1-(4-(trifluoromethyl)phenyl)ethyl) pyrrolidine-1,2-dicarboxylate (1.60 g, 3.36 mmol) was separated by SFC (Column: CHIRALPAK AD-5(30×250 mm 5 μm) (Daicel). Column temperature: 35° C. Flow Rate: CO₂ flow Rate: 36 mL/min; co solvent flow rate: 9 mL/min; total flow rate: 45 mL/min. Co solvent: iso-propanol. Gradient: iso-propanol 20%. Collection wavelength: 215 nm). The SFC fractions were concentrated under reduced pressure to remove isopropanol to give (S)-1-benzyl 2-((2,2,2-trifluoro-1-(4-(trifluoromethyl)phenyl)ethyl) pyrrolidine-1,2-dicarboxylate ISOMER 1 (750 mg, 100% ee, 47% yield) and (S)-1-benzyl 2-((2,2,2-trifluoro-1-(4-(trifluoromethyl)phenyl)ethyl) pyrrolidine-1,2-dicarboxylate ISOMER 2 (720 mg, 97.8% ee, 45% yiled). (R) or (S) configuration were assigned arbitrarily. Chiral HPLC: (Column: CHIRALPAKAD-3 (4.6×100 mm); Flow Rate: 2 mL/min; Cosolvent: 15% isopropanol; collection wavelength: 200-400 nm) (S) isomer Rt=0.904 min; (R) isomer Rt=1.108 min.

### Step 3

A solution of (S)-1-benzyl 2-(2,2,2-trifluoro-1-(4-(trifluoromethyl)phenyl)ethyl) pyrrolidine-1,2-dicarboxylate ISOMER 1 (750 mg, 1.58 mmol) and NaOH (126 mg, 3.16 mmol) in MeOH/THF (5 mL/2.5 mL) was stirred at room temperature for 12 h. The mixture was concentrated under reduced pressure, quenched with 0.5 N HCl and extracted with EtOAc (2×5 mL). The combined organic layers were washed with brine and dried over Na₂SO₄ and concentrated under reduced pressure to give 2,2,2-trifluoro-1-(4-(trifluoromethyl)phenyl)ethanol ISOMER 1 (350 mg, 90% yield) as yellow solid. 2,2,2-trifluoro-1-(4-(trifluoromethyl)phenyl)ethanol ISOMER 2 (330 mg, 89% yield) was obtained as yellow solid using a similar procedure.

### Intermediate 44: (E)-4-oxo-4-(2,2,2-trifluoro-1-(4-(trifluoromethyl)phenyl)ethoxy)but-2-enoic acid

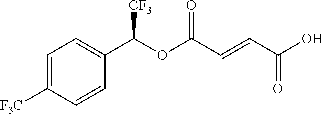 [SMILES: O=C(O)/C=C/C(=O)O[C@@H](c1ccc(C(F)(F)F)cc1)C(F)(F)F]

Prepared from 2,2,2-trifluoro-1-(4-(trifluoromethyl)phenyl)ethanol ISOMER 1 using a similar procedure to (E)-4-(1-(5-bromopyridin-2-yl)cyclobutoxy)-4-oxobut-2-enoic acid (Step 2 and 3 only).

### Step 2

LCMS m/z 542.8 (M+Na)⁺ (ES+).

### Step 3

¹H NMR (400 MHz, DMSO-d6) δ: 13.41 (s, 1H), 7.86 (s, 4H), 6.95-6.77 (m, 3H).

### Intermediate 45: (E)-4-oxo-4-(2,2,2-trifluoro-1-(4-(trifluoromethyl)phenyl)ethoxy)but-2-enoic acid

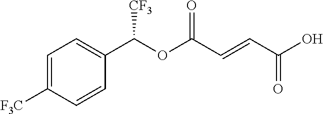 [SMILES: O=C(O)/C=C/C(=O)O[C@H](c1ccc(C(F)(F)F)cc1)C(F)(F)F]

Prepared from 2,2,2-trifluoro-1-(4-(trifluoromethyl)phenyl)ethanol ISOMER 2 using a similar procedure to (E)-4-(1-(5-bromopyridin-2-yl)cyclobutoxy)-4-oxobut-2-enoic acid (Step 2 and 3 only).

### Step 2

LCMS m/z 542.7 (M+Na)⁺ (ES+).

### Step 3

¹H NMR (400 MHz, DMSO-d6) δ: 13.41 (s, 1H), 7.86 (s, 4H), 6.92-6.77 (m, 3H).

### Example 1: (E)-2-((4-(cyclooctyloxy)-4-oxobut-2-enoyl)oxy)acetic acid

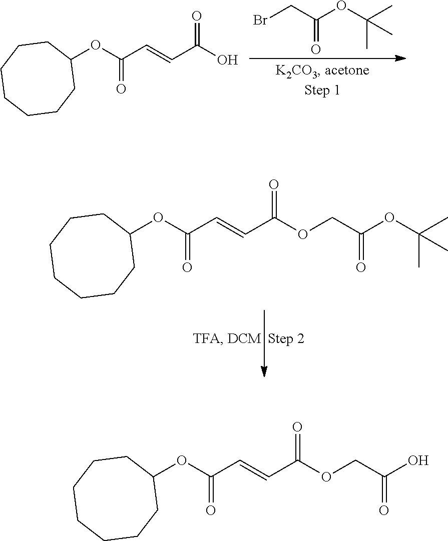

### Step 1

To a solution of crude (E)-4-(cyclooctyloxy)-4-oxobut-2-enoic acid (Intermediate 6, the synthesis of which is described in Example 3, 115 g, 508 mmol) in acetone (690 mL) was added tert-butyl 2-bromoacetate (99.1 g, 508 mmol) and K₂CO₃ (140 g, 1.02 mol). The mixture was stirred at 15° C. for 12 h, then the mixture was added into water (800 mL) and extracted with EtOAc (3×600 mL). The combined organic layers were washed with brine (500 mL), dried over Na₂SO₄, filtered and concentrated under reduced pressure. The residue was purified by column chromatography (SiO₂, 2-20% EtOAc/petroleum ether) to give 2-(tert-butoxy)-2-oxoethyl cyclooctyl fumarate (80.0 g, 235 mmol, 46%) as a yellow oil. ¹H NMR (400 MHz, DMSO) δ: 6.83-6.72 (m, 2H), 5.00-4.94 (m, 1H), 4.69 (s, 2H), 1.81-1.42 (m, 23H).

### Step 2

To a mixture of 2-(tert-butoxy)-2-oxoethyl cyclooctyl fumarate (80.0 g, 235 mmol) in DCM (240 mL) was added TFA (240 mL). The mixture was degassed and purged with N₂ three times, and then stirred at 20° C. for 2 h under a N₂ atmosphere. The reaction mixture was added into ice water (50 mL), and the organic phase was extracted with dichloromethane (2×20 mL). The combined organic phases were washed with brine (30 mL), dried with anhydrous Na₂SO₄, filtered and concentrated under reduced pressure. The residue was purified by column chromatography (SiO₂, 1-100% MeOH/dichloromethane) to give the crude product which still contained TFA and so was lyophilised to give (E)-2-((4-(cyclooctyloxy)-4-oxobut-2-enoyl)oxy)acetic acid (50.5 g, 178 mmol, 76%) as an off-white solid. LCMS m/z 283 (M−H)⁻ (API⁻). ¹H NMR (400 MHz, CDCl₃) δ: 8.77 (br. s, 1H), 6.99-6.87 (m, 2H), 5.10-5.04 (m, 1H), 4.79 (s, 2H), 1.84-1.79 (m, 6H), 1.59-1.53 (m, 8H).

### Example 2: (E)-2-((4-(cyclohexyloxy)-4-oxobut-2-enoyl)oxy)acetic acid

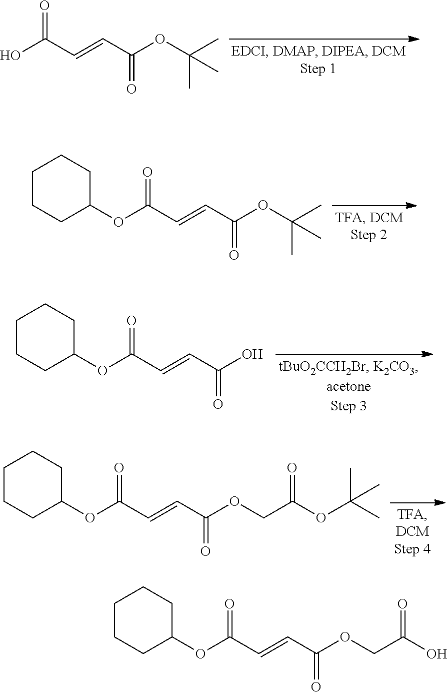 [SMILES: CC(C)(C)OC(=O)/C=C/C(=O)O ; CC(C)(C)OC(=O)/C=C/C(=O)OC1CCCCC1 ; O=C(O)/C=C/C(=O)OC1CCCCC1 ; CC(C)(C)OC(=O)COC(=O)/C=C/C(=O)OC1CCCCC1 ; O=C(O)COC(=O)/C=C/C(=O)OC1CCCCC1]

### Step 1

EDCI (2.12 g, 11 mmol) was added to a mixture of (E)-4-(tert-butoxy)-4-oxobut-2-enoic acid (1.00 g, 5.52 mmol), DIPEA (1.9 mL, 11 mmol), DMAP (0.067 g, 0.55 mmol) and cyclohexanol (0.58 mL, 5.52 mmol) in DCM (30 mL). The mixture was stirred at RT for 16 h, then concentrated onto silica and purified by chromatography on silica gel (0-20% EtOAc/DCM) to afford tert-butyl cyclohexyl fumarate (920 mg, 3.55 mmol) as a clear colourless oil. LCMS m/z 254.7 (M+H)⁺ (ES⁺). ¹H NMR (400 MHz, DMSO) δ 6.64 (d, J=1.3 Hz, 2H), 4.89-4.66 (m, 1H), 1.84-1.77 (m, 2H), 1.71-1.60 (m, 2H), 1.54-1.19 (m, 15H).

### Step 2

TFA (8 mL, 104 mmol) was added to a solution of tert-butyl cyclohexyl fumarate (920 mg, 3.55 mmol) in DCM (8 mL) at RT. The reaction mixture was stirred for 1 h, diluted with toluene (50 mL) and concentrated. The residue was co-evaporated with toluene (2×20 mL), then taken up in EtOAc (100 mL), washed with brine (50 mL), dried (MgSO₄) and concentrated to afford (E)-4-(cyclohexyloxy)-4-oxobut-2-enoic acid (680 mg, 3.40 mmol) as a colourless solid. LCMS m/z 196.7 (M−H)⁻ (ES⁻). ¹H NMR (400 MHz, DMSO) δ 13.20 (s, 1H), 6.92-5.83 (m, 2H), 5.02-4.46 (m, 1H), 1.96-1.04 (m, 10H).

### Step 3

Tert-butyl bromoacetate (0.22 mL, 1.46 mmol) was added to a mixture of (E)-4-(cyclohexyloxy)-4-oxobut-2-enoic acid (340 mg, 1.72 mmol) and potassium carbonate (308 mg, 2.23 mmol) in acetone (10 mL). The reaction mixture was stirred for 16 h at RT. The mixture was diluted with EtOAc (20 mL), filtered and concentrated. The residue was taken up in EtOAc (100 mL) and washed with sat. aq. NaHCO₃ (3×50 mL). The organic layer was dried (MgSO₄) and concentrated. The crude product was purified by chromatography on silica gel (0-20% EtOAc/DCM) to afford 2-(tert-butoxy)-2-oxoethyl cyclohexyl fumarate (486 mg, 1.54 mmol) as a colourless oil. LCMS m/z 335.2 (M+Na)⁺ (ES⁺). ¹H NMR (400 MHz, DMSO) δ 6.88-6.54 (m, 2H), 4.88-4.77 (m, 1H), 4.69 (s, 2H), 1.88-1.78 (m, 2H), 1.74-1.51 (m, 2H), 1.56-1.11 (m, 15H).

### Step 4

TFA (5 mL, 65 mmol) was added to a solution of 2-(tert-butoxy)-2-oxoethyl cyclohexyl fumarate (486 mg, 1.54 mmol) in DCM (5 mL) at RT. The reaction mixture was stirred for 1 h, then diluted with toluene (50 mL) and concentrated. The residue was taken up in EtOAc (100 mL), washed with brine (50 mL), dried (MgSO₄) and concentrated to afford (E)-2-((4-(cyclohexyloxy)-4-oxobut-2-enoyl)oxy)acetic acid (312 mg, 1.21 mmol) as a colourless solid. LCMS m/z 254.8 (M−H)⁻ (ES⁻). ¹H NMR (400 MHz, DMSO) δ 6.81 (s, 2H), 4.86-4.74 (m, 1H), 4.71 (s, 2H), 1.85-1.76 (m, 2H), 1.73-1.60 (m, 2H), 1.52-1.16 (m, 6H) (1 exchangeable proton not observed).

### Example 3: (E)-3-((4-(Cyclooctyloxy)-4-oxobut-2-enoyl)oxy)propanoic acid

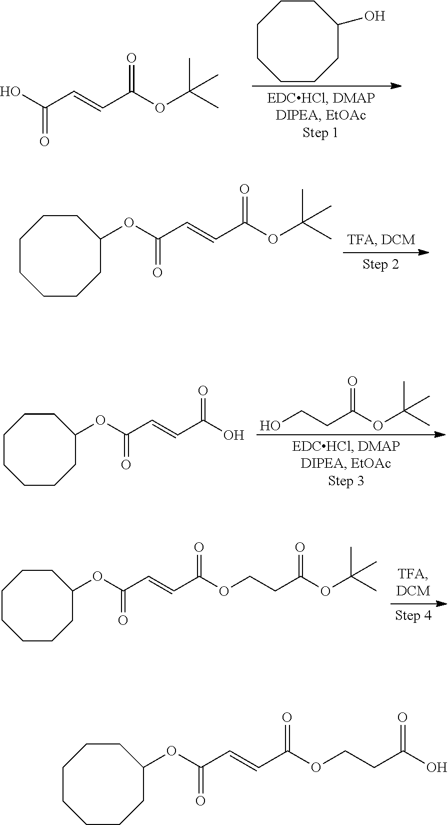

### Step 1

A mixture of cyclooctanol (177 g, 1.38 mol), (E)-4-(tert-butoxy)-4-oxobut-2-enoic acid (238 g, 1.38 mol), DMAP (16.9 g, 138 mmol) and DIPEA (357 g, 2.76 mol) in EtOAc (1.43 L) was degassed and purged with N₂ three times. EDC·HCl (530 g, 2.76 mol) was added into the mixture, and the mixture was stirred at 20° C. for 12 h under a N₂ atmosphere. The mixture was then added into water (1.50 L), and the mixture was extracted with EtOAc (2×1.00 L). The combined organic layers were washed with brine (500 mL), dried over Na₂SO₄, filtered and concentrated under reduced pressure. The residue was purified by column chromatography (SiO₂, 2-20% EtOAc/petroleum ether) to give tert-butyl cyclooctyl fumarate (245 g, 868 mmol, 63%) as a yellow oil. ¹H NMR (400 MHz, DMSO) δ: 6.72-6.58 (m, 2H), 4.97-4.91 (m, 1H), 1.79-1.38 (m, 23H).

### Step 2

To a solution of tert-butyl cyclooctyl fumarate (142 g, 503 mmol) in DCM (426 mL) was added TFA (656 g, 5.75 mol), and the mixture was stirred at 20° C. for 2 h. The mixture was concentrated under reduced pressure and the residue was diluted with water (600 mL) and extracted with EtOAc (3×600 mL). The combined organic layers were washed with brine (500 mL), dried over Na₂SO₄, filtered and concentrated under reduced pressure to give crude (E)-4-(cyclooctyloxy)-4-oxobut-2-enoic acid (Intermediate 6, 135 g, >100%, crude) as a grey oil. ¹H NMR (400 MHz, DMSO) δ: 6.72-6.62 (m, 2H), 4.97-4.91 (m, 1H), 1.79-1.50 (m, 14H). One exchangeable proton not observed.

### Step 3

To a solution of crude (E)-4-(cyclooctyloxy)-4-oxobut-2-enoic acid (Intermediate 6, 40.0 g, 177 mmol) in EtOAc (240 mL) was added DMAP (2.16 g, 17.7 mmol), DIPEA (45.7 g, 354 mmol) and tert-butyl 3-hydroxypropanoate (25.8 g, 177 mmol), followed by EDC·HCl (67.8 g, 354 mmol). The mixture was stirred at 15° C. for 12 h, then the mixture was added into water (300 mL) and extracted with EtOAc (3×300 mL). The combined organic layers were washed with brine (200 mL), dried over Na₂SO₄, filtered and concentrated under reduced pressure. The residue was purified by column chromatography (SiO₂, 2-20% EtOAc/petroleum ether) to give 3-(tert-butoxy)-3-oxopropyl cyclooctyl fumarate (36.0 g, 102 mmol, 57%) as a white oil. ¹H NMR (400 MHz, DMSO) δ: 6.81-6.67 (m, 2H), 4.99-4.93 (m, 1H), 4.33 (t, J=6.0 Hz, 2H), 2.63 (t, J=6.0 Hz, 2H), 1.80-1.40 (m, 23H).

### Step 4

To a solution of 3-(tert-butoxy)-3-oxopropyl cyclooctyl fumarate (31.0 g, 87.5 mmol) in DCM (93.0 mL) was added TFA (93.0 mL) and the mixture was degassed and purged with N₂ three times, and then stirred at 20° C. for 2 h under a N₂ atmosphere. The reaction mixture was poured to ice water (50 mL), and the organic phase was extracted with dichloromethane (2×20 mL). The combined organic phases were washed with brine (30 mL), dried with anhydrous Na₂SO₄, filtered and concentrated under reduced pressure. The residue was purified by column chromatography (SiO₂, 1-100% MeOH/dichloromethane) to give the crude product which still contained TFA and so was lyophilised to give (E)-3-((4-(cyclooctyloxy)-4-oxobut-2-enoyl)oxy)propanoic acid (10.1 g, 33.9 mmol, 39%) as an off-white solid. LCMS m/z 619 (2M+Na)⁺ (API⁺). ¹H NMR (400 MHz, CDCl₃) δ: 6.88-6.79 (m, 2H), 5.09-5.02 (m, 1H), 4.48 (t, J=6.0 Hz, 2H), 2.78 (t, J=6.0 Hz, 2H), 1.85-1.71 (m, 6H), 1.59-1.55 (m, 8H). One exchangeable proton not observed.

### Example 4: (E)-3-((4-(cyclohexyloxy)-4-oxobut-2-enoyl)oxy)propanoic acid

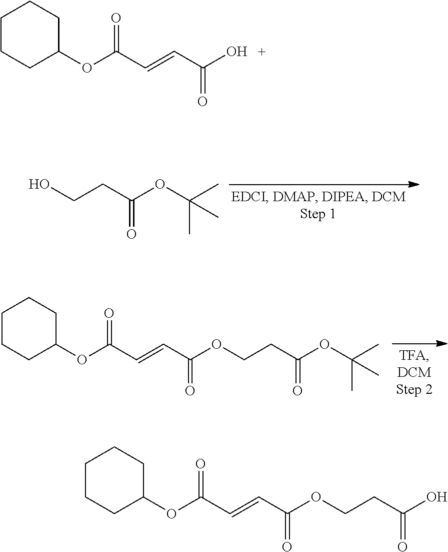

### Step 1

EDCI (658 mg, 3.43 mmol) was added to a solution of (E)-4-(cyclohexyloxy)-4-oxobut-2-enoic acid (340 mg, 1.72 mmol), tert-butyl 3-hydroxypropanoate (0.25 mL, 1.72 mmol), DIPEA (0.6 mL, 3.4 mmol), and DMAP (21 mg, 0.17 mmol) in DCM (16 mL) at RT. The mixture was stirred at RT for 16 h. The reaction mixture was concentrated and purified by chromatography on silica gel (0-20% EtOAc/DCM) to afford 3-(tert-butoxy)-3-oxopropyl cyclohexyl fumarate (357 mg, 1.08 mmol) as a clear colourless oil. LCMS m/z 348.8 (M+Na)⁺ (ES⁺). ¹H NMR (400 MHz, DMSO) δ 6.72 (d, J=1.1 Hz, 2H), 4.85-4.74 (m, 1H), 4.32 (t, J=6.5 Hz, 2H), 2.62 (t, J=5.5 Hz, 2H), 1.93-1.15 (m, 19H).

### Step 2

TFA (3.4 mL, 44 mmol) was added to a solution of 3-(tert-butoxy)-3-oxopropyl cyclohexyl fumarate (357 mg, 1.08 mmol) in DCM (3 mL) at RT. The mixture was stirred for 1 h at RT, diluted with toluene (20 mL) and concentrated. The residue was taken up in EtOAc (50 mL) and washed with brine (20 mL), dried (MgSO₄) and concentrated to afford (E)-3-((4-(cyclohexyloxy)-4-oxobut-2-enoyl)oxy)propanoic acid (132 mg, 0.48 mmol) as a colourless solid. LCMS m/z 293.2 (M+Na)⁺ (ES⁺). ¹H NMR (400 MHz, DMSO) δ 12.44 (s, 1H), 6.72 (s, 2H), 4.85-4.70 (m, 1H), 4.33 (t, J=6.1 Hz, 2H), 2.64 (t, J=6.1 Hz, 2H), 1.87-1.76 (m, 2H), 1.74-1.57 (m, 2H), 1.54-1.10 (m, 6H).

### Example 5: 2-(1H-tetrazol-5-yl)ethyl cyclooctyl fumarate

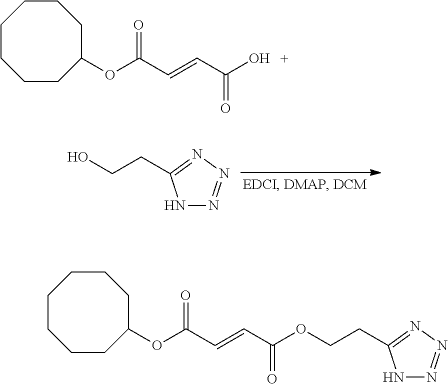 [SMILES: O=C(O)/C=C/C(=O)OC1CCCCCCC1 ; OCCc1nnn[nH]1 ; O=C(/C=C/C(=O)OC1CCCCCCC1)OCCc1nnn[nH]1]

A slurry of EDCI (345 mg, 1.8 mmol) in DCM (3 mL) was added slowly to a solution of (E)-4-(cyclooctyloxy)-4-oxobut-2-enoic acid (Intermediate 6, 272 mg, 1.2 mmol), 2-(1H-tetrazol-5-yl)ethanol (164 mg, 1.44 mmol) and DMAP (220 mg, 1.8 mmol) in DCM (3 mL) at 0° C. The mixture was allowed to warm slowly to RT and stirred for 18 h. The reaction mixture was diluted with 1 M HCl (5 mL) and the phases were separated. The aqueous phase was extracted with DCM (2×5 mL). The combined organic phases were dried (MgSO₄) and concentrated. The crude product was purified by chromatography on silica gel (0-100% EtOAc/isohexane) to afford 2-(1H-tetrazol-5-yl)ethyl cyclooctyl fumarate (183 mg, 0.56 mmol) as a white solid. LCMS m/z 323.2 (M+H)⁺ (ES⁺). ¹H NMR (400 MHz, DMSO) δ 6.72 (s, 2H), 4.99-4.92 (m, 1H), 4.50 (t, J=6.2 Hz, 2H), 3.32 (t, J=6.2 Hz, 2H), 1.85-1.43 (m, 14H) (1 exchangeable proton not observed).

### Example 6: (S,E)-2-((4-(cyclooctyloxy)-4-oxobut-2-enoyl)oxy)propanoic acid

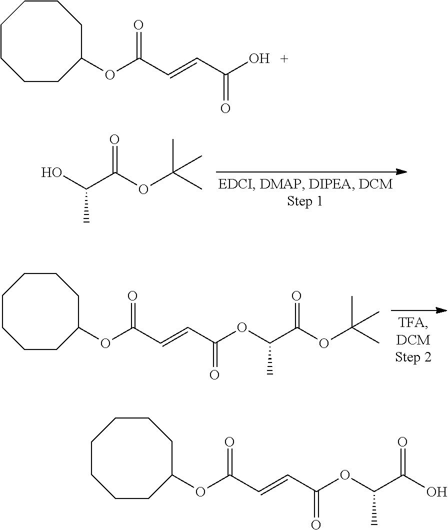

Prepared using a similar procedure to (E)-3-((4-(cyclohexyloxy)-4-oxobut-2-enoyl)oxy)propanoic acid using (S)-tert-butyl 2-hydroxypropanoate.

### Step 1

LCMS m/z 377.4 (M+Na)⁺ (ES⁺). ¹H NMR (400 MHz, DMSO) δ 6.80 (d, J=17.3 Hz, 1H), 6.76 (d, J=17.1 Hz, 1H), 5.02-4.92 (m, 2H), 1.86-1.45 (m, 14H), 1.43 (d, J=7.0 Hz, 3H), 1.40 (s, 9H).

### Step 2

LCMS m/z 321.2 (M+Na)⁺ (ES⁺). ¹H NMR (400 MHz, DMSO) δ 13.19 (s, 1H), 6.80 (d, J=16.6 Hz, 1H), 6.75 (d, J=16.6 Hz, 1H), 5.04 (q, J=7.1 Hz, 1H), 4.96 (tt, J=8.2, 4.3 Hz, 1H), 1.87-1.38 (m, 17H).

### Example 7: (E)-3-((4-(cyclooctyloxy)-4-oxobut-2-enoyl)oxy)-2,2-dimethylpropanoic acid

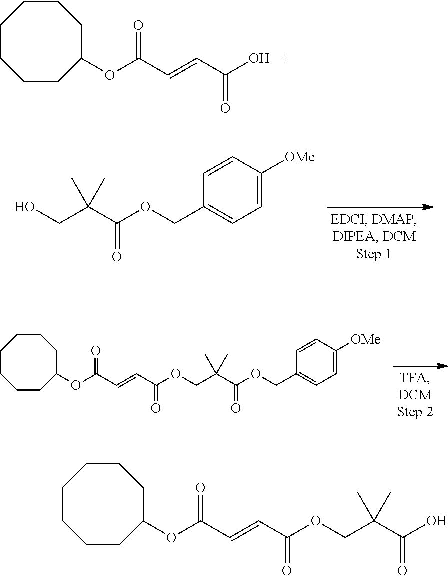 [SMILES: O=C(O)/C=C/C(=O)OC1CCCCCCC1 ; COc1ccc(COC(=O)C(C)(C)CO)cc1 ; COc1ccc(COC(=O)C(C)(C)COC(=O)/C=C/C(=O)OC2CCCCCCC2)cc1 ; CC(C)(COC(=O)/C=C/C(=O)OC1CCCCCCC1)C(=O)O]

### Step 1

EDCI (0.424 g, 2.21 mmol) was added to a solution of (E)-4-(cyclooctyloxy)-4-oxobut-2-enoic acid (Intermediate 6, 0.25 g, 1.10 mmol), 4-methoxybenzyl 3-hydroxy-2,2-dimethylpropanoate (0.263 g, 1.11 mmol), DIPEA (0.39 mL, 2.2 mmol) and DMAP (0.013 g, 0.11 mmol) in DCM (5 mL) at RT. The mixture was stirred at RT for 18 h, then concentrated. The crude product was purified by chromatography on silica gel (0-20% EtOAc/isohexane) to afford cyclooctyl (3-((4-methoxybenzyl)oxy)-2,2-dimethyl-3-oxopropyl) fumarate (0.265 g, 0.58 mmol) as a clear colourless oil. LCMS m/z 469.2 (M+Na)⁺ (ES⁺). ¹H NMR (400 MHz, DMSO) δ 7.30-7.21 (m, 2H), 6.91-6.84 (m, 2H), 6.62 (d, J=17.6 Hz, 1H), 6.57 (d, J=17.5 Hz, 1H), 5.04 (s, 2H), 4.95 (tt, J=8.2, 4.2 Hz, 1H), 4.18 (s, 2H), 3.72 (s, 3H), 1.88-1.38 (m, 14H), 1.19 (s, 6H).

### Step 2

TFA (0.14 mL, 1.78 mmol) was added dropwise to a solution of cyclooctyl (3-((4-methoxybenzyl)oxy)-2,2-dimethyl-3-oxopropyl) fumarate (0.265 g, 0.59 mmol) in DCM (6 mL). The reaction was slowly allowed to warm to RT and the mixture was stirred at RT for 18 h. The mixture was concentrated and the residue co-evaporated with toluene (2×10 mL). The crude product was purified by chromatography on silica gel (0-10% MeOH/DCM) 1 to afford (E)-3-((4-(cyclooctyloxy)-4-oxobut-2-enoyl)oxy)-2,2-dimethylpropanoic acid (0.165 g, 0.495 mmol) as a slightly yellow oil. LCMS m/z 349.1 (M+Na)⁺ (ES⁺). ¹H NMR (400 MHz, DMSO) δ 12.50 (s, 1H), 6.74 (d, J=18.5 Hz, 1H), 6.69 (d, J=18.5 Hz, 1H), 4.96 (tt, J=8.2, 4.3 Hz, 1H), 4.17 (s, 2H), 1.92-1.35 (m, 14H), 1.16 (s, 6H).

### Example 8: (E)-1-((4-(cyclooctyloxy)-4-oxobut-2-enoyl)oxy)cyclopropane-1-carboxylic acid

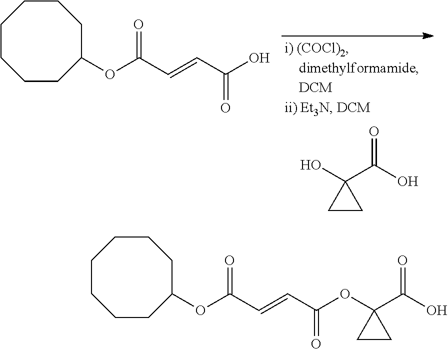 [SMILES: O=C(O)/C=C/C(=O)OC1CCCCCCC1 ; O=C(O)C1(O)CC1 ; O=C(/C=C/C(=O)OC1(C(=O)O)CC1)OC1CCCCCCC1]

Oxalyl chloride (0.23 mL, 2.6 mmol) was added to a solution of (E)-4-(cyclooctyloxy)-4-oxobut-2-enoic acid (Intermediate 6, 0.20 g, 0.85 mmol) and dimethylformamide (1 drop) in DCM (5 mL) at 0° C. The mixture was warmed to RT, stirred for 2.5 h and concentrated. The residue was taken up in DCM (5 mL) and cooled to 0° C. 1-Hydroxycyclopropanecarboxylic acid (0.102 g, 1.0 mmol) and triethylamine (0.54 mL, 3.87 mmol were added and the mixture was warmed to RT and stirred for 18 h. 1M HCl (30 mL) was added and the mixture extracted with DCM (3×30 mL). The combined organic extracts were dried (phase separator) and concentrated. The crude product was purified by chromatography on RP Flash C18 (5-100% MeCN/Water, 0.1% Formic Acid) to afford (E)-1-((4-(cyclooctyloxy)-4-oxobut-2-enoyl)oxy)cyclopropanecarboxylic acid (0.117 g, 0.36 mmol) as a white solid. LCMS m/z 333.2 (M+Na)⁺ (ES⁺). ¹H NMR (400 MHz, DMSO) δ 13.09 (s, 1H), 6.84-6.68 (m, 2H), 5.03-4.91 (m, 1H), 1.88-1.38 (m, 16H), 1.32-1.24 (m, 2H).

### Example 9: (E)-2-((4-oxo-4-(spiro[3.3]heptan-2-yloxy)but-2-enoyl)oxy)acetic acid

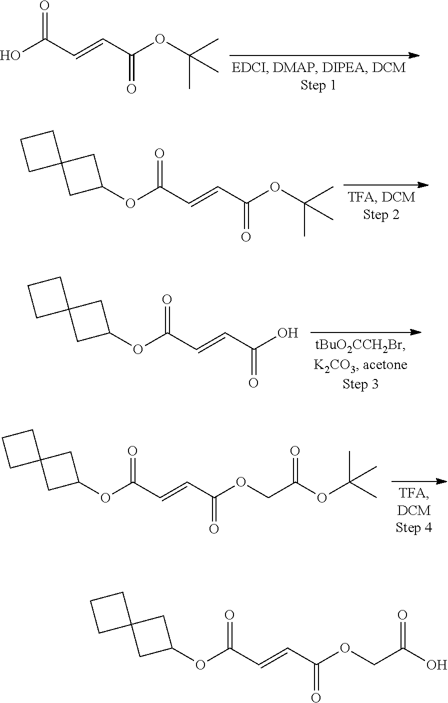 [SMILES: CC(C)(C)OC(=O)/C=C/C(=O)O ; CC(C)(C)OC(=O)/C=C/C(=O)OC1CC2(CCC2)C1 ; O=C(O)/C=C/C(=O)OC1CC2(CCC2)C1 ; CC(C)(C)OC(=O)COC(=O)/C=C/C(=O)OC1CC2(CCC2)C1 ; O=C(O)COC(=O)/C=C/C(=O)OC1CC2(CCC2)C1]

Prepared using a similar procedure to (E)-2-((4-(cyclohexyloxy)-4-oxobut-2-enoyl)oxy)acetic acid.

### Step 1

¹H NMR (400 MHz, DMSO) δ 6.64 (s, 2H), 4.96-4.84 (m, 1H), 2.48-2.41 (m, 2H), 2.07-1.92 (m, 6H), 1.84-1.75 (m, 2H), 1.46 (s, 9H).

### Step 2, Intermediate 7: (E)-4-oxo-4-(spiro[3.3]heptan-2-yloxy)but-2-enoic acid

LCMS m/z 209.1 (M−H)⁻ (ES⁻). ¹H NMR (400 MHz, DMSO) δ 13.21 (s, 1H), 6.75-6.58 (m, 2H), 4.97-4.83 (m, 1H), 2.49-2.41 (m, 2H), 2.08-1.93 (m, 6H), 1.85-1.75 (m, 2H).

### Step 3

¹H NMR (400 MHz, DMSO) δ 6.81 (d, J=1.9 Hz, 2H), 4.97-4.86 (m, 1H), 4.69 (s, 2H), 2.49-2.42 (m, 2H), 2.10-1.94 (m, 6H), 1.85-1.76 (m, 2H), 1.43 (s, 9H).

### Step 4

LCMS m/z 267.0 (M−H)⁻ (ES⁻). ¹H NMR (400 MHz, DMSO) δ 13.22 (s, 1H), 6.81 (d, J=2.9 Hz, 2H), 4.97-4.86 (m, 1H), 4.72 (s, 2H), 2.49-2.42 (m, 2H), 2.10-1.93 (m, 6H), 1.85-1.76 (m, 2H).

### Example 10: (E)-2-((4-(cycloheptyloxy)-4-oxobut-2-enoyl)oxy)acetic acid

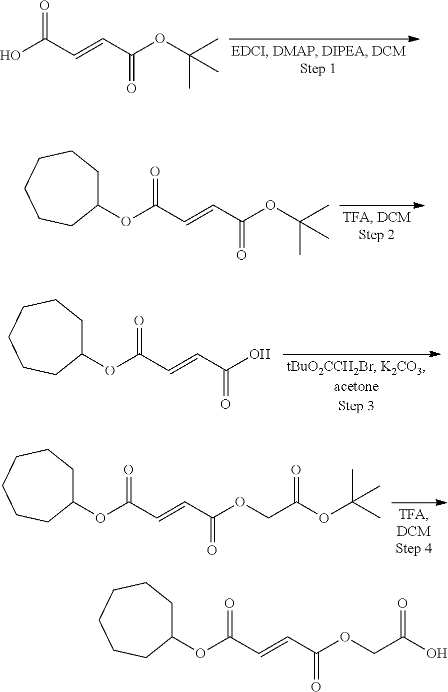 [SMILES: CC(C)(C)OC(=O)/C=C/C(=O)O ; CC(C)(C)OC(=O)/C=C/C(=O)OC1CCCCCC1 ; O=C(O)/C=C/C(=O)OC1CCCCCC1 ; CC(C)(C)OC(=O)COC(=O)/C=C/C(=O)OC1CCCCCC1 ; O=C(O)COC(=O)/C=C/C(=O)OC1CCCCCC1]

Prepared using a similar procedure to (E)-2-((4-(cyclohexyloxy)-4-oxobut-2-enoyl)oxy)acetic acid.

### Step 1

¹H NMR (400 MHz, DMSO) δ 6.64 (d, J=1.7 Hz, 2H), 4.95 (tt, J=8.0, 4.5 Hz, 1H), 1.94-1.84 (m, 2H), 1.72-1.42 (m, 19H).

### Step 2, Intermediate 8: (E)-4-(cycloheptyloxy)-4-oxobut-2-enoic acid

LCMS m/z 211.0 (M−H)⁻ (ES⁻). ¹H NMR (400 MHz, DMSO) δ 13.19 (s, 1H), 6.67 (d, J=1.2 Hz, 2H), 4.96 (tt, J=8.2, 4.4 Hz, 1H), 1.95-1.82 (m, 2H), 1.73-1.37 (m, 10H).

### Step 3

LCMS m/z 349.2 (M+Na)⁺ (ES⁺). ¹H NMR (400 MHz, DMSO) δ 6.82 (s, 2H), 4.98 (tt, J=8.3, 4.5 Hz, 1H), 4.70 (s, 2H), 1.95-1.83 (m, 2H), 1.75-1.44 (m, 10H), 1.43 (s, 9H).

### Step 4

LCMS m/z 269.0 (M−H)⁻ (ES⁻). ¹H NMR (400 MHz, DMSO) δ 13.22 (s, 1H), 6.81 (s, 2H), 4.98 (tt, J=8.2, 4.4 Hz, 1H), 4.72 (s, 2H), 1.96-1.84 (m, 2H), 1.76-1.38 (m, 10H).

### Example 11: (E)-3-((4-(cyclooctyloxy)-4-oxobut-2-enoyl)oxy)butanoic acid

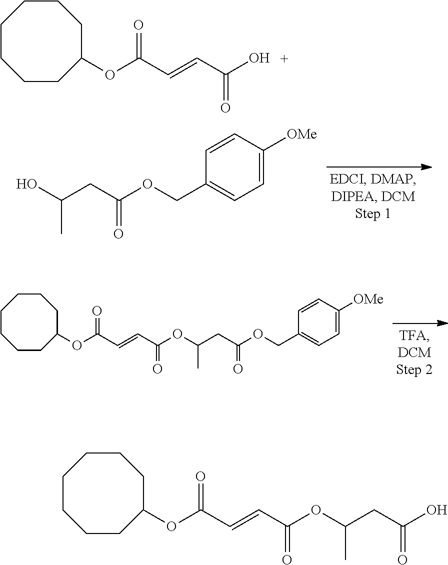

Prepared using a similar procedure to (E)-3-((4-(cyclooctyloxy)-4-oxobut-2-enoyl)oxy)-2,2-dimethylpropanoic acid using 4-methoxybenzyl 3-hydroxybutanoate.

### Step 1

LCMS m/z 454.6 (M+Na)⁺ (ES⁺). ¹H NMR (400 MHz, DMSO) δ 7.30-7.21 (m, 2H), 6.93-6.84 (m, 2H), 6.64 (d, J=15.8 Hz, 1H), 6.59 (d, J=15.8 Hz, 1H), 5.29-5.18 (m, 1H), 5.07-4.89 (m, 3H), 3.73 (s, 3H), 2.80-2.63 (m, 2H), 1.86-1.37 (m, 14H), 1.25 (d, J=6.3 Hz, 3H).

### Step 2

LCMS m/z 334.9 (M+Na)⁺ (ES⁺). ¹H NMR (400 MHz, DMSO) δ 12.38 (s, 1H), 6.71 (d, J=18.4 Hz, 1H), 6.66 (d, J=18.4 Hz, 1H), 5.21 (h, J=6.4 Hz, 1H), 4.95 (tt, J=8.2, 4.3 Hz, 1H), 2.60 (d, J=6.6 Hz, 2H), 1.86-1.38 (m, 14H), 1.26 (d, J=6.3 Hz, 3H).

### Example 12: (R,E)-2-((4-(cyclooctyloxy)-4-oxobut-2-enoyl)oxy)propanoic acid

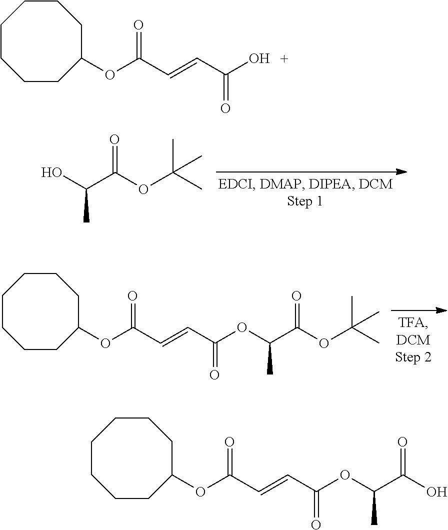

Prepared using a similar procedure to (E)-3-((4-(cyclohexyloxy)-4-oxobut-2-enoyl)oxy)propanoic acid using (R)-tert-butyl 2-hydroxypropanoate.

### Step 1

LCMS m/z 377.0 (M+Na)⁺ (ES⁺). ¹H NMR (400 MHz, DMSO) δ 6.80 (d, J=17.3 Hz, 1H), 6.76 (d, J=17.1 Hz, 1H), 5.02-4.92 (m, 2H), 1.86-1.45 (m, 14H), 1.43 (d, J=7.0 Hz, 3H), 1.40 (s, 9H).

### Step 2

LCMS m/z 321.2 (M+Na)⁺ (ES⁺). ¹H NMR (400 MHz, DMSO) δ 13.19 (s, 1H), 6.80 (d, J=16.6 Hz, 1H), 6.75 (d, J=16.5 Hz, 1H), 5.04 (q, J=7.0 Hz, 1H), 4.96 (tt, J=8.2, 4.3 Hz, 1H), 1.87-1.38 (m, 17H).

### Example 13: 2-[(E)-4-[(1R)-1-methylheptoxy]-4-oxo-but-2-enoyl]oxyacetic acid

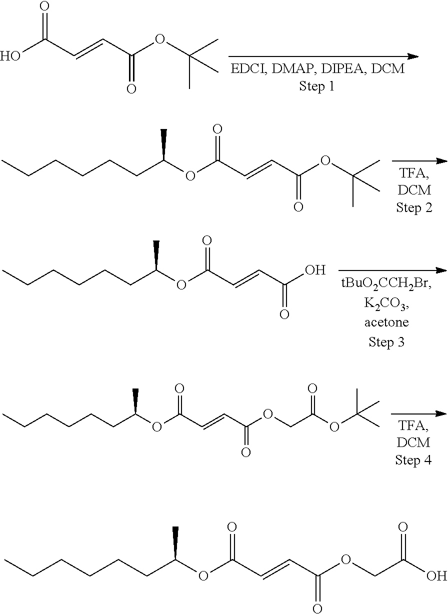

Prepared using a similar procedure to (E)-2-((4-(cyclohexyloxy)-4-oxobut-2-enoyl)oxy)acetic acid.

### Step 1

¹H NMR (400 MHz, DMSO) δ 6.64 (d, J=2.1 Hz, 2H), 4.96-4.87 (m, 1H), 1.47 (s, 9H), 1.30-1.23 (m, 10H), 1.21 (d, J=6.3 Hz, 3H), 0.89-0.83 (m, 3H).

### Step 2

LCMS m/z 227.3 (M−H)⁻ (ES⁻). ¹H NMR (400 MHz, DMSO) δ 6.67 (s 2H), 4.96-4.87 (m, 1H), 1.66-1.48 (m, 2H), 1.24 (m, 11H), 0.90-0.82 (m, 3H).

### Step 3

¹H NMR (400 MHz, DMSO) δ 6.82 (s, 2H), 4.98-4.89 (m, 1H), 4.69 (s, 2H), 1.66-1.48 (m, 2H), 1.43 (s, 9H), 1.32-1.22 (m, 11H), 0.89-0.82 (m, 3H).

### Step 4

LCMS m/z 285.0 (M−H)⁻ (ES⁻). ¹H NMR (400 MHz, DMSO) δ 13.20 (s, 1H), 6.81 (s, 2H), 5.00-4.89 (m, 1H), 4.72 (s, 2H), 1.65-1.49 (m, 2H), 1.34-1.21 (m, 11H), 0.89-0.83 (m, 3H).

### Example 14: (R,E)-3-((4-(octan-2-yloxy)-4-oxobut-2-enoyl)oxy)propanoic acid

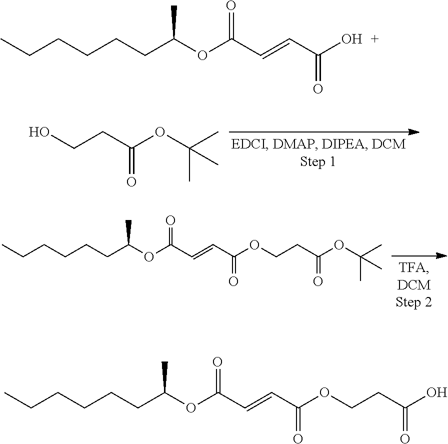

Prepared using a similar procedure to (E)-3-((4-(cyclohexyloxy)-4-oxobut-2-enoyl)oxy)propanoic acid.

### Step 1

¹H NMR (400 MHz, DMSO) δ 6.72 (s, 2H), 4.98-4.87 (m, 1H), 4.33 (t, J=6.1 Hz, 2H), 2.63 (t, J=6.1 Hz, 2H), 1.66-1.48 (m, 2H), 1.40 (s, 9H), 1.36-1.17 (m, 11H), 0.92-0.81 (m, 3H).

### Step 2

LCMS m/z 284.8 (M−H)⁻ (ES⁻). ¹H NMR (400 MHz, DMSO) δ 13.20 (s, 1H), 6.81 (s, 2H), 5.00-4.89 (m, 1H), 4.72 (s, 2H), 1.65-1.49 (m, 2H), 1.34-1.21 (m, 11H), 0.89-0.83 (m, 3H).

### Example 15: (E)-2-((4-oxo-4-(1-(4-(trifluoromethyl)phenyl)cyclobutoxy)but-2-enoyl)oxy) acetic acid

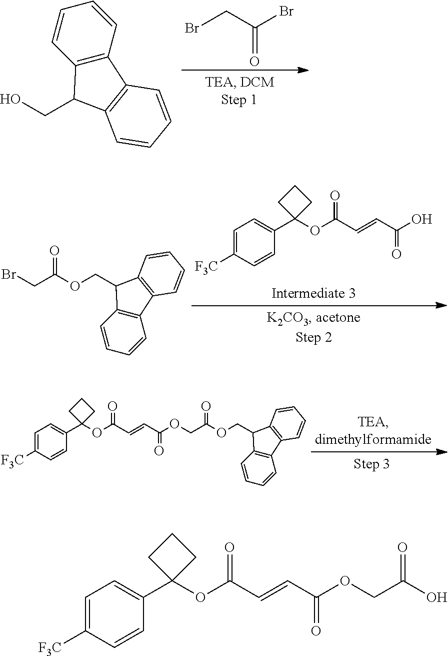

### Step 1

To the solution of (9H-fluoren-9-yl)methanol (2.0 g, 10.2 mmol) and 2-bromoacetyl bromide (4.08 g, 20.4 mmol) in DCM (40 mL) was added TEA (3.09 g, 30.6 mmol), and the mixture was stirred at room temperature for 18 h. The reaction mixture was quenched with water (40 mL), the organic layer was separated, and the aqueous layer extracted with DCM (3×40 mL). The combined organic layers were washed with brine, dried over Na₂SO₄ and filtered. The filtrate was concentrated under reduced pressure, and the residue was purified by flash column chromatography on silica (0-15% tert-butyl methyl ether/petroleum ether) to give (9H-fluoren-9-yl)methyl 2-bromoacetate (2.0 g, 62% yield) as yellow oil. LCMS m/z 339.0 (M+Na)⁺ (ES⁺).

### Step 2

A mixture of (9H-fluoren-9-yl)methyl 2-bromoacetate (310 mg, 0.98 mmol), (E)-4-oxo-4-(1-(4-(trifluoromethyl)phenyl)cyclobutoxy)but-2-enoic acid (Intermediate 3, 308 mg, 0.98 mmol) and K₂CO₃ (203 mg, 1.47 mmol) in acetone (4 mL) was stirred at room temperature for 18 h. The reaction mixture was filtered, and the filtrate was concentrated under reduced pressure. The residue was purified by flash column chromatography on silica (0-10% tert-butyl methyl ether/petroleum ether) to give 2-((9H-fluoren-9-yl)methoxy)-2-oxoethyl 1-(4-(trifluoromethyl)phenyl)cyclobutyl fumarate (250 mg, 46% yield) as a light yellow oil. LCMS m/z 572.8 (M+Na)⁺ (ES⁺).

### Step 3

A solution of 2-((9H-fluoren-9-yl)methoxy)-2-oxoethyl 1-(4-(trifluoromethyl)phenyl)cyclobutyl fumarate (250 mg, 0.45 mmol) in dimethylformamide (2 mL) and TEA (0.4 mL) was stirred at room temperature for 3 h. The reaction mixture was acidified with 0.5 N HCl until pH 5, and extracted with EtOAc (2×3 mL). The organic layer was washed by brine, dried over Na₂SO₄, filtered and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) gradient: 58-95% MeCN; collection wavelength: 214 nm). The fractions were concentrated under reduced pressure to remove MeCN, and lyophilized to give (E)-2-(4-oxo-4-(1-(4-(trifluoromethyl)phenyl)cyclobutoxy)but-2-enoyloxy)acetic acid (77.2 mg, 46% yield) as white solid. LCMS m/z 394.9 (M+Na)⁺ (ES⁺). ¹H NMR (400 MHz, DMSO) δ 13.24 (br, 1H), 7.78-7.68 (m, 4H), 6.89-7.79 (m, 2H), 4.72 (s, 2H), 2.70-2.60 (m, 4H), 2.04-1.97 (m, 1H), 1.82-1.74 (m, 1H).

### Example 16: (E)-3-(4-oxo-4-(1-(4-(trifluoromethyl)phenyl)cyclobutoxy)but-2-enoyloxy)propanoic acid

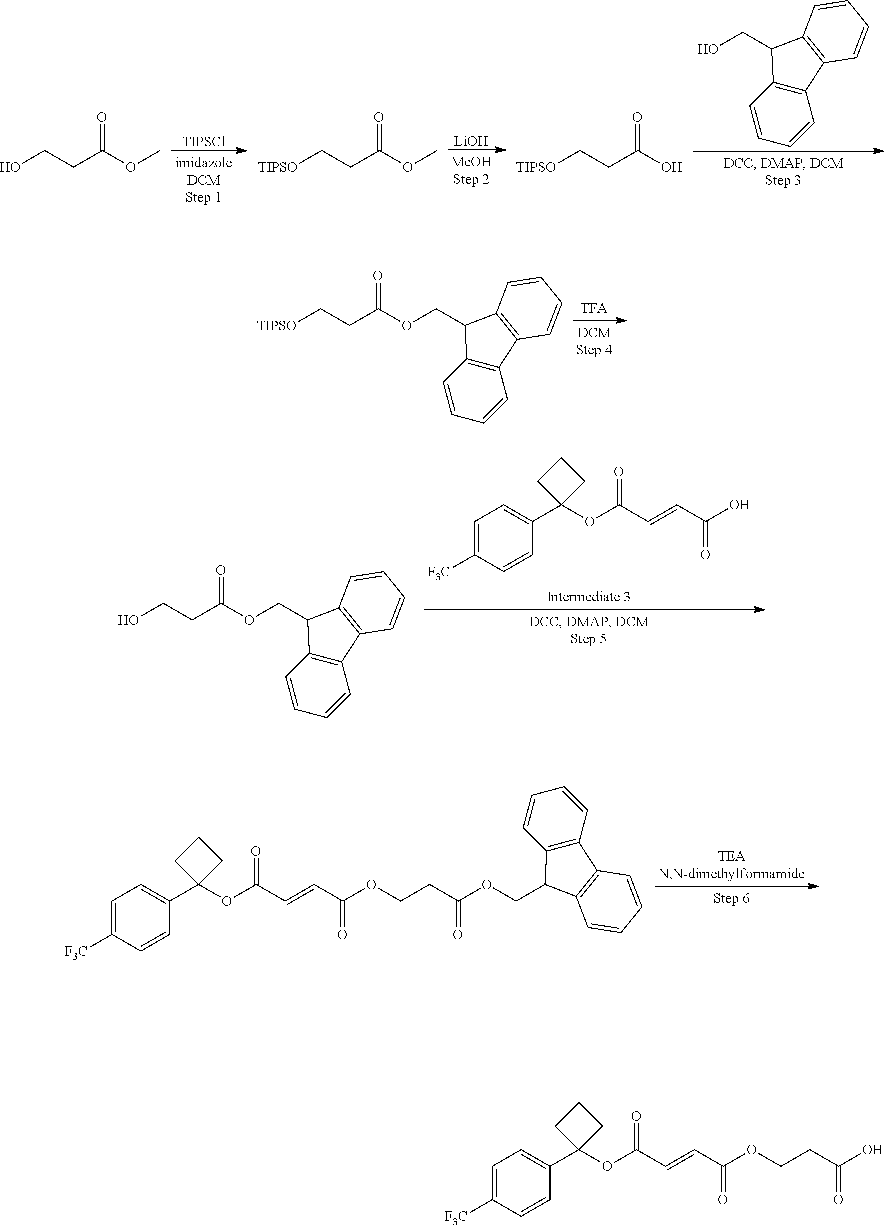

### Step 1

To the solution of methyl 3-hydroxypropanoate (SCP-29-0, 5.00 g, 48.08 mmol) and triisopropylsilyl chloride (18.60 g, 96.16 mmol) in DCM (200 mL) was added imidazole (9.80 g, 144.24 mmol), and the reaction mixture was stirred at room temperature overnight. The mixture was quenched with water (150 mL) and extracted with DCM (3×200 mL). The combined organic layers were washed with brine, dried over Na₂SO₄. and concentrated under reduced pressure. The residue was purified by flash column chromatography (0-8% tert-butyl methyl ether/petroleum ether) to give methyl 3-(triisopropylsilyloxy)propanoate (11.00 g, 88% yield) as colorless oil. ¹H NMR (400 MHz, CDCl₃) δ: 3.99 (t, J=6.4 Hz, 2H), 3.68 (s, 3H), 2.56 (t, J=6.4 Hz, 2H), 1.11-1.03 (m, 21H).

### Step 2

To a solution of methyl 3-(triisopropylsilyloxy)propanoate (11.00 g, 42.31 mmol) in MeOH (150 mL) was added 2N LiOH aqueous solution (23.27 mL, 46.54 mmol), and the reaction mixture was stirred at room temperature for 4 h. The mixture was concentrated under reduced pressure to give the residue, which was quenched with water (100 ml), extracted with MTBE (2×150 ml). The MTBE layer was washed by brine, dried over Na₂SO₄, and concentrated under reduced pressure to give 3-(triisopropylsilyloxy)propanoic acid (10.3 g, 99% yield) as light yellow oil, which was used in the next step directly. ¹H NMR (400 MHz, CDCl₃) δ: 4.00 (t, J=6.4 Hz, 2H), 2.60 (t, J=6.4 Hz, 2H), 1.14-1.03 (m, 21H). A mixture of 3-(triisopropylsilyloxy)propanoic acid (10.3 g, 41.87 mmol), (9H-fluoren-9-yl)methanol (8.21 g, 41.87 mmol), DCC (12.94 g, 62.805 mmol) and DMAP (511 mg, 4.187 mmol) in DCM (150 mL) was stirred at room temperature for 3 h. The mixture was filtered, and the filtrate was concentrated under reduced pressure. The residue was purified by flash column chromatography (0-5% tert-butyl methyl ether/petroleum ether) to give (9H-fluoren-9-yl)methyl 3-(triisopropylsilyloxy)propanoate (17 g, 95% yield) as a light yellow oil. LCMS m/z 447.0 (M+Na)⁺ (ES⁺).

### Step 3

A mixture of (9H-fluoren-9-yl)methyl 3-(triisopropylsilyloxy)propanoate (1.3 g, 3.07 mmol) in DCM (4.5 mL) and TFA (1.5 mL) was stirred at room temperature for 2 h. The reaction mixture was concentrated under reduced pressure, and the residue was purified by flash column chromatography (0-20% tert-butyl methyl ether/petroleum ether) to give (9H-fluoren-9-yl)methyl 3-hydroxypropanoate (240 mg, 29% yield) as white solid. ¹H NMR (400 MHz, CDCl₃) δ: 7.78 (d, J=7.6 Hz, 2H), 7.59 (d, J=7.2 Hz, 2H), 7.42 (t, J=7.2 Hz, 2H), 7.33 (t, J=6.4 Hz, 2H), 4.48 (d, J=6.8 Hz, 2H), 4.24 (t, J=6.4 Hz, 1H), 3.83 (t, J=6 Hz, 2H), 2.63 (t, J=5.6 Hz, 2H).

### Step 4

A mixture of (9H-fluoren-9-yl)methyl 3-hydroxypropanoate (240 mg, 0.89 mmol), (E)-4-oxo-4-(1-(4-(trifluoromethyl)phenyl)cyclobutoxy)but-2-enoic acid (Intermediate 3, 281 mg, 0.89 mmol), DCC (275 mg, 1.335 mmol) and DMAP (11 mg, 0.09 mmol) in DCM (3 mL) was stirred at room temperature overnight. The the mixture was filtered, and the filtrate was concentrated under reduced pressure. The residue was purified by flash column chromatography (0-10% tert-butyl methyl ether/petroleum ether) to give 3-((9H-fluoren-9-yl)methoxy)-3-oxopropyl 1-(4-(trifluoromethyl)phenyl)cyclobutyl fumarate (300 mg, 59% yield) as a light yellow oil. LCMS m/z 586.8 (M+Na)⁺ (ES⁺).

### Step 5

A mixture of 3-((9H-fluoren-9-yl)methoxy)-3-oxopropyl 1-(4-(trifluoromethyl)phenyl)cyclobutyl fumarate (300 mg, 0.53 mmol) in N,N-Dimethylformamide (2 mL) and triethylamine (0.4 mL) was stirred at 20° C. for 3 h. The reaction mixture was acidified with 0.5N HCl until pH=5, and extracted with EtOAc (3×3 mL). The EtOAc layer was washed by brine, dried over Na₂SO₄ and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water): MeCN gradient: 58-95%; collection wavelength: 214 nm). The fractions were concentrated under reduced pressure to remove MeCN, and lyophilized to give (E)-3-(4-oxo-4-(1-(4-(trifluoromethyl)phenyl)cyclobutoxy)but-2-enoyloxy)propanoic acid (108.21 mg, 52% yield) as light yellow oil. LCMS m/z 394.9 (M+Na)⁺ (ES⁺). ¹H NMR (400 MHz, DMSO-d₆) δ: 12.45 (br s, 1H), 7.74 (d, J=8.4 Hz, 2H), 7.70 (d, J=8.4 Hz, 2H), 7.79-7.68 (m, 2H), 4.33 (t, J=6 Hz, 2H), 2.66-2.62 (m, 6H), 2.01-1.98 (m, 1H), 1.81-1.73 (m, 1H).

### Biological Example 1—THP-1 AlphaLISA IL-1β and IL-6 Cytokine Assay

Measuring Inhibitory Effects on IL-1, and IL-6 Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were each run in a background of differentiated THP-1 cells as described below. All reagents described were from Sigma-Aldrich unless specified otherwise. Compounds were prepared as 10 mM DMSO stocks.

Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1β and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalizing the sample data to the high and low controls used within each plate (+/−LPS respectively). Percentage inhibition was then plotted against compound concentration and the 50% inhibitory concentration (IC₅₀) was determined from the resultant concentration-response curve.

The data for all compounds of formula (I) tested in this assay are presented in Table 1 below. Dimethyl fumarate and 2-(2,5-dioxopyrrolidin-1-yl)ethyl methyl fumarate (diroximel fumarate) were included as comparator compounds.

TABLE 1
THP-1 cell IL-1β and IL-6 IC₅₀ values (μM) (++++
indicates IC₅₀ of <2.5 μM; +++ indicates IC₅₀ of 2.5
to 6.1 μM; ++ indicates IC₅₀ of 6.2 to 9.2 μM; +
indicates IC₅₀ of 9.3 to 14.3 μM)

| Compound | IL-1β (IC₅₀) | IL-6 (IC₅₀) |
|---|---|---|
| dimethyl fumarate | 14.4 | 9.3 |
| 2-(2,5-dioxopyrrolidin-1- | 6.2 | NT* |
| yl)ethyl methyl fumarate | | |
| Example 1 | +++ | ++++ |
| Example 2 | + | ++ |
| Example 3 | ++++ | ++++ |
| Example 4 | +++ | ++++ |
| | | +++^{a} |
| Example 5 | +++ | NT |
| Example 6 | ++++ | NT |
| Example 7 | ++++ | NT |
| Example 8 | ++++ | NT |
| Example 9 | +++ | NT |
| Example 10 | ++++ | NT |
| Example 11 | ++++ | NT |
| Example 12 | ++++ | NT |
| Example 13 | +++ | NT |
| Example 14 | ++++ | NT |
| Example 15 | ++++ | +++ |
| Example 16 | +++ | ++++ |

NT* = not tested;
^{a}from repeated experiments

These results reveal that compounds of formula (I) are expected to have anti-inflammatory activity as shown by their IC₅₀ values for inhibition of IL-1β and IL-6 release in this assay. All compounds of the invention tested exhibited improved IL-1β and IL-6 lowering properties (IC₅₀ values) compared to dimethyl fumarate. Certain compounds of the invention tested exhibited improved IL-1β lowering properties (IC₅₀ values) compared to 2-(2,5-dioxopyrrolidin-1-yl)ethyl methyl fumarate (diroximel fumarate).

The data for all compounds of formula (II) tested in this assay are presented in Table 2 below. Monomethyl fumarate was included as a comparator compound.

TABLE 2
THP-1 cell IL-1β and IL-6 IC₅₀ values (μM) (++++
indicates IC₅₀ of <6 μM, +++ indicates IC₅₀ of 6-
30 μM, ++ indicates IC₅₀ of >30-50 μM; + indicates
IC₅₀ of >50-100 μM)

| Compound | IL-1β (IC₅₀) | IL-6 (IC₅₀) |
|---|---|---|
| monomethyl fumarate | >100 | >100 |
| Intermediate 3 | ++++ | ++++ |
| Intermediate 4 | >100 | NT |
| Intermediate 6 | +++ | NT* |
| Intermediate 7 | +++ | NT |
| Intermediate 8 | ++ | NT |
| Intermediate 9 | +++ | +++ |
| Intermediate 10 | +++ | +++ |
| Intermediate 11 | + | >100 |
| Intermediate 12 | +++ | +++ |
| Intermediate 13 | +++ | +++ |
| Intermediate 14 | +++ | ++++ |
| Intermediate 15 | ++ | >100 |
| Intermediate 16 | ++++ | ++++ |
| Intermediate 17 | +++ | +++ |
| Intermediate 18 | +++ | +++ |
| Intermediate 19 | +++ | +++ |
| Intermediate 20 | +++ | +++ |
| Intermediate 21 | +++ | >100 |
| Intermediate 23 | +++ | +++ |
| Intermediate 24 | ++ | +++ |
| Intermediate 25 | +++ | ++++ |
| monomethyl fumarate | >100 | >100 |
| Intermediate 26 | ++++ | ++++ |
| Intermediate 27 | + | +++ |
| Intermediate 28 | +++ | +++ |
| Intermediate 29 | ++++ | ++++ |
| Intermediate 30 | ++++ | +++ |
| Intermediate 31 | +++ | +++ |
| Intermediate 32 | ++++ | ++++ |
| Intermediate 33 | ++++ | ++++ |
| Intermediate 34 | +++ | ++ |
| Intermediate 35 | + | >100 |
| Intermediate 36 | ++++ | NT |
| Intermediate 38 | ++++ | +++ |
| Intermediate 39 | ++ | ++ |
| Intermediate 40 | >100 | ++ |
| Intermediate 41 | +++ | +++ |
| Intermediate 44 | ++++ | ++++ |
| Intermediate 45 | ++++ | ++++ |

*NT means not tested

The compounds of formula (II) shown in Table 2 exhibited improved cytokine-lowering potencies compared to monomethyl fumarate, as shown by the lower IL-1β and/or IL-6 IC₅₀ values (where tested), and thus are expected to exhibit anti-inflammatory activity. Intermediate 4 was not active in these assays. Preferred compounds of formula (II) are also more potent than dimethyl fumarate and 2-(2,5-dioxopyrrolidin-1-yl)ethyl methyl fumarate, the values for which are shown in Table 1.

### Biological Example 2—NRF2 Activation Assay

Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

Additionally, a defined concentration of dimethyl fumarate was used as the ‘High’ control to normalise test compound activation responses to.

Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound.

Following compound treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

The data for all compounds of formula (I) tested in this assay are presented in Table 3 below. Dimethyl fumarate was included as the comparator compound.

TABLE 3
NRF2 activation (For EC₅₀ values, ++++ means <2.0, +++ means
2.1-6.0 μM, ++ means >6.0-20 μM and + means >20-100 μM; for
Eₘₐₓ values ++++ means 200-250%, +++ means
150-199%, ++ means 100-149 and + means >17.1-99)

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl fumarate | 6.1 | 100 | >100 | 17.1 |
| Example 1 | ++++ | ++++ | ++ | ++ |
| Example 2 | +++ | ++++ | + | + |
| dimethyl fumarate | 6.1 | 100 | >100 | 17.1 |
| Example 3 | ++++ | ++++ | + | ++ |
| Example 4 | +++ | +++ | ++ | + |
| Example 5 | +++ | +++ | + | + |
| Example 6 | ++++ | ++++ | ++ | + |
| Example 7 | ++++ | ++++ | ++ | + |
| Example 8 | ++++ | ++++ | + | ++ |
| Example 11 | ++++ | ++++ | + | + |
| Example 12 | ++++ | ++++ | ++ | ++ |
| Example 13 | ++++ | +++ | + | ++ |
| Example 14 | +++ | +++ | ++ | ++ |
| Example 15 | +++ | +++ | >100 | + |

NT means not tested

These results reveal that compounds of formula (I) are expected to have anti-inflammatory activity as shown by their EC₅₀ and Eₘₐₓ values for NRF2 activation in this assay. All Examples tested exhibited higher potency, as shown by lower EC₅₀ values and higher Eₘₐₓ in −GSH and/or +GSH assays, compared to dimethyl fumarate.

The data for all compounds of formula (II) tested in this assay are presented in Table 4 below. Monomethyl fumarate was included as the comparator compound.

TABLE 4
NRF2 activation (For EC₅₀ values, +++ means <23.0
uM, ++ means 23.0-<48.4 μM; for Eₘₐₓ values ++++
means >150%, +++ means >114-150%, ++
means >87-114%, + means >50-<87%)

| | −GSH | | +GSH | |
|---|---|---|---|---|
| Compound | EC₅₀ (μM) | Eₘₐₓ (%) | EC₅₀ (μM) | Eₘₐₓ (%) |
| monomethyl | 23.0 | 114 | 48.4 | 87 |
| fumarate | | | | |
| Intermediate 3 | +++ | +++ | +++ | +++ |
| Intermediate 4 | +++ | +++ | ++ | + |
| Intermediate 5 | +++ | ++++ | +++ | ++++ |
| Intermediate 6 | +++ | ++++ | +++ | ++++ |
| Intermediate 7 | +++ | ++++ | +++ | +++ |
| monomethyl | 23.0 | 114 | 48.4 | 87 |
| fumarate | | | | |
| Intermediate 8 | +++ | ++++ | ++ | +++ |
| Intermediate 10 | +++ | +++ | +++ | +++ |
| Intermediate 12 | +++ | ++ | +++ | ++ |
| Intermediate 14 | +++ | ++++ | +++ | ++++ |
| Intermediate 16 | +++ | ++++ | +++ | +++ |
| Intermediate 19 | +++ | ++++ | +++ | +++ |
| Intermediate 20 | +++ | +++ | ++ | + |
| Intermediate 21 | +++ | +++ | +++ | ++ |
| | | | ++^{a} | |
| Intermediate 25 | +++ | ++++ | +++ | ++ |
| Intermediate 26 | +++ | +++ | +++ | ++ |
| Intermediate 30 | +++ | +++ | ++ | ++ |
| Intermediate 36 | +++ | +++ | +++ | ++ |
| Intermediate 38 | +++ | ++++ | +++ | + |

^{a}from repeated experiments

These results reveal that compounds of formula (II) are expected to have anti-inflammatory activity as shown by their EC₅₀ and Eₘₐₓ values for NRF2 activation in this assay. All compounds tested exhibited higher potency, as shown by lower EC₅₀ values and/or higher Eₘₐₓ values in the −GSH and/or +GSH assay, compared to dimethyl fumarate. In addition, the activities of the compounds of formula (II) shown in Table 4 were much less susceptible to the effects of added GSH. Preferred compounds have +++EC₅₀ values and at least +++Eₘₐₓ values in the −GSH and +GSH assays.

### Biological Example 3—Hepatocyte Stability Assay

Defrosted cryo-preserved hepatocytes (viability >70%) are used to determine the metabolic stability of a compound via calculation of intrinsic clearance (CIᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involves a time-dependent reaction using both positive and negative controls. The cells must be pre-incubated at 37° C. then spiked with test compound (and positive control); samples taken at pre-determined time intervals are analysed to monitor the change in concentration of the initial drug compound over 60 minutes. A buffer incubation reaction (with no hepatocytes present) acts as a negative control and two cocktail solutions, containing compounds with known high and low clearance values (verapamil/7-hydroxycoumarin and propranolol/diltiazem), act as positive controls.
1. The assay is run with a cell concentration of 0.5×10⁶ cells/mL in Leibovitz buffer.
2. All compounds and controls are run in duplicate.
3. Compound concentration is 10 μM.
4. All compounds and controls are incubated with both cells and buffer to show turnover is due to hepatic metabolism.
5. All wells on the incubation plate will have 326.7 μL of either cells or buffer added.
6. Prior to assay, cell and buffer-only incubation plates are preincubated for 10 mins at 37° C.
7. The assay is initiated by adding compounds, 3.3 μL of 1 mM in 10% DMSO-90% Buffer; final DMSO concentration is 0.1%.
8. Samples are taken at regular timepoints (0, 5, 10, 20, 40, 60 min) until 60 mins.
9. Sample volume is 40 μL and it is added to 160 μL of crash solvent (acetonitrile with internal standard) and stored on ice.
10. At the end of the assay, the crash plates are centrifuged at 3500 rpm for 20 mins at 4° C.
11. 80 μL of clear supernatant is removed and mixed with 80 μL of deionised water before being analysed by LC-MS/MS.

Raw LC-MS/MS data are exported to, and analysed in, Microsoft Excel for determination of intrinsic clearance. The percentage remaining of a compound is monitored using the peak area of the initial concentration as 100%. Intrinsic clearance and half-life values are calculated using a graph of the natural log of percentage remaining versus the time of reaction in minutes. Half-life (min) and intrinsic clearance (CIᵢₙₜ in μL min⁻¹ 10⁻⁶ cells) values are calculated using the gradient of the graph (the elimination rate constant, k) and Equations 1 and 2.

$${t}_{\frac{1}{2}} = \frac{ln 2}{k} \qquad \{ Equation 1 \}$$

$${Cl}_{int} = ( \frac{ln 2}{{t}_{\frac{1}{2}}} ) \times ( \frac{350}{0.175} ) \qquad \{ Equation 2 \}$$

The data for all compounds of formula (I) tested in this assay are presented in Table 5 below. 2-(2,5-Dioxopyrrolidin-1-yl)ethyl methyl fumarate (diroximel fumarate) was included as the comparator compound.

TABLE 5
Hepatocyte stability (For CIᵢₙₜ (μL min⁻¹ 10⁻⁶ cells; mouse), ++ means
200-277 and + means >277; For CIᵢₙₜ (μL min⁻¹ 10⁻⁶ cells; human), ++++
means <50, +++ means 50-149, ++ means 150-277, and + means >277;
For T½ (min; mouse), ++ means 5-10, and + means <5;
For T½ (min; human), ++++ means >100, +++
means 30-100, ++ means 5-29, and + means <5)

| | | CIᵢₙₜ | T½ |
|---|---|---|---|
| Compound | Species | (μL min⁻¹ 10⁻⁶ cells) | (min) |
| 2-(2,5-dioxopyrrolidin-1- | Human | >277 | <5 |
| yl)ethyl methyl fumarate | Mouse | >277 | <5 |
| Example 1 | Human | ++++ | +++ |
| | Mouse | + | + |
| Example 2 | Human | ++++ | +++ |
| | Mouse | ++ | ++ |
| Example 3 | Human | +++ | ++ |
| | Mouse | + | + |
| Example 4 | Human | +++ | ++ |
| | Mouse | + | + |
| Example 5 | Human | ++ | ++ |
| | Mouse | + | + |
| Example 6 | Human | ++++ | +++ |
| | Mouse | ++ | ++ |
| Example 7 | Human | +++ | ++ |
| | Mouse | + | + |
| Example 8 | Human | +++ | ++ |
| | Mouse | + | + |
| Example 9 | Human | ++++ | +++ |
| | Mouse | + | + |
| Example 10 | Human | ++++ | +++ |
| | Mouse | + | + |
| Example 11 | Human | +++ | ++ |
| | Mouse | + | + |
| Example 12 | Human | ++++ | ++++ |
| | Mouse | + | + |
| Example 13 | Human | +++ | ++ |
| | Mouse | + | + |
| 2-(2,5-dioxopyrrolidin-1- | Human | >277 | <5 |
| yl)ethyl methyl fumarate | Mouse | >277 | <5 |
| Example 14 | Human | + | + |
| | Mouse | + | + |
| Example 15 | Human | +++ | ++ |
| | Mouse | + | + |
| Example 16 | Human | +++ | ++ |
| | Mouse | + | + |

These results reveal that compounds of the invention are expected to have improved metabolic stabilities, as shown by their intrinsic clearance (CIᵢₙₜ) and half-life (T_{1/2}) values in this assay. All the compounds of formula (I), except Example 14, shown in Table 5 were more stable, i.e., they exhibited lower intrinsic clearance (CIᵢₙₜ) and longer half-life (T_{1/2}) values compared with 2-(2,5-dioxopyrrolidin-1-yl)ethyl methyl fumarate (diroximel fumarate) in at least human hepatocytes. Preferred compounds exhibited lower intrinsic clearance (CIᵢₙₜ) and longer half-life (T_{1/2}) values compared with 2-(2,5-dioxopyrrolidin-1-yl)ethyl methyl fumarate (diroximel fumarate) in both human and mouse species.

TABLE 6
Hepatocyte stability (For CIᵢₙₜ (μL min⁻¹ 10⁻⁶ cells), ++++
means 5-15, +++ means >15-30, ++ means >30-110, +
means >110-277 and + means >277; For T½ (min), ++++
means >100, +++ means 30-100, ++ means 5-29, and + means <5)

| | | CIᵢₙₜ | T½ |
|---|---|---|---|
| Compound | Species | (HL min⁻¹ 10⁻⁶ cells) | (min) |
| 2-(2,5-dioxopyrrolidin-1- | Human | >277 | <5 |
| yl)ethyl methyl fumarate | Mouse | >277 | <5 |
| Intermediate 3 | Human | ++++ | ++++ |
| | Mouse | +++ | +++ |
| Intermediate 6 | Human | ++++ | ++++ |
| | Mouse | ++ | ++ |
| Intermediate 7 | Human | +++ | +++ |
| | Mouse | ++ | ++ |
| Intermediate 8 | Human | +++ | +++ |
| | Mouse | ++ | +++ |
| Intermediate 10 | Human | ++ | ++ |
| | Mouse | ++ | +++ |
| Intermediate 12 | Human | +++ | +++ |
| | Mouse | +++ | +++ |
| 2-(2,5-dioxopyrrolidin-1- | Human | >277 | <5 |
| yl)ethyl methyl fumarate | Mouse | >277 | <5 |
| Intermediate 14 | Human | ++++ | ++++ |
| | Mouse | ++ | ++ |
| Intermediate 16 | Human | +++ | ++++ |
| | Mouse | +++ | +++ |
| Intermediate 17 | Human | ++ | ++ |
| | Mouse | + | ++ |
| Intermediate 18 | Human | ++ | +++ |
| | Mouse | ++ | +++ |
| Intermediate 19 | Human | ++++ | ++++ |
| | Mouse | ++ | ++ |
| Intermediate 20 | Human | ++++ | ++++ |
| | Mouse | +++ | +++ |
| Intermediate 21 | Human | ++++ | ++++ |
| | Mouse | ++++ | ++++ |
| Intermediate 25 | Human | +++ | ++++ |
| | Mouse | ++ | +++ |
| Intermediate 26 | Human | +++ | +++ |
| | Mouse | ++ | +++ |
| Intermediate 29 | Human | ++++ | ++++ |
| | Mouse | +++ | +++ |
| Intermediate 30 | Human | ++++ | ++++ |
| | Mouse | +++ | +++ |
| Intermediate 32 | Human | +++ | +++ |
| | Mouse | +++ | +++ |
| Intermediate 33 | Human | ++++ | ++++ |
| | Mouse | +++ | +++ |
| Intermediate 36 | Human | ++++ | ++++ |
| | Mouse | ++ | +++ |
| Intermediate 38 | Human | +++ | +++ |
| | Mouse | ++ | ++ |
| Intermediate 41 | Human | ++++ | ++++ |
| | Mouse | ++ | +++ |
| 2-(2,5-dioxopyrrolidin-1- | Human | >277 | <5 |
| yl)ethyl methyl fumarate | Mouse | >277 | <5 |
| Intermediate 44 | Human | ++ | ++ |
| | Mouse | + | + |
| Intermediate 45 | Human | ++ | ++ |
| | Mouse | + | 4 |

All the compounds of formula (II) shown in Table 6 were more stable, i.e., they exhibited lower intrinsic clearance (CIᵢₙₜ) and longer half-life (T_{1/2}) values compared with 2-(2,5-dioxopyrrolidin-1-yl)ethyl methyl fumarate (diroximel fumarate) in at least human hepatocytes.

### REFERENCES

The following publications cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
Brück J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. medRxiv 2019, 19001594; doi: https://doi.org/10.1101/19001594.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020 doi:10.1111/imcb.12316.
Hanke T. et al. Pharmacol. Therapeut. 2016, 157, 163-187.
Hooftman A. & O'Neill L. A. J. Trends in Immunology 2019, 40, 687-698.
Hunt T. et al. Consortium of Multiple Sclerosis Centers 2015 Annual Meeting, 27-30 May 2015, Indianapolis, IN, USA: Poster DX37.
Kornberg M. D. et al. Science 2018, 360, 449-453.
Kulkarni R. A. et al. Nat. Chem. Biol. 2019 10.1038/s41589-018-0217-y.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
Naismith R. T. et al., CNS Drugs 2020, 34, 185-196
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhao C. et al. Microb. Pathogen. 2019, 133, 103541.
Zhao G. et al. Biochem. Biophys. Res. Commun. 2014, 448, 303-307.

### Miscellaneous

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1: A compound of formula (I): 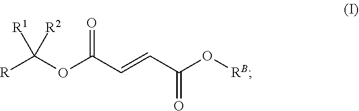
wherein:
R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₄₋₁₀ cycloalkyl ring, wherein the C₄₋₁₀ cycloalkyl ring is optionally substituted by one or more R^{c} wherein R^{c} is independently selected from the group consisting of halo, C₁₋₂ alkyl, C₁₋₂ haloalkyl, C₁₋₂ alkoxy and C₁₋₂ haloalkoxy, and/or wherein the C₄₋₁₀ cycloalkyl ring is optionally substituted by two R^{c} groups wherein the two R^{C} groups are attached to the same carbon atom and are joined to form a C₄₋₆ cycloalkyl ring; and
R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl, and/or wherein R^{B} is optionally substituted by two R^{B′} groups, attached to the same carbon atom, that are joined to form a C₃₋₆ cycloalkyl or a 4-6-membered heterocyclic ring;
wherein the total number of carbon atoms in groups R, R¹ and R² taken together, including their optional substituents, and including the carbon to which R, R¹ and R² are attached, is 6 to 14;
or a pharmaceutically acceptable salt and/or solvate thereof.

2. (canceled)

3: The compound or a pharmaceutically acceptable salt and/or solvate thereof according to claim 1 wherein R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring.

4-17. (canceled)

18: The compound or a pharmaceutically acceptable salt and/or solvate thereof according to claim 1 wherein R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano.

19-49. (canceled)

50: The compound or a pharmaceutically acceptable salt and/or solvate thereof according to claim 1 wherein R is H, methyl or CF₃ and R¹ and R² are joined to form a C₄₋₁₀ cycloalkyl ring.

51-65. (canceled)

66: The compound or a pharmaceutically acceptable salt and/or solvate thereof according to claim 1 wherein R^{B} is CH₂COOH.

67: The compound or a pharmaceutically acceptable salt and/or solvate thereof according to claim 1 wherein R^{B} is CH₂CH₂COOH.

68: The compound or a pharmaceutically acceptable salt and/or solvate thereof according to claim 1 wherein R^{B} is CH₂tetrazolyl.

69: The compound or a pharmaceutically acceptable salt and/or solvate thereof according to claim 1 wherein R^{B} is CH₂CH₂tetrazolyl.

70-77. (canceled)

78: The compound according to claim 1 which is a compound selected from the group consisting of:
(E)-2-((4-(cyclooctyloxy)-4-oxobut-2-enoyl)oxy)acetic acid;
(E)-2-((4-(cyclohexyloxy)-4-oxobut-2-enoyl)oxy)acetic acid;
(E)-3-((4-(Cyclooctyloxy)-4-oxobut-2-enoyl)oxy)propanoic acid;
(E)-3-((4-(cyclohexyloxy)-4-oxobut-2-enoyl)oxy)propanoic acid;
2-(1H-tetrazol-5-yl)ethyl cyclooctyl fumarate;
(S,E)-2-((4-(cyclooctyloxy)-4-oxobut-2-enoyl)oxy)propanoic acid;
(E)-3-((4-(cyclooctyloxy)-4-oxobut-2-enoyl)oxy)-2,2-dimethylpropanoic acid;
(E)-1-((4-(cyclooctyloxy)-4-oxobut-2-enoyl)oxy)cyclopropane-1-carboxylic acid;
(E)-2-((4-oxo-4-(spiro[3.3]heptan-2-yloxy)but-2-enoyl)oxy)acetic acid;
(E)-2-((4-(cycloheptyloxy)-4-oxobut-2-enoyl)oxy)acetic acid;
(E)-3-((4-(cyclooctyloxy)-4-oxobut-2-enoyl)oxy)butanoic acid;
(R,E)-2-((4-(cyclooctyloxy)-4-oxobut-2-enoyl)oxy)propanoic acid;
(E)-2-((4-(cycloheptyloxy)-4-oxobut-2-enoyl)oxy)acetic acid;
(R,E)-3-((4-(octan-2-yloxy)-4-oxobut-2-enoyl)oxy)propanoic acid;
(E)-2-((4-oxo-4-(1-(4-(trifluoromethyl)phenyl)cyclobutoxy)but-2-enoyl)oxy) acetic acid; and
(E)-3-(4-oxo-4-(1-(4-(trifluoromethyl)phenyl)cyclobutoxy)but-2-enoyloxy)propanoic acid;
or a pharmaceutically acceptable salt and/or solvate of any one thereof.

79: A pharmaceutical composition comprising the compound of formula (I) or a pharmaceutically acceptable salt and/or solvate thereof according to claim 1, and one or more pharmaceutically acceptable diluents or carriers.

80-82. (canceled)

83: A method of treating or preventing an inflammatory disease or a disease associated with an undesirable immune response, which comprises the step of administering the compound or a pharmaceutically acceptable salt and/or solvate thereof according to claim 1.

84-87. (canceled)

88: The method according to claim 83, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease, autoimmune paraneoplastic retinopathy, transplantation rejection (including antibody-mediated and T cell-mediated forms), multiple sclerosis, transverse myelitis, ischaemia-reperfusion injury, AGE-induced genome damage, an inflammatory bowel disease, primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders, and renal inflammatory disorders.

89-95. (canceled)

96: The method according to claim 83 wherein the compound or pharmaceutically acceptable salt and/or solvate thereof is administered in combination with a further therapeutic agent selected from the group consisting of a corticosteroid (glucocorticoid), retinoid, anthralin, vitamin D analogue, calcineurin inhibitors, phototherapy or photochemotherapy or other form of ultraviolet light irradiation therapy, ciclosporine, a thiopurine, methotrexate, an anti-TNFα agents, phosphodiesterase-4 (PDE4) inhibition, anti-IL-17 agent, anti-IL12/IL-23 agent, anti-IL-23 agent, JAK (Janus Kinase) inhibitor, plasma exchange, intravenous immune globulin (IVIG), cyclophosphamide, anti-CD20 B cell depleting agent, anthracycline analogue, cladribine, sphingosine 1-phosphate receptor modulator or sphingosine analogue, interferon beta preparation (including interferon beta 1b/1a), glatiramer, anti-CD3 therapy, anti-CD52 targeting agent, leflunomide, teriflunomide, gold compound, laquinimod, potassium channel blocker, mycophenolic acid, mycophenolate mofetil, purine analogue, mTOR (mechanistic target of rapamycin) pathway inhibitor, anti-thymocyte globulin (ATG), IL-2 receptor (CD25) inhibitor, anti-IL-6 receptor or anti-IL-6 agent, Bruton's tyrosine kinase (BTK) inhibitor, tyrosine kinase inhibitor, ursodeoxycholic acid, hydroxychloroquine, chloroquine, B cell activating factor (BAFF, also known as BLyS, B lymphocyte stimulator) inhibitor, other B cell targeted therapy including a fusion protein targeting both APRIL (A PRoliferation-Inducing Ligand) and BLyS, PI3K inhibitor including pan-inhibitor or one targeting the p110δ and/or p110γ containing isoforms, an interferon α receptor inhibitor, T cell co-stimulation blocker, thalidomide and its derivatives, dapsone, clofazimine, a leukotriene antagonist, theophylline, anti-IgE therapy, an anti-IL-5 agent, a long-acting muscarinic agent, a PDE4 inhibitor, riluzole, a free radical scavenger, a proteasome inhibitor, a complement cascade inhibitor including one directed against C5, immunoadsor, antithymocyte globulin, 5-aminosalicylates and their derivatives, an anti-integrin agent including one targeting α4β1 and/or α4β7 integrins, an anti-CD11-α agent, a non-steroidal anti-inflammatory drug (NSAID) including a salicylate, a propionic acid, an acetic, an oxicam, a fenamate, a selective or relatively selective COX-2 inhibitor, colchicine, an IL-4 receptor inhibitor, topical/contact immunotherapy, anti-IL-1 receptor therapy, IL-1β inhibitor, IL-1 neutralising therapy, chlorambucil, a specific antibiotic with immunomodulatory properties and/or ability to modulate NRF2, anti-androgenic therapy, pentoxifylline, ursodeoxycholic acid, obeticholic acid, fibrate, a cystic fibrosis transmembrane conductance regulator (CFTR) modulator, a VEGF (vascular endothelial growth factor) inhibitor, pirfenidone and mizoribine.

97: A compound of formula (a) (I-P): 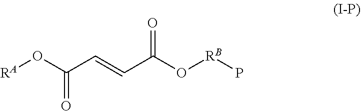
or a salt thereof, wherein R^{A} is 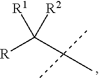
and R^{B} is defined in claim 1 and P is a carboxylic acid protecting group; or
(b)(III) 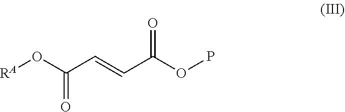
or a salt thereof, wherein R^{A} is 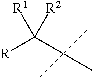
and P is a carboxylic acid protecting group;
wherein R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₄₋₁₀ cycloalkyl ring, wherein the C₄₋₁₀ cycloalkyl ring is optionally substituted by one or more R^{c} wherein R^{c} is independently selected from the group consisting of halo, C₁₋₂ alkyl, C₁₋₂ haloalkyl, C₁₋₂ alkoxy and C₁₋₂ haloalkoxy, and/or wherein the C₄₋₁₀ cycloalkyl ring is optionally substituted by two R^{c} groups wherein the two R^{C} groups are attached to the same carbon atom and are joined to form a C₄₋₆ cycloalkyl ring.

98: A compound of formula (II): 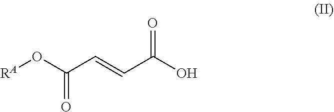
or a salt thereof, wherein R^{A} is defined in claim 1.

99-100. (canceled)

101: A compound selected from the group consisting of:
2-(tert-butoxy)-2-oxoethyl cyclooctyl fumarate;
2-(tert-butoxy)-2-oxoethyl cyclohexyl fumarate;
3-(tert-butoxy)-3-oxopropyl cyclooctyl fumarate;
3-(tert-butoxy)-3-oxopropyl cyclohexyl fumarate;
(S)-1-(tert-butoxy)-1-oxopropan-2-yl cyclooctyl fumarate;
Cyclooctyl (3-((4-methoxybenzyl)oxy)-2,2-dimethyl-3-oxopropyl) fumarate;
2-(tert-butoxy)-2-oxoethyl spiro[3.3]heptan-2-yl fumarate;
2-(tert-butoxy)-2-oxoethyl cycloheptyl fumarate;
Cyclooctyl (4-((4-methoxybenzyl)oxy)-4-oxobutan-2-yl) fumarate;
(R)-1-(tert-butoxy)-1-oxopropan-2-yl cyclooctyl fumarate;
(R)-2-(tert-butoxy)-2-oxoethyl octan-2-yl fumarate;
(R)-3-(tert-butoxy)-3-oxopropyl octan-2-yl fumarate.

102. (canceled)

103: A process for the preparation of compounds of formula (I) or a salt thereof, which comprises reacting a compound of formula (II): 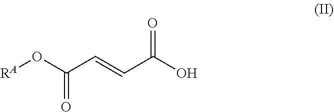
or a salt thereof, wherein R^{A} is 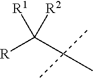
wherein R is C₄₋₁₀ alkyl, and R¹ and R² are independently selected from the group consisting of H, C₁₋₄ alkyl and C₁₋₄ haloalkyl or R¹ and R² join to form a C₃₋₄ cycloalkyl ring; wherein R is optionally substituted by one or more R^{a} wherein R^{a} is independently selected from the group consisting of halo, C₁₋₂ haloalkyl and C₁₋₂ haloalkoxy; or
R is selected from the group consisting of C₆₋₁₀ cycloalkyl, phenyl and 5- or 6-membered heteroaryl, and R¹ and R² are independently selected from the group consisting of H, C₁₋₄ alkyl and C₁₋₄ haloalkyl, or R¹ and R² join to form a C₃₋₄ cycloalkyl ring or a 4-6-membered heterocyclic ring, wherein the C₃₋₄ cycloalkyl ring is optionally substituted by methyl, halo or cyano; wherein R is optionally substituted by one or more R^{b} wherein R^{b} is independently selected from the group consisting of halo, C₁₋₄ alkyl, C₁₋₄ haloalkyl, C₁₋₄ alkoxy, C₁₋₄ haloalkoxy and cyano; or
R is H, methyl or CF₃ and R¹ and R² are joined to form a C₄₋₁₀ cycloalkyl ring, wherein the C₄₋₁₀ cycloalkyl ring is optionally substituted by one or more R^{c} wherein R^{c} is independently selected from the group consisting of halo, C₁₋₂ alkyl, C₁₋₂ haloalkyl, C₁₋₂ alkoxy and C₁₋₂ haloalkoxy, and/or wherein the C₄₋₁₀ cycloalkyl ring is optionally substituted by two R^{c} groups wherein the two R^{C} groups are attached to the same carbon atom and are joined to form a C₄₋₆ cycloalkyl ring;
with:
(i) X—R^{B} or a salt, thereof, wherein X is halo and R^{B} is defined in claim 1; or
(ii) X—R^{B}—P or a salt thereof, followed by removing protecting group P, wherein R^{B} is selected from the group consisting of CH₂COOH, CH₂CH₂COOH, CH₂tetrazolyl and CH₂CH₂tetrazolyl, wherein R^{B} is optionally substituted on an available carbon atom by one or more R^{B′} wherein R^{B′} is selected from the group consisting of difluoromethyl, trifluoromethyl and methyl, and/or wherein R^{B} is optionally substituted by two R^{B′} groups, attached to the same carbon atom, that are joined to form a C₃₋₆ cycloalkyl or a 4-6-membered heterocyclic ring, P is a carboxylic acid protecting group and X is halo.

104-110. (canceled)

111: A method of treating or preventing an inflammatory disease or a disease associated with an undesirable immune response, which comprises administering a compound according to claim 98.

112-115. (canceled)

116: The method according to claim 111, wherein the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease, autoimmune paraneoplastic retinopathy, transplantation rejection (including antibody-mediated and T cell-mediated forms), multiple sclerosis, transverse myelitis, ischaemia-reperfusion injury, AGE-induced genome damage, an inflammatory bowel disease, primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behçet's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, Behcet's disease, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), amilial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutières syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders, and renal inflammatory disorders.

117-127. (canceled)
